SEQUENCE LISTING (20 sequences)

SEQ ID NO: 1 - protein, 680 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 2 - protein, 708 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKEDENSWFNLFVIHQNRSKHGSTNFIPEQFLDDFIDLVIWGHEHECKIAPTKNEQQLFYISQPGSSVVTSLSPGEAVKKHVGLLRIKGRKMNMHKIPLHTVRQFFMEDIVLANHPDIFNPDNPKVTQAIQSFCLEKIEEMLENAERERLGNSHQPEKPLVRLRVDYSGGFEPFSVLRFSQKFVDRVANPKDIIHFFRHREQKEKTGEEINFGKLITKPSEGTTLRVEDLVKQYFQTAEKNVQLSLLTERGMGEAVQEFVDKEEKDAIEELVKYQLEKTQRFLKERHIDALEDKIDEEVRRFRETRQKNTNEEDDEVREAMTRARALRSQSEESASAFSADDLMSIDLAEQMANDSDDSISAATNKGRGRGRGRRGGRGQNSASRGGSQRGRADTGLETSTRSRNSKTAVSASRNMSIIDAFKSTRQQPSRNVTTKNYSEVIEVDESDVEEDIFPTTSKTDQRWSSTSSSKIMSQSQVSKGVDFESSEDDDDDPFMNTSSLRRNRR

SEQ ID NO: 3 - protein, 206 aa, Homo sapiens
MSTADALDDENTFKILVATDIHLGFMEKDAVRGNDTFVTLDEILRLAQENEVDFILLGGDLFHENKPSRKTLHTCLELLRKYCMGDRPVQFEILSDQSVNFGFSKFPWVNYQDGNLNISIPVFSIHGNHDDPTGADALCALDILSCAGFVNHFGRSMSVEKIDISPVLLQKGSTKIALYGLGSIPDERLYRMFVNKKVTMLRPKED

SEQ ID NO: 4 - protein, 680 aa, Homo sapiens [sequence omitted]

SEQ ID NO: 5 - protein, 1312 aa, Homo sapiens
MSRIEKMSILGVRSFGIEDKDKQIITFFSPLTILVGPNGAGKTTIIECLKYICTGDFPPGTKGNTFVHDPKVAQETDVRAQIRLQFRDVNGELIAVQRSMVCTQKSKKTEFKTLEGVITRTKHGEKVSLSSKCAEIDREMISSLGVSKAVLNNVIFCHQEDSNWPLSEGKALKQKFDEIFSATRYIKALETLRQVRQTQGQKVKEYQMELKYLKQYKEKACEIRDQITSKEAQLTSSKEIVKSYENELDPLKNRLKEIEHNLSKIMKLDNEIKALDSRKKQMEKDNSELEEKMEKVFQGTDEQLNDLYHNHQRTVREKERKLVDCHRELEKLNKESRLLNQEKSELLVEQGRLQLQADRHQEHIRARDSLIQSLATQLELDGFERGPFSERQIKNFHKLVRERQEGEAKTANQLMNDFAEKETLKQKQIDEIRDKKTGLGRIIELKSEILSKKQNELKNVKYELQQLEGSSDRILELDQELIKAERELSKAEKNSNVETLKMEVISLQNEKADLDRTLRKLDQEMEQLNHHTTTRTQMEMLTKDKADKDEQIRKIKSRHSDELTSLLGYFPNKKQLEDWLHSKSKEINQTRDRLAKLNKELASSEQNKNHINNELKRKEEQLSSYEDKLFDVCGSQDFESDLDRLKEEIEKSSKQRAMLAGATAVYSQFITQLTDENQSCCPVCQRVFQTEAELQEVISDLQSKLRLAPDKLKSTESELKKKEKRRDEMLGLVPMRQSIIDLKEKEIPELRNKLQNVNRDIQRLKNDIEEQETLLGTIMPEEESAKVCLTDVTIMERFQMELKDVERKIAQQAAKLQGIDLDRTVQQVNQEKQEKQHKLDTVSSKIELNRKLIQDQQEQIQHLKSTTNELKSEKLQISTNLQRRQQLEEQTVELSTEVQSLYREIKDAKEQVSPLETTLEKFQQEKEELINKKNTSNKIAQDKLNDIKEKVKNIHGYMKDIENYIQDGKDDYKKQKETELNKVIAQLSECEKHKEKINEDMRLMRQDIDTQKIQERWLQDNLTLRKRNEELKEVEEERKQHLKEMGQMQVLQMKSEHQKLEENIDNIKRNHNLALGRQKGYEEEIIHFKKELREPQFRDAEEKYREMMIVMRTTELVNKDLDIYYKTLDQAIMKFHSMKMEEINKIIRDLWRSTYRGQDIEYIEIRSDADENVSASDKRRNYNYRVVMLKGDTALDMRGRCSAGQKVLASLIIRLALAETFCLNCGIIALDEPTTNLDRENIESLAHALVEIIKSRSQQRNFQLLVITHDEDFVELLGRSEYVEKFYRIKKNIDQCSEIVKCSVSSLGFNVH

SEQ ID NO: 6 - protein, 1312 aa, Homo sapiens; identical to SEQ ID NO: 5

SEQ ID NO: 7 - protein, 557 aa, Homo sapiens
MSRIEKMSILGVRSFGIEDKDKQIITFFSPLTILVGPNGAGKTTIIECLKYICTGDFPPGTKGNTFVHDPKVAQETDVRAQIRLQFRDVNGELIAVQRSMVCTQKSKKTEFKTLEGVITRTKHGEKVSLSSKCAEIDREMISSLGVSKAVLNNVIFCHQEDSNWPLSEGKALKQKFDEIFSATRYIKALETLRQVRQTQGQKVKEYQMELKYLKQYKEKACEIRDQITSKEAQLTSSKEIVKSYENELDPLKNRLKEIEHNLSKIMKLDNEIKALDSRKKQMEKDNSELEEKMEKVFQGTDEQLNDLYHNHQRTVREKERKLVDCHRELEKLNKESRLLNQEKSELLVEQGRLQLQADRHQEHIRARDSLIQSLATQLELDGFERGPFSERQIKNFHKLVRERQEGEAKTANQLMNDFAEKETLKQKQIDEIRDKKTGLGRIIELKSEILSKKQNELKNVKYELQQLEGSSDRILELDQELIKAERELSKAEKNSNVETLKMEVISLQNEKADLDRTLRKLDQEMEQLNHHTTTRTQMEMLTKDKADKDEQIRKKKK

SEQ ID NO: 8 - protein, 754 aa, Homo sapiens
MWKLLPAAGPAGGEPYRLLTGVEYVVGRKNCAILIENDQSISRNHAVLTANFSVTNLSQTDEIPVLTLKDNSKYGTFVNEEKMQNGFSRTLKSGDGITFGVFGSKFRIEYEPLVACSSCLDVSGKTALNQAILQLGGFTVNNWTEECTHLVMVSVKVTIKTICALICGRPIVKPEYFTEFLKAVESKKQPPQIESFYPPLDEPSIGSKNVDLSGRQERKQIFKGKTFIFLNAKQHKKLSSAVVFGGGEARLITEENEEEHNFFLAPGTCVVDTGITNSQTLIPDCQKKWIQSIMDMLQRQGLRPIPEAEIGLAVIFMTTKNYCDPQGHPSTGLKTTTPGPSLSQGVSVDEKLMPSAPVNTTTYVADTESEQADTWDLSERPKEIKVSKMEQKFRMLSQDAPTVKESCKTSSNNNSMVSNTLAKMRIPNYQLSPTKLPSINKSKDRASQQQQTNSIRNYFQPSTKKRERDEENQEMSSCKSARIETSCSLLEQTQPATPSLWKNKEQHLSENEPVDTNSDNNLFTDTDLKSIVKNSASKSHAAEKLRSNKKREMDDVAIEDEVLEQLFKDTKPELEIDVKVQKQEEDVNVRKRPRMDIETNDTFSDEAVPESSKISQENEIGKKRELKEDSLWSAKEISNNDKLQDDSEMLPKKLLLTEFRSLVIKNSTSRNPSGINDDYGQLKNFKKFKKVTYPGAGKLPHIIGGSDLIAHHARKNTELEEWLRQEMEVQNQHAKEESLADDLFRYNPYLKRRR

SEQ ID NO: 9 - protein, 754 aa, Homo sapiens; identical to SEQ ID NO: 8

SEQ ID NO: 10 - protein, 754 aa, Homo sapiens; identical to SEQ ID NO: 8

SEQ ID NO: 11 - DNA, 2040 bp, Artificial Sequence
  Predictive cDNA (degenerate) based on NP_005581 amino acid sequence
atgwsnacngcngaygcnytngaygaygaraayacnttyaarathytngtngcnacngayathcayytnggnttyatggaraargaygcngtnmgnggnaaygayacnttygtnacnytngaygarathytnmgnytngcncargaraaygargtngayttyathytnytnggnggngayytnttycaygaraayaarccnwsnmgnaaracnytncayacntgyytngarytnytnmgnaartaytgyatgggngaymgnccngtncarttygarathytnwsngaycarwsngtnaayttyggnttywsnaarttyccntgggtnaaytaycargayggnaayytnaayathwsnathccngtnttywsnathcayggnaaycaygaygayccnacnggngcngaygcnytntgygcnytngayathytnwsntgygcnggnttygtnaaycayttyggnmgnwsnatgwsngtngaraarathgayathwsnccngtnytnytncaraarggnwsnacnaarathgcnytntayggnytnggnwsnathccngaygarmgnytntaymgnatgttygtnaayaaraargtnacnatgytnmgnccnaargargaygaraaywsntggttyaayytnttygtnathcaycaraaymgnwsnaarcayggnwsnacnaayttyathccngarcarttyytngaygayttyathgayytngtnathtggggncaygarcaygartgyaarathgcnccnacnaaraaygarcarcarytnttytayathwsncarccnggnwsnwsngtngtnacnwsnytnwsnccnggngargcngtnaaraarcaygtnggnytnytnmgnathaarggnmgnaaratgaayatgcayaarathccnytncayacngtnmgncarttyttyatggargayathgtnytngcnaaycayccngayathttyaayccngayaayccnaargtnacncargcnathcarwsnttytgyytngaraarathgargaratgytngaraaygcngarmgngarmgnytnggnaaywsncaycarccngaraarccnytngtnmgnytnmgngtngaytaywsnggnggnttygarccnttywsngtnytnmgnttywsncaraarttygtngaymgngtngcnaayccnaargayathathcayttyttymgncaymgngarcaraargaraaracnggngargarathaayttyggnaarytnathacnaarccnwsngarggnacnacnytnmgngtngargayytngtnaarcartayttycaracngcngaraaraaygtncarytnwsnytnytnacngarmgnggnatgggngargcngtncargarttygtngayaargargaraargaygcnathgargarytngtnaartaycarytngaraaracncarmgnttyytnaargarmgncayathgaygcnytngargayaarathgaygargargtnmgnmgnttymgngaracnmgncaraaraayacnaaygargargaygaygargtnmgngargcnatgacnmgngcnmgngcnytnmgnwsncarwsngargarwsngcnwsngcnttywsngcngaygayytnatgwsnathgayytngcngarcaratggcnaaygaywsngaygaywsnathwsngcngcnacnaayaarggnmgnggnmgnggnmgnggnmgnmgnggnggnmgnggncaraaywsngcnwsnmgnggnggnwsncarmgnggnmgngcnttyaarwsnacnmgncarcarccnwsnmgnaaygtnacnacnaaraaytaywsngargtnathgargtngaygarwsngaygtngargargayathttyccnacnacnwsnaaracngaycarmgntggwsnwsnacnwsnwsnwsnaarathatgwsncarwsncargtnwsnaarggngtngayttygarwsnwsngargaygaygaygaygayccnttyatgaayacnwsnwsnytnmgnmgnaaymgnmgn

SEQ ID NO: 12 - DNA, 2124 bp, Artificial Sequence
  Predictive cDNA (degenerate) based on NP_005582 amino acid sequence
atgwsnacngcngaygcnytngaygaygaraayacnttyaarathytngtngcnacngayathcayytnggnttyatggaraargaygcngtnmgnggnaaygayacnttygtnacnytngaygarathytnmgnytngcncargaraaygargtngayttyathytnytnggnggngayytnttycaygaraayaarccnwsnmgnaaracnytncayacntgyytngarytnytnmgnaartaytgyatgggngaymgnccngtncarttygarathytnwsngaycarwsngtnaayttyggnttywsnaarttyccntgggtnaaytaycargayggnaayytnaayathwsnathccngtnttywsnathcayggnaaycaygaygayccnacnggngcngaygcnytntgygcnytngayathytnwsntgygcnggnttygtnaaycayttyggnmgnwsnatgwsngtngaraarathgayathwsnccngtnytnytncaraarggnwsnacnaarathgcnytntayggnytnggnwsnathccngaygarmgnytntaymgnatgttygtnaayaaraargtnacnatgytnmgnccnaargargaygaraaywsntggttyaayytnttygtnathcaycaraaymgnwsnaarcayggnwsnacnaayttyathccngarcarttyytngaygayttyathgayytngtnathtggggncaygarcaygartgyaarathgcnccnacnaaraaygarcarcarytnttytayathwsncarccnggnwsnwsngtngtnacnwsnytnwsnccnggngargcngtnaaraarcaygtnggnytnytnmgnathaarggnmgnaaratgaayatgcayaarathccnytncayacngtnmgncarttyttyatggargayathgtnytngcnaaycayccngayathttyaayccngayaayccnaargtnacncargcnathcarwsnttytgyytngaraarathgargaratgytngaraaygcngarmgngarmgnytnggnaaywsncaycarccngaraarccnytngtnmgnytnmgngtngaytaywsnggnggnttygarccnttywsngtnytnmgnttywsncaraarttygtngaymgngtngcnaayccnaargayathathcayttyttymgncaymgngarcaraargaraaracnggngargarathaayttyggnaarytnathacnaarccnwsngarggnacnacnytnmgngtngargayytngtnaarcartayttycaracngcngaraaraaygtncarytnwsnytnytnacngarmgnggnatgggngargcngtncargarttygtngayaargargaraargaygcnathgargarytngtnaartaycarytngaraaracncarmgnttyytnaargarmgncayathgaygcnytngargayaarathgaygargargtnmgnmgnttymgngaracnmgncaraaraayacnaaygargargaygaygargtnmgngargcnatgacnmgngcnmgngcnytnmgnwsncarwsngargarwsngcnwsngcnttywsngcngaygayytnatgwsnathgayytngcngarcaratggcnaaygaywsngaygaywsnathwsngcngcnacnaayaarggnmgnggnmgnggnmgnggnmgnmgnggnggnmgnggncaraaywsngcnwsnmgnggnggnwsncarmgnggnmgngcngayacnggnytngaracnwsnacnmgnwsnmgnaaywsnaaracngcngtnwsngcnwsnmgnaayatgwsnathathgaygcnttyaarwsnacnmgncarcarccnwsnmgnaaygtnacnacnaaraaytaywsngargtnathgargtngaygarwsngaygtngargargayathttyccnacnacnwsnaaracngaycarmgntggwsnwsnacnwsnwsnwsnaarathatgwsncarwsncargtnwsnaarggngtngayttygarwsnwsngargaygaygaygaygayccnttyatgaayacnwsnwsnytnmgnmgnaaymgnmgn

SEQ ID NO: 13 - DNA, 618 bp, Artificial Sequence
  Predictive cDNA (degenerate) based on AAH05241 amino acid sequence
atgwsnacngcngaygcnytngaygaygaraayacnttyaarathytngtngcnacngayathcayytnggnttyatggaraargaygcngtnmgnggnaaygayacnttygtnacnytngaygarathytnmgnytngcncargaraaygargtngayttyathytnytnggnggngayytnttycaygaraayaarccnwsnmgnaaracnytncayacntgyytngarytnytnmgnaartaytgyatgggngaymgnccngtncarttygarathytnwsngaycarwsngtnaayttyggnttywsnaarttyccntgggtnaaytaycargayggnaayytnaayathwsnathccngtnttywsnathcayggnaaycaygaygayccnacnggngcngaygcnytntgygcnytngayathytnwsntgygcnggnttygtnaaycayttyggnmgnwsnatgwsngtngaraarathgayathwsnccngtnytnytncaraarggnwsnacnaarathgcnytntayggnytnggnwsnathccngaygarmgnytntaymgnatgttygtnaayaaraargtnacnatgytnmgnccnaargargay

SEQ ID NO: 14 - DNA, 2040 bp, Artificial Sequence
  Predictive cDNA (degenerate) based on AAC78721 amino acid sequence
atgwsnacngcngaygcnytngaygaygaraayacnttyaarathytngtngcnacngayathcayytnggnttyatggaraargaygcngcnmgnggnaaygayacnttygtnacnytngaygarathytnmgnytngcncargaraaygargtngayttyathytnytnggnggngayytnttycaygaraayaarccnwsnmgnaaracnytncayacntgyytngarytnytnmgnaartaytgyatgggngaymgnccngtncarttygarathytnwsngaycarwsngtnaayttyggnttywsnaarttyccntgggtnaaytaycargayggnaayytnaayathwsnathccngtnttywsnathcayggnaaycaygaygayccnacnggngcngaygcnytntgygcnytngayathytnwsntgygcnggnttygtnaaycayttyggnmgnwsnatgwsngtngaraarathgayathwsnccngtnytnytncaraarggnwsnacnaarathgcnytntayggnytnggnwsnathccngaygarmgnytntaymgnatgttygtnaayaaraargtnacnatgytnmgnccnaargargaygaraaywsntggttyaayytnttygtnathcaycaraaymgnwsnaarcayggnwsnacnaayttyathccngarcarttyytngaygayttyathgayytngtnathtggggncaygarcaygartgyaarathgcnccnacnaaraaygarcarcarytnttytayathwsncarccnggnwsnwsngtngtnacnwsnytnwsnccnggngargcngtnaaraarcaygtnggnytnytnmgnathaarggnmgnaaratgaayatgcayaarathccnytncayacngtnmgncarttyttyatggargayathgtnytngcnaaycayccngayathttyaayccngayaayccnaargtnacncargcnathcarwsnttytgyytngaraarathgargaratgytngaraaygcngarmgngarmgnytnggnaaywsncaycarccngaraarccnytngtnmgnytnmgngtngaytaywsnggnggnttygarccnttywsngtnytnmgnttywsncaraarttygtngaymgngtngcnaayccnaargayathathcayttyttymgncaymgngarcaraargaraaracnggngargarathaayttyggnaarytnathacnaarccnwsngarggnacnacnytnmgngtngargayytngtnaarcartayttycaracngcngaraaraaygtncarytnwsnytnytnacngarmgnggnatgggngargcngtncargarttygtngayaargargaraargaygcnathgargarytngtnaartaycarytngaraaracncarmgnttyytnaargarmgncayathgaygcnytngargayaarathgaygargargtnmgnmgnttymgngaracnmgncaraaraayacnaaygargargaygaygargtnmgngargcnatgacnmgngcnmgngcnytnmgnwsncarwsngargarwsngcnwsngcnttywsngcngaygayytnatgwsnathgayytngcngarcaratggcnaaygaywsngaygaywsnathwsngcngcnacnaayaarggnmgnggnmgnggnmgnggnmgnmgnggnggnmgnggncaraaywsngcnwsnmgnggnggnwsncarmgnggnmgngcnttyaarwsnacnmgncarcarccnwsnmgnaaygtnacnacnaaraaytaywsngargtnathgargtngaygarwsngaygtngargargayathttyccnacnacnwsnaaracngaycarmgntggwsnwsnacnwsnwsnwsnaarathatgwsncarwsncargtnwsnaarggngtngayttygarwsnwsngargaygaygaygaygayccnttyatgaayacnwsnwsnytnmgnmgnaaymgnmgn

SEQ ID NO: 15 - DNA, 3936 bp, Artificial Sequence
  Predictive cDNA (degenerate) based on AAB07119 amino acid sequence
atgwsnmgnathgaraaratgwsnathytnggngtnmgnwsnttyggnathgargayaargayaarcarathathacnttyttywsnccnytnacnathytngtnggnccnaayggngcnggnaaracnacnathathgartgyytnaartayathtgyacnggngayttyccnccnggnacnaarggnaayacnttygtncaygayccnaargtngcncargaracngaygtnmgngcncarathmgnytncarttymgngaygtnaayggngarytnathgcngtncarmgnwsnatggtntgyacncaraarwsnaaraaracngarttyaaracnytngarggngtnathacnmgnacnaarcayggngaraargtnwsnytnwsnwsnaartgygcngarathgaymgngaratgathwsnwsnytnggngtnwsnaargcngtnytnaayaaygtnathttytgycaycargargaywsnaaytggccnytnwsngarggnaargcnytnaarcaraarttygaygarathttywsngcnacnmgntayathaargcnytngaracnytnmgncargtnmgncaracncarggncaraargtnaargartaycaratggarytnaartayytnaarcartayaargaraargcntgygarathmgngaycarathacnwsnaargargcncarytnacnwsnwsnaargarathgtnaarwsntaygaraaygarytngayccnytnaaraaymgnytnaargarathgarcayaayytnwsnaarathatgaarytngayaaygarathaargcnytngaywsnmgnaaraarcaratggaraargayaaywsngarytngargaraaratggaraargtnttycarggnacngaygarcarytnaaygayytntaycayaaycaycarmgnacngtnmgngaraargarmgnaarytngtngaytgycaymgngarytngaraarytnaayaargarwsnmgnytnytnaaycargaraarwsngarytnytngtngarcarggnmgnytncarytncargcngaymgncaycargarcayathmgngcnmgngaywsnytnathcarwsnytngcnacncarytngarytngayggnttygarmgnggnccnttywsngarmgncarathaaraayttycayaarytngtnmgngarmgncargarggngargcnaaracngcnaaycarytnatgaaygayttygcngaraargaracnytnaarcaraarcarathgaygarathmgngayaaraaracnggnytnggnmgnathathgarytnaarwsngarathytnwsnaaraarcaraaygarytnaaraaygtnaartaygarytncarcarytngarggnwsnwsngaymgnathytngarytngaycargarytnathaargcngarmgngarytnwsnaargcngaraaraaywsnaaygtngaracnytnaaratggargtnathwsnytncaraaygaraargcngayytngaymgnacnytnmgnaarytngaycargaratggarcarytnaaycaycayacnacnacnmgnacncaratggaratgytnacnaargayaargcngayaargaygarcarathmgnaarathaarwsnmgncaywsngaygarytnacnwsnytnytnggntayttyccnaayaaraarcarytngargaytggytncaywsnaarwsnaargarathaaycaracnmgngaymgnytngcnaarytnaayaargarytngcnwsnwsngarcaraayaaraaycayathaayaaygarytnaarmgnaargargarcarytnwsnwsntaygargayaarytnttygaygtntgyggnwsncargayttygarwsngayytngaymgnytnaargargarathgaraarwsnwsnaarcarmgngcnatgytngcnggngcnacngcngtntaywsncarttyathacncarytnacngaygaraaycarwsntgytgyccngtntgycarmgngtnttycaracngargcngarytncargargtnathwsngayytncarwsnaarytnmgnytngcnccngayaarytnaarwsnacngarwsngarytnaaraaraargaraarmgnmgngaygaratgytnggnytngtnccnatgmgncarwsnathathgayytnaargaraargarathccngarytnmgnaayaarytncaraaygtnaaymgngayathcarmgnytnaaraaygayathgargarcargaracnytnytnggnacnathatgccngargargarwsngcnaargtntgyytnacngaygtnacnathatggarmgnttycaratggarytnaargaygtngarmgnaarathgcncarcargcngcnaarytncarggnathgayytngaymgnacngtncarcargtnaaycargaraarcargaraarcarcayaarytngayacngtnwsnwsnaarathgarytnaaymgnaarytnathcargaycarcargarcarathcarcayytnaarwsnacnacnaaygarytnaarwsngaraarytncarathwsnacnaayytncarmgnmgncarcarytngargarcaracngtngarytnwsnacngargtncarwsnytntaymgngarathaargaygcnaargarcargtnwsnccnytngaracnacnytngaraarttycarcargaraargargarytnathaayaaraaraayacnwsnaayaarathgcncargayaarytnaaygayathaargaraargtnaaraayathcayggntayatgaargayathgaraaytayathcargayggnaargaygaytayaaraarcaraargaracngarytnaayaargtnathgcncarytnwsngartgygaraarcayaargaraarathaaygargayatgmgnytnatgmgncargayathgayacncaraarathcargarmgntggytncargayaayytnacnytnmgnaarmgnaaygargarytnaargargtngargargarmgnaarcarcayytnaargaratgggncaratgcargtnytncaratgaarwsngarcaycaraarytngargaraayathgayaayathaarmgnaaycayaayytngcnytnggnmgncaraarggntaygargargarathathcayttyaaraargarytnmgngarccncarttymgngaygcngargaraartaymgngaratgatgathgtnatgmgnacnacngarytngtnaayaargayytngayathtaytayaaracnytngaycargcnathatgaarttycaywsnatgaaratggargarathaayaarathathmgngayytntggmgnwsnacntaymgnggncargayathgartayathgarathmgnwsngaygcngaygaraaygtnwsngcnwsngayaarmgnmgnaaytayaaytaymgngtngtnatgytnaarggngayacngcnytngayatgmgnggnmgntgywsngcnggncaraargtnytngcnwsnytnathathmgnytngcnytngcngaracnttytgyytnaaytgyggnathathgcnytngaygarccnacnacnaayytngaymgngaraayathgarwsnytngcncaygcnytngtngarathathaarwsnmgnwsncarcarmgnaayttycarytnytngtnathacncaygaygargayttygtngarytnytnggnmgnwsngartaygtngaraarttytaymgnathaaraaraayathgaycartgywsngarathgtnaartgywsngtnwsnwsnytnggnttyaaygtncay

SEQ ID NO: 16 - DNA, 3936 bp, Artificial Sequence; identical to SEQ ID NO: 15
  Predictive cDNA (degenerate) based on NP_005723 amino acid sequence

SEQ ID NO: 17 - DNA, 1671 bp, Artificial Sequence
  Predictive cDNA (degenerate) based on AAH62603 amino acid sequence
atgwsnmgnathgaraaratgwsnathytnggngtnmgnwsnttyggnathgargayaargayaarcarathathacnttyttywsnccnytnacnathytngtnggnccnaayggngcnggnaaracnacnathathgartgyytnaartayathtgyacnggngayttyccnccnggnacnaarggnaayacnttygtncaygayccnaargtngcncargaracngaygtnmgngcncarathmgnytncarttymgngaygtnaayggngarytnathgcngtncarmgnwsnatggtntgyacncaraarwsnaaraaracngarttyaaracnytngarggngtnathacnmgnacnaarcayggngaraargtnwsnytnwsnwsnaartgygcngarathgaymgngaratgathwsnwsnytnggngtnwsnaargcngtnytnaayaaygtnathttytgycaycargargaywsnaaytggccnytnwsngarggnaargcnytnaarcaraarttygaygarathttywsngcnacnmgntayathaargcnytngaracnytnmgncargtnmgncaracncarggncaraargtnaargartaycaratggarytnaartayytnaarcartayaargaraargcntgygarathmgngaycarathacnwsnaargargcncarytnacnwsnwsnaargarathgtnaarwsntaygaraaygarytngayccnytnaaraaymgnytnaargarathgarcayaayytnwsnaarathatgaarytngayaaygarathaargcnytngaywsnmgnaaraarcaratggaraargayaaywsngarytngargaraaratggaraargtnttycarggnacngaygarcarytnaaygayytntaycayaaycaycarmgnacngtnmgngaraargarmgnaarytngtngaytgycaymgngarytngaraarytnaayaargarwsnmgnytnytnaaycargaraarwsngarytnytngtngarcarggnmgnytncarytncargcngaymgncaycargarcayathmgngcnmgngaywsnytnathcarwsnytngcnacncarytngarytngayggnttygarmgnggnccnttywsngarmgncarathaaraayttycayaarytngtnmgngarmgncargarggngargcnaaracngcnaaycarytnatgaaygayttygcngaraargaracnytnaarcaraarcarathgaygarathmgngayaaraaracnggnytnggnmgnathathgarytnaarwsngarathytnwsnaaraarcaraaygarytnaaraaygtnaartaygarytncarcarytngarggnwsnwsngaymgnathytngarytngaycargarytnathaargcngarmgngarytnwsnaargcngaraaraaywsnaaygtngaracnytnaaratggargtnathwsnytncaraaygaraargcngayytngaymgnacnytnmgnaarytngaycargaratggarcarytnaaycaycayacnacnacnmgnacncaratggaratgytnacnaargayaargcngayaargaygarcarathmgnaaraaraaraar

SEQ ID NO: 18 - DNA, 2262 bp, Artificial Sequence
  Predictive cDNA (degenerate) based on BAA28616 amino acid sequence
atgtggaarytnytnccngcngcnggnccngcnggnggngarccntaymgnytnytnacnggngtngartaygtngtnggnmgnaaraaytgygcnathytnathgaraaygaycarwsnathwsnmgnaaycaygcngtnytnacngcnaayttywsngtnacnaayytnwsncaracngaygarathccngtnytnacnytnaargayaaywsnaartayggnacnttygtnaaygargaraaratgcaraayggnttywsnmgnacnytnaarwsnggngayggnathacnttyggngtnttyggnwsnaarttymgnathgartaygarccnytngtngcntgywsnwsntgyytngaygtnwsnggnaaracngcnytnaaycargcnathytncarytnggnggnttyacngtnaayaaytggacngargartgyacncayytngtnatggtnwsngtnaargtnacnathaaracnathtgygcnytnathtgyggnmgnccnathgtnaarccngartayttyacngarttyytnaargcngtngarwsnaaraarcarccnccncarathgarwsnttytayccnccnytngaygarccnwsnathggnwsnaaraaygtngayytnwsnggnmgncargarmgnaarcarathttyaarggnaaracnttyathttyytnaaygcnaarcarcayaaraarytnwsnwsngcngtngtnttyggnggnggngargcnmgnytnathacngargaraaygargargarcayaayttyttyytngcnccnggnacntgygtngtngayacnggnathacnaaywsncaracnytnathccngaytgycaraaraartggathcarwsnathatggayatgytncarmgncarggnytnmgnccnathccngargcngarathggnytngcngtnathttyatgacnacnaaraaytaytgygayccncarggncayccnwsnacnggnytnaaracnacnacnccnggnccnwsnytnwsncarggngtnwsngtngaygaraarytnatgccnwsngcnccngtnaayacnacnacntaygtngcngayacngarwsngarcargcngayacntgggayytnwsngarmgnccnaargarathaargtnwsnaaratggarcaraarttymgnatgytnwsncargaygcnccnacngtnaargarwsntgyaaracnwsnwsnaayaayaaywsnatggtnwsnaayacnytngcnaaratgmgnathccnaaytaycarytnwsnccnacnaarytnccnwsnathaayaarwsnaargaymgngcnwsncarcarcarcaracnaaywsnathmgnaaytayttycarccnwsnacnaaraarmgngarmgngaygargaraaycargaratgwsnwsntgyaarwsngcnmgnathgaracnwsntgywsnytnytngarcaracncarccngcnacnccnwsnytntggaaraayaargarcarcayytnwsngaraaygarccngtngayacnaaywsngayaayaayytnttyacngayacngayytnaarwsnathgtnaaraaywsngcnwsnaarwsncaygcngcngaraarytnmgnwsnaayaaraarmgngaratggaygaygtngcnathgargaygargtnytngarcarytnttyaargayacnaarccngarytngarathgaygtnaargtncaraarcargargargaygtnaaygtnmgnaarmgnccnmgnatggayathgaracnaaygayacnttywsngaygargcngtnccngarwsnwsnaarathwsncargaraaygarathggnaaraarmgngarytnaargargaywsnytntggwsngcnaargarathwsnaayaaygayaarytncargaygaywsngaratgytnccnaaraarytnytnytnacngarttymgnwsnytngtnathaaraaywsnacnwsnmgnaayccnwsnggnathaaygaygaytayggncarytnaaraayttyaaraarttyaaraargtnacntayccnggngcnggnaarytnccncayathathggnggnwsngayytnathgcncaycaygcnmgnaaraayacngarytngargartggytnmgncargaratggargtncaraaycarcaygcnaargargarwsnytngcngaygayytnttymgntayaayccntayytnaarmgnmgnmgn

SEQ ID NO: 19 - DNA, 2262 bp, Artificial Sequence; identical to SEQ ID NO: 18
  Predictive cDNA (degenerate) based on AAC62232 amino acid sequence

SEQ ID NO: 20 - DNA, 2262 bp, Artificial Sequence; identical to SEQ ID NO: 18
  Predictive cDNA (degenerate) based on AAS59158 amino acid sequence

(19) United States
(12) Patent Application Publication
(10) Pub. No.: US 2015/0313922 A1
(43) Pub. Date: Nov. 5, 2015

(54) COMPOSITIONS AND METHODS FOR THE TREATMENT OF CANCERS ASSOCIATED WITH A DEFICIENCY IN THE MRE11/RAD50/NBS1 DNA DAMAGE REPAIR COMPLEX

(71) Applicant: SLOAN-KETTERING INSTITUTE FOR CANCER RESEARCH, New York, NY (US)

(72) Inventors: John H.J. Petrini, New York, NY (US); Gaorav P. Gupta, New York, NY (US)

(21) Appl. No.: 14/647,806

(22) PCT Filed: Nov. 27, 2013
(86) PCT No.: PCT/US2013/072394

§ 371 Date: May 27, 2015

Related U.S. Application Data
(60) Provisional application No. 61/730,855, filed on Nov. 28, 2012.

(51) Int. Cl. A61K 31/675; A61K 31/196; A61K 33/04; A61K 31/198; A61K 31/4745; A61K 31/282; A61K 33/24; A61K 31/407; A61K 31/513; A61K 31/704; A61K 45/06; G01N 33/574; G01N 33/50; A61N 5/10; A61K 31/131

(52) U.S. Cl. CPC A61K 31/675; A61K 31/131; A61K 31/196; A61K 33/04; A61K 31/198; A61K 31/4745; A61K 31/282; A61K 33/24; A61K 31/407; A61K 31/513; A61K 31/704; A61K 45/06; G01N 33/57496; G01N 33/5011; A61N 5/10; G01N 2800/52; G01N 2333/47

(57) ABSTRACT

Provided are compositions and methods for the identification and treatment of cancers exhibiting reduced MRE11/RAD50/NBS1 (MRN) complex formation and/or functionality as well as methods for the identification and use of cytotoxic agents, including clastogenic agents, for the treatment of cancers exhibiting reduced MRN complex formation and/or functionality. Also provided are methods for detecting and treating cancers, in particular breast cancers, such as hormone-negative breast cancers (HNBCs) and triple-negative breast cancers (TNBCs), colorectal cancers, urothelial cancers, and other cancers that exhibit reduced MRN complex formation and/or functionality and are correspondingly sensitive to growth and/or survival inhibition by one or more cytotoxic agents.

### CROSS-REFERENCE TO RELATED APPLICATIONS

This application claims the benefit of U.S. Provisional Patent Application No. 61/730,855, filed Nov. 28, 2012, which provisional patent application is incorporated by reference in its entirety.

### GOVERNMENT SPONSORED RESEARCH OR DEVELOPMENT

This invention was made with government support under grants BC093518 and GM59413 awarded by the National Institutes of Health. The government has certain rights in the invention.

### SEQUENCE LISTING

The present application includes a Sequence Listing in electronic format as a txt file in ASCII format titled “60009_{—}0006WOU_SEQ_LIST_ST25.txt,” which was created on Nov. 26, 2013 and which has a size of 99,862 bytes. The contents of txt file “60009_{—}0006WOU_SEQ_LIST_ST25.txt” are incorporated by reference herein.

### BACKGROUND OF THE DISCLOSURE

1. Technical Field

The present disclosure relates, generally, to the treatment of cancers. More specifically, this disclosure concerns the identification and treatment of cancers exhibiting reduced MRE11/RAD50/NBS1 (MRN) complex formation and/or functionality and the identification and use of cytotoxic agents, including clastogenic and other chemotherapeutic compounds, and ionizing radiation sources for the treatment of cancers exhibiting reduced MRN complex formation and/or functionality. Provided are methods for detecting and treating cancers, in particular hormone negative breast cancers (HNBCs), triple negative breast cancers (TNBCs), colorectal cancers, urothelial cancers; and other cancers that exhibit reduced MRN complex formation and/or functionality and are correspondingly sensitive to growth and/or survival inhibition by one or more cytotoxic agents.

2. Description of the Related Art

Hormone negative breast cancers (HNBCs) exhibit reduced or undetectable levels of expression of receptors for the endocrine hormones estrogen (ER) and/or progesterone (PR). Triple negative breast cancers (TNBCs) are a subset of such hormone negative (i.e., ER⁻/PR⁻) breast cancers that also do not express a Her2/Neu translocation. That is, TNBCs are HER2⁻ HNBCs.

HNBCs, in particular TNBCs, are particularly difficult to treat because the reduction in hormone receptor levels reduces the range of suitable treatment options available to such patients. Moreover, HNBCs, including TMBCs, are typically more invasive than hormone positive breast cancers and often metastasize to tissues beyond the breast. HNBCs and TNBCs are also associated with reduced recurrence rates and decreased survival prognosis. Patients diagnosed with HNBCs often have limited time and/or endurance to be subjected to multiple treatment regimens before succumbing to the disease. Unfortunately, little is known about how HNBCs and TNBCs respond to existing cancer therapies.

The maintenance of genome stability depends on the DNA damage response (DDR) network—a functional network comprising signal transduction, cell cycle checkpoint regulation, and double-strand DNA break repair. Theunissen et al., Mol. Cell 12:1511 (2003) and Stewart et al., Cell 99:577 (1999). The metabolism of DNA double-strand breaks governed by the DDR is important for preventing genomic alterations and sporadic cancers, and hereditary defects in this response cause debilitating human pathologies, including developmental defects and cancers.

The MRN complex, which is composed of the meiotic recombination 11 (MRE11), RAD50 and Nijmegen breakage syndrome 1 (NBS1; a/k/a Nibrin and p95) proteins is an apical sensor of DNA double strand breaks and mediates DNA repair (predominantly via homologous recombination (HR)), checkpoint activation, and initiation of an ATM-dependent signaling program. Functional impairment in the MRN complex, therefore, leads to an inability to tolerate multiple types of DNA damage. Recent insights into the structure and function of the MRN complex have been gained from in vitro structural analysis and studies of animal models in which the DDR response is deficient. Reviewed in Stracker and Petrini, Nature Rev. Mol. Cell Biol. 12:90-103 (2011).

### SUMMARY OF THE DISCLOSURE

The present disclosure is based upon the discovery that certain non-germline changes and mutations in Mre11, Rad50, and Nbs1 genes and/or in regions that regulate the expression of Mre11, Rad50, and/or Nbs1 genes, which result in a reduced cellular level of one or more MRE11, RAD50, and/or NBS1 protein and/or a reduced functionality of an MRE11, RAD50, and/or NBS1 protein, result in reduced MRE11/RAD50/NBS1 (MRN) complex formation and/or functionality in a cell. As a consequence of reduced MRN complex formation and/or functionality, such cells exhibit an enhanced sensitivity, as compared to cells without such non-germline changes or mutations, to growth and/or survival inhibition by certain cytotoxic agents, including certain clastogenic and other chemotherapeutic compounds, and sources of ionizing radiation.

It was further discovered that certain cancers, including breast cancers, such as hormone-negative breast cancers (HNBCs) and triple-negative breast cancers (TNBCs); colorectal cancers; urothelial cancers; and other cancers having reduced MRN complex formation and/or functionality also have a reduced capacity to repair double-strand breaks. Such cancers exhibit an increased sensitivity to certain cytotoxic agents, including certain clastogenic and other chemotherapeutic compounds, and sources of ionizing radiation and, because of this increased sensitivity, are susceptible to treatment with those cytotoxic agents. In particular, it was discovered that cancers exhibiting reduced MRN complex formation and/or functionality are susceptible to growth and/or survival inhibition by agents that induce double-strand DNA breaks, including certain clastogenic agents, such as clastogenic compounds and sources of ionizing radiation.

It will be understood that the enhanced sensitivity of cells, in particular cancer cells, to growth and/or survival inhibition by certain cytotoxic agents that results from reduced MRN complex formation and/or functionality, provides certain advantages to clinicians by affording them an opportunity to decide on and make recommendations for therapeutic regimens for the treatment of patients who are afflicted with cancers that are more responsive to certain cytotoxic agents, including certain clastogenic and other chemotherapeutic agents, than would have been expected had it not been discovered that those cancers possess an enhanced sensitivity to such agents.

In certain cases, therefore, it is predicted that less aggressive therapeutic regimens (e.g., lower dosages of otherwise toxic therapeutic agents) can be employed against cancers exhibiting reduced MRN complex formation and/or functionality without compromising therapeutic efficacy. As a result, it is contemplated that such less aggressive, yet efficacious, therapeutic regimens will minimize potential safety concerns and will exhibit reduced toxicity and increased tolerability, which, as a consequence, will permit a higher degree of patient compliance throughout the course of a given treatment program.

Within one embodiment, the present disclosure provides methods for identifying a cancer cell exhibiting reduced MRN complex formation and/or functionality, which cancer cell is, as a consequence of reduced MRN complex formation and/or functionality, sensitive to growth and/or survival inhibition by one or more cytotoxic agents, including one or more clastogenic compounds and/or one or more non-clastogenic chemotherapeutic compounds, and/or one or more sources of ionizing radiation.

Within another embodiment, the present disclosure provides methods for identifying a patient having a cancer cell exhibiting reduced MRN complex formation and/or functionality, which cancer cell is, as a consequence of reduced MRN complex formation and/or functionality, sensitive to growth and/or survival inhibition by the administration of one or more cytotoxic agents, including one or more clastogenic compounds and/or one or more non-clastogenic chemotherapeutic compounds, and/or one or more sources of ionizing radiation.

Within certain aspects of these methods, reduced MRN complex formation and/or functionality can be detected in a cancer cell by comparison of MRN complex formation and/or functionality in a cancer cell to MRN complex formation and/or functionality in a non-cancer cell having normal MRN complex formation and functionality. As described in further detail herein, MRN complex formation and/or functionality can, for example, be assessed through an analysis of nuclear foci, which result from the formation of MRN complexes at the location of chromosomal double-strand breaks within the nucleus of a cell.

Within other aspects of these methods, reduced MRN complex formation and/or functionality results from and can be determined by detecting reduced Mre11, Rad50, and/or Nbs1 gene expression and/or reduced MRE11, RAD50, and/or NBS1 protein level in a cancer cell as compared to Mre11, Rad50, and/or Nbs1 gene expression and/or MRE11, RAD50, and/or NBS1 protein level in a non-cancer cell that exhibits normal Mre11, Rad50, and/or Nbs1 gene expression and/or normal MRE11, RAD50, and/or NBS1 protein level and, therefore normal MRN complex formation and functionality.

Within yet other aspects of these methods, reduced MRN complex formation and/or functionality results from and can be determined by detecting one or more mutations, insertions, and/or deletions in an Mre11, Rad50, and/or Nbs1 gene, which mutations, insertions, and/or deletions reduce or eliminate one or more function of an MRE11, RAD50, and/or NBS1 protein in a cancer cell as compared to normal MRE11, RAD50, and/or NBS1 function in a non-cancer cell.

Within further aspects of these methods, the cancer is selected from the group consisting of a breast cancer, including a hormone-negative breast cancer (HNBC) and a triple-negative breast cancer (TNBC), a colorectal cancer, an urothelial cancers, and another cancer (i.e., a non-breast/colorectal/urothelial cancer) that exhibits reduced MRN complex formation and/or functionality.

Within a further embodiment, the present disclosure provides methods for inhibiting the growth and/or survival of a cancer cell that exhibits reduced MRN complex formation and/or functionality, by contacting the cancer cell with one or more cytotoxic agents, including one or more clastogenic compounds and/or one or more non-clastogenic chemotherapeutic compounds, and/or one or more sources of ionizing radiation, wherein the cancer cell is susceptible to growth and/or survival inhibition by the cytotoxic agents.

Within another embodiment, the present disclosure provides methods for treating a patient having a cancer that exhibits reduced MRN complex formation and/or functionality, by administering to the patient one or more cytotoxic agents, including one or more clastogenic compounds and/or one or more non-clastogenic chemotherapeutic compounds, and/or one or more sources of ionizing radiation, and/or a composition comprising one or more cytotoxic agents, wherein the cancer is susceptible to growth and/or survival inhibition by the cytotoxic agent. Related aspects of this embodiment employ a therapeutic regimen wherein one or more cytotoxic agents are administered prior to, simultaneously with, or following the administration of one or more additional cytotoxic agents and/or one or more non-cytotoxic therapeutic agents.

Within certain aspects of these methods, the cancer is selected from the group consisting of a breast cancer, a hormone-negative breast cancer (HNBC), a triple-negative breast cancer (TNBC), a colorectal cancer, an urothelial cancer, and a non-breast/colorectal/urothelial cancer, wherein the cancer exhibits reduced MRN complex formation and/or functionality.

Within other embodiments, the present disclosure provides compounds, compositions, and therapeutic regimens for inhibiting the growth and/or survival of a cancer cell exhibiting reduced MRN complex formation and/or functionality as well as methods for identifying compounds, compositions, and therapeutic regimens that inhibit the growth and/or survival of a cell exhibiting reduced MRN complex formation and/or functionality.

### BRIEF DESCRIPTION OF THE DRAWINGS

FIG. 1 is a graph showing disease-free survival of triple negative breast cancer (TNBC) patients treated with neo-adjuvant chemotherapy who either achieved a pathological complete response (pCR) or did not achieve a pathological complete response (no pCR). Taken from von Minckwitz et al., JCO 30:1796-1804 (2013).

FIGS. 2A and 2B are photographic images of exemplary MRE11 immunohistochemistry (IHC) analyses of triple-negative breast cancer (TNBC) tissue microarrays with a representative breast tumor having a normal level of the MRE11 complex (FIGS. 2A-i and 2B-i) or an abnormally low level of the MRE11 complex (FIGS. 2A-ii and 2B-ii). Scale bars=100 μm. These data demonstrate that low MRE11 staining (FIG. 2A) and low NBS1 staining (FIG. 2B) correlate with improved overall survival in TNBC.

FIG. 3 is a bar graph showing the distribution of tumor stage (left panel) and nodal stage (right panel) in breast tumors expressing normal levels of the MRE11 complex vs. low levels of the MRE11 complex.

FIG. 4 is a Kaplan-Meier plot of percent overall survival showing a statistically-significant (p=0.032) increase in survival rate for patients (n=16) having hormone negative cancer (e.g., triple negative breast cancer; TNBC) exhibiting a reduced level of Mre11 expression (MRE11 Complex Low) as compared to patients (n=155) having hormone negative cancer (e.g., triple negative breast cancer) exhibiting a normal level of Mre11 expression (MRE11 Complex Normal) subjected to a treatment regimen that included surgery followed by a combination of chemotherapy and radiotherapy.

FIG. 5 is a graph of proportion colony formation vs. dose (Gy) of the DNA damaging agent ionizing radiation (IR), which shows that murine embryonic fibroblast cells that express a low level of Mre11 (Mre11-impaired; Mre11^{ATLD1/ATLD1}) exhibit enhanced susceptibility to IR exposure as compared to murine embryonic fibroblast cells that express a normal level of Mre11 (WT). All data points represent mean values of triplicate samples, and error bars indicate the SEM. **=P<0.01 according to unpaired t tests.

FIG. 6 is a graph of proportion colony formation vs. concentration (μM) of the DNA damaging agent Mechlorethamine (HN2), an alkylating agent that is similar to cyclophosphamide, which shows that murine embryonic fibroblast cells that express a low level of Mre11 (Mre11-impaired; Mre11^{ATLD1/ATLD1}) exhibit enhanced susceptibility to Mechlorethamine as compared to murine embryonic fibroblast cells that express a normal level of Mre11 (WT). All data points represent mean values of triplicate samples, and error bars indicate the SEM. ***=P<0.001 according to unpaired t tests.

FIG. 7 is a graph of proportion colony formation vs. concentration (μg/ml) of the DNA damaging agent Adriamycin (ADR), which shows that murine embryonic fibroblast cells that express a low level of Mre11 (Mre11-impaired; Mre11^{ATLD1/ATLD1}) exhibit an equivalent response (i.e., no difference in susceptibility) to Adriamycin as compared to murine embryonic fibroblast cells that express a normal level of Mre11 (WT). All data points represent mean values of triplicate samples, and error bars indicate the SEM.

### DETAILED DESCRIPTION

The present disclosure is based upon the discovery that certain non-germline changes and mutations, including epigenetic changes and somatic mutations, in genes expressing and/or regulating the expression of the proteins MRE11, RAD50, and NBS1 can reduce MRN complex formation and/or functionality in cancer cells. Moreover, as disclosed herein, it was further discovered that certain cancers having reduced MRN complex formation and/or functionality exhibit increased sensitivity to certain cytotoxic agents, such as certain clastogenic and other chemotherapeutic compounds and radiation sources, as compared to non-cancer cells exhibiting normal or wild-type MRN complex formation and/or functionality.

Cancers, including breast cancers, including hormone-negative breast cancers (HNBCs) and triple-negative breast cancers (TNBCs), colorectal cancers, urothelial cancers; and other cancers that also exhibit reduced MRN complex formation and/or functionality are, as a consequence of reduced MRN complex formation, more susceptible to treatment with such cytotoxic agents as are cancers that exhibit normal or wild-type MRN complex formation and/or functionality. Due to the increased susceptibility of such cancers to cytotoxic agents, these cancers may be advantageously treated (1) by the preferential administration of one or more cytotoxic agents (including clastogenic compounds, non-clastogenic chemotherapeutic compounds, and/or sources of ionizing radiation), the mode of action of which cytotoxic agents is associated with one or more functional activities of the MRN complex and/or (2) by administration of lower doses of such cytotoxic agents thereby reducing the toxicity that is frequently attributed to therapeutic regimens that employ higher doses of those cytotoxic agents.

Based upon these and other discoveries, which are described in detail herein, the present disclosure provides:
(1) methods for identifying a cancer cell exhibiting reduced MRN complex formation and/or functionality, which cancer cell is, as a consequence of reduced MRN complex formation and/or functionality, sensitive to growth and/or survival inhibition by one or more cytotoxic agents, such as one or more clastogenic or other chemotherapeutic compounds and/or one or more sources of ionizing radiation;
(2) methods for identifying a patient having a cancer exhibiting reduced MRN complex formation and/or functionality, which cancer comprises one or more cells that, as a consequence of reduced MRN complex formation and/or functionality, is sensitive to growth and/or survival inhibition by one or more cytotoxic agents, such as one or more clastogenic or other chemotherapeutic compounds and/or one or more sources of ionizing radiation;
(3) methods for inhibiting the growth and/or survival of a cancer cell that exhibits reduced MRN complex formation and/or functionality, which cancer cell, as a consequence of reduced MRN complex formation and/or functionality, is susceptible to or becomes more susceptible to growth and/or survival inhibition by contact with one or more cytotoxic agents, such as one or more clastogenic or other chemotherapeutic compounds and/or one or more sources of ionizing radiation;
(4) methods for treating a patient having a cancer that exhibits reduced MRN complex formation and/or functionality, which cancer comprises one or more cells that, as a consequence of reduced MRN complex formation and/or functionality, is susceptible to or becomes more susceptible to treatment by the administration of: (a) one or more cytotoxic agents, such as one or more clastogenic or other chemotherapeutic compounds and/or one or more source of ionizing radiation; (b) a composition comprising one or more cytotoxic agents, such as one or more clastogenic or other chemotherapeutic compounds and/or one or more sources of ionizing radiation; and/or (c) one or more cytotoxic agents, such as one or more clastogenic or other chemotherapeutic compounds and/or one or more sources of ionizing radiation, in combination with and/or independently from the administration of (i) one or more additional cytotoxic agents, such as one or more additional clastogenic or other chemotherapeutic compounds and/or one or more additional sources of ionizing radiation and/or (ii) one or more non-cytotoxic therapeutic agents;
(5) compounds, compositions, and therapeutic regimens for inhibiting the growth and/or survival of a cancer cell exhibiting reduced MRN complex formation and/or functionality, which compounds, compositions, and therapeutic regimens include one or more cytotoxic agents, such as one or more clastogenic or other chemotherapeutic compounds and/or one or more sources of ionizing radiation; and
(6) methods for identifying compounds, compositions, and therapeutic regimens that inhibit the growth and/or survival of a cell exhibiting reduced MRN complex formation and/or functionality, including methods for identifying one or more cytotoxic agents, such as one or more clastogenic or other chemotherapeutic compounds and/or one or more sources of ionizing radiation that inhibit the growth and/or survival of a cell exhibiting reduced MRN complex formation and/or functionality and/or for identifying compositions and/or therapeutic regimen that contain and/or employ such cytotoxic agents.

The compounds, compositions, and methods provided herein may be employed in the treatment of certain cancers, including certain breast cancers, in particular HNBCs and TNBCs, as well as certain colorectal, urothelial, and other cancers that exhibit reduced MRN complex formation and/or function and, as a consequence of reduced MRN complex formation and/or function, also exhibit an enhanced sensitivity to growth and/or survival inhibition by certain cytotoxic agents.

These and other aspects of the present disclosure can be better understood by reference to the following non-limiting definitions.

### DEFINITIONS

As used herein, the terms “hormone-negative breast cancer” and “HNBC) refer to breast cancers that are negative for estrogen receptors (ER⁻), progesterone receptors (PR⁻), or both. Thus, HNBCs are ER⁻, PR⁻, or ER⁻PR⁻. As used herein, the terms “triple-negative breast cancer” and “TNBC” refer to those breast cancers that are, in addition to being ER⁻PR⁻ HNBCs, are also In some embodiments, hormone negative breast cancers are “triple negative” breast cancers, which, in addition to being ER⁻ and PR⁻ negative, are also negative for the HER2/neu translocation.

As used herein, the term “diagnosed” refers to a determination that has been made that the cancer is, for example, a breast cancer, a colorectal cancer, a urothelial cancer, or other cancer that exhibits reduced MRN complex formation and/or functionality. A diagnosis may be made prior to (on a different sample) performing the present methods for inhibiting the growth and/or survival of a breast cancer, a colorectal cancer, a urothelial cancer, or other cancer that exhibits reduced MRN complex formation and/or functionality or a diagnosis may be made in conjunction (i.e., either concurrently or sequentially) with the present methods for inhibiting the growth and/or survival of a breast cancer, a colorectal cancer, a urothelial cancer, or other cancer that exhibits reduced MRN complex formation and/or functionality.

As used herein, the term “identifying” refers to an initial determination that a cancer cell exhibits reduced MRN complex formation and/or functionality and/or that a cancer cell exhibits enhanced susceptibility to a cytotoxic agent. “Identifying” does not determine the selection of a final medical treatment regimen, but may be used by a skilled clinician in designing and/or selecting such a treatment regimen.

As used herein, the terms “homologous recombination” and “HR” refer to a type of genetic recombination in which nucleotide sequences are exchanged between two similar or identical molecules of DNA. Homologous recombination is most widely used by cells to accurately repair DNA double-strand breaks. Although homologous recombination varies widely among different cell types, most forms involve the same basic steps. After a double-strand break occurs, sections of DNA around the 5′ ends of the break are cut away (resection). In a strand invasion step, an overhanging 3′ end of the broken DNA molecule then “invades” a similar or identical DNA molecule that is not broken. After strand invasion, one or two cross-shaped structures called Holliday junctions connect the two DNA molecules. Depending upon how the two junctions are cut by enzymes, the type of homologous recombination that occurs in meiosis results in either chromosomal crossover or non-crossover. Homologous recombination that occurs during DNA repair tends to yield non-crossover products thereby restoring the damaged DNA molecule as it existed before the double-strand break. Dysfunction in the cellular machinery responsible for homologous recombination has been strongly associated with increased susceptibility to several types of cancer.

As used herein, the term “MRN complex” refers to a protein complex that includes the MRE11, RAD50, and NBS1 proteins. In eukaryotes, the MRN complex plays an important role in the initial processing of double-strand DNA breaks prior to repair by homologous recombination or non-homologous end joining. The MRN complex binds to double-strand breaks both in vitro and in vivo and may serve to tether broken ends prior to repair by non-homologous end joining or to initiate resection prior to repair by homologous recombination. The MRN complex also participates in activating the checkpoint serine/threonine protein kinase ataxia telangiectasia mutated (ATM) in response to DNA damage. Lee and Paull, Science 304(5667):93-6 (2004) and Lee and Paull, Science 308(5721):551-4 (2005).

Production of short single-strand oligonucleotides by Mre11 endonuclease activity has been implicated in ATM activation by the MRN complex. Jazayeri et al., EMBO J. 27(14):1953-1962 (2008). During both meiotic and mitotic repair, the MRN complex influences DSB repair structurally, by forming a bridge between the participating DNA molecules, and enzymatically, by promoting the resection of DSB ends. Mimitou and Symington, DNA Repair (Amst.) 8:983-995 (2009). The MRN complex is highly conserved, with readily identifiable orthologues of MRE11 and RAD50 evident in eubacterial, archaeal and eukaryal genomes. NBS1 appears to be confined to eukarya and, within that domain, is somewhat less conserved than MRE11 or RAD50.

As used herein, the term “MRE11” refers to a nuclear protein that is involved in homologous recombination, telomere length maintenance, and DNA double-strand break repair. MRE11 has 3′ to 5′ exonuclease activity and an endonuclease activity. MRE11 forms a complex with RAD50, which is required for non-homologous joining of DNA ends and possesses increased single-stranded DNA endonuclease and 3′ to 5′ exonuclease activities. In addition to RAD50, MRE11 also interacts with NBS1 to form the MRN complex. The Mre11 gene has a pseudogene on chromosome 3. Four variants of Mre11 are presented herein as SEQ ID NOs 11-14 that encode the MRE11 isoforms presented herein as SEQ ID NOs 1-4, respectively.

As used herein, the term “RAD50” refers to a protein involved in DNA double-strand break repair. RAD50 is a member of the structural maintenance of chromosomes (SMC) family of proteins. Like other SMC proteins, RAD50 contains a long internal coiled-coil domain that folds back on itself, bringing the N- and C-termini together to form a globular ABC ATPase head domain. RAD50 can dimerize both through its head domain and through a zinc-binding dimerization motif at the opposite end of the coiled-coil known as the “zinc-hook”. Results from atomic force microscopy suggest that in free MRN complexes, the zinc-hooks of a single RAD50 dimer associate to form a closed loop, while the zinc-hooks snap apart upon binding DNA, adopting a conformation that is thought to enable zinc-hook-mediated tethering of broken DNA ends. RAD50 forms a complex with MRE11 and NBS1 to form the MRN complex, which binds to broken DNA ends and displays numerous enzymatic activities that are required for double-strand break repair by non-homologous end-joining, or by homologous recombination. Three variants of Rad50 are presented herein as SEQ ID NOs 15-17 that encode the RAD50 isoforms presented herein as SEQ ID NOs 5-7, respectively.

As used herein, the terms “NBS1” and “Nibrin” refer to a protein, which is a member of the MRN complex, and has a role in regulating MRN complex activity, including end-processing of both physiological and mutagenic DNA double strand breaks (DSBs). Cellular response is performed by damage sensors, effectors of lesion repair and signal transduction. The central role is carried out by ataxia telangiectasia mutated (ATM) transducing kinase by activating the DSB signaling cascade, phosphorylating downstream substrates such as histone H2AX and NBS1. NBS1 relocates to DSB sites by interaction of FHA/BRCT domains with phosphorylated histone H2AX. Once it interacts with NBS1 c-terminal MRE11-binding domain, MRE11 and RAD50 relocate from the cytoplasm to the nucleus and then to sites of DSBs where the MRN complex forms foci at the site of DNA damage. Three variants of Nbs1 are presented herein as SEQ ID NOs 18-20 that encode the NBS1 isoforms presented herein as SEQ ID NOs 8-10, respectively.

### Cytotoxic, Clastogenic, and Other Chemotherapeutic Agents

The present disclosure provides cytotoxic agents, compositions comprising cytotoxic agents, and therapeutic regimens employing one or more cytotoxic agents, either alone or in combination with one or more non-cytotoxic therapeutic agents. As described herein, it was found that certain cytotoxic agents are effective in the treatment of cancers having a reduced level of MRN complex formation and/or functionality, which cancers exhibit an enhanced susceptibility to spontaneously-formed double-strand break (DSB) formation as well as DSBs that are induced through contact with an agent that causes DSB formation. Thus, it was discovered as part of the present disclosure that cells, including cancer cells, having a reduced level of MRN complex formation and/or functionality exhibit an increased sensitivity relative to growth and/or survival inhibition by certain cytotoxic agents, such as certain clastogenic and other chemotherapeutic compounds and radiation sources, that promote or cause DSB formation or that interfere with DSB repair by inhibiting one or more component of the DDR network.

As used herein, the term “cytotoxic agent” refers broadly to clastogenic agents and non-clastogenic agents. Cytotoxic agents can be anti-neoplastic cytotoxic agents that, for the purposes of the present disclosure, include clastogenic anti-neoplastic agents and non-clastogenic anti-neoplastic agents, which can be used in or are conventionally used for the treatment of one or more cancers. “Cytotoxic agents” may be used in single-agent therapies or may be used in combination therapies that include: (i) two or more cytotoxic agents or (ii) one or more cytotoxic agents and one or more non-cytotoxic therapy, including, for example, a surgery and/or a naturopathic therapy.

Anti-neoplastic cytotoxic agents can be “chemotherapeutic agents” or “radiotherapeutic agents.” As used herein, the term “chemotherapeutic agent” is synonymous with the terms “anti-neoplastic cytotoxic compound” and “chemotherapeutic compound.” As used herein, the term “radiotherapeutic agents” is synonymous with “anti-neoplastic radiation sources,” which include “ionizing radiation sources” and “sources of ionizing radiation.”

The term “chemotherapeutic agents” broadly encompasses the following “anti-neoplastic cytotoxic compounds,” which are grouped by class of compound: (1) alkylating agents (e.g., cyclophosphamide, mechlorethamine, chlorambucil, methyl methanesulfonate (MMS), and melphalan); (2) anthracyclines (e.g., daunorubicin, doxorubicin (a/k/a adriamycin (ADR)), epirubicin, idarubicin, mitoxantrone, and valrubicin); (3) cytoskeletal disrupters (taxanes) (e.g., paclitaxel, docetaxel, and eribulin); (4) epothilones (e.g., ixabepilone); (5) histone deacetylase inhibitors (e.g., vorinostat and romidepsin); (6) topoisomerase I inhibitors (e.g., irinotecan, topotecan, camptothecin, and lamellarin D); (7) topoisomerase II inhibitors (e.g., etoposide, teniposide, and tafluposide); (8) kinase inhibitors (e.g., bortezomib, erlotinib, gefitinib, imatinib, vemurafenib, and vismodegib); (9) monoclonal antibodies (e.g., bevacizumab, cetuximab, ipilimumab, ofatumumab, ocrelizumab, panitumab, and rituximab); (10) nucleotide analogs and precursor analogs (e.g., azacitidine, azathioprine, capecitabine, cytarabine, doxifluridine, fluorouracil, gemcitabine, hydroxyurea, mercaptopurine, methotrexate, and thioguanine); (11) peptide antibiotics (e.g., bleomycin and actinomycin); (12) cross-linking agents (e.g., carboplatin, cisplatin, oxaliplatin, and mitomycin C); (13) retinoids (e.g., tretinoin, alitretinoin, and bexarotene); and (14) vinca alkaloids and derivatives (e.g., vinblastine, vincristine, vindesine, and vinorelbine).

As used herein, “Chemotherapeutic agents” and “chemotherapeutic compounds” include anti-neoplastic compounds that are either clastogenic compounds or non-clastogenic compounds, and “radiotherapeutic agents” is synonymous with “anti-neoplastic radiation sources,” which include “ionizing radiation sources” and “sources of ionizing radiation” that are “clastogenic radiation sources.”

For clarity, when used herein in reference to the presently-disclosed compositions and methods for the treatment of cancer, the term “clastogenic agent” includes: (i) “clastogenic chemotherapeutic agents,” which are “anti-neoplastic clastogenic compounds,” and (ii) “clastogenic radiotherapeutic agents,” which are “ionizing radiation sources” or “sources of ionizing radiation.” Similarly, when used herein in reference to the presently-disclosed compositions and methods for the treatment of cancer, the term “non-clastogenic agent” refers to “non-clastogenic chemotherapeutic agents,” which are “anti-neoplastic clastogenic compounds.” Thus, the terms “non-clastogenic agent” and “non-clastogenic compound” are synonymous and are used interchangeably herein. Unless the context suggests otherwise, the clastogenic and non-clastogenic agents described herein have in common the property that they can be used in or are conventionally used for the treatment of one or more cancers.

Thus, as used herein, “clastogenic agents” are a subset of “cytotoxic agents,” which include clastogenic compounds and sources of ionizing radiation (which are also clastogenic) that induce double-strand breaks in DNA, primarily chromosomal DNA, thereby causing sections of chromosomes to be deleted, added, or rearranged. See, for example, Stracker and Petrini, Nature Rev. Mol. Cell Bio. 12:90-103 (2011) and Rosefort et al., Mutagenesis 19:277-284 (2004). “Clastogenic agents” can also cause sister chromatid exchanges, which are homologous chromatid strand interchanges and reunions that occur during DNA replication. Call et al., Mutat. Res. 160(3):249-257 (1986).

Clastogenic agents can induce double-strand breaks in a cell's chromosomal DNA that, if not repaired, inhibit the growth and/or survival of the cell. As described herein, it was found as part of the present disclosure that certain, but not all, clastogenic agents, are effective in inhibiting the growth and/or survival of a cell, including a cancer cell, having a reduced level of MRN complex formation and/or functionality and, as a consequence, a reduced capacity to repair chromosomal double-strand breaks induced by those clastogenic agents.

In one aspect, “clastogenic agents” include “sources of ionizing radiation (IR)” that are well known and are readily available in the art such as, for example, radiation derived from radioactive elements such as radon, uranium, and hydrogen; the decay products of radiation sources, including radioactive elements; and high energy particles (e.g., high linear-energy-transfer (LET) charged-particles) emitted by radiation sources. Ionizing radiation (IR) sources include X-rays and gamma (γ) rays, which are high frequency, high energy sources of electromagnetic radiation that are emitted from ⁶⁰Co or ¹³⁷Cs, ⁵⁶Fe, and ¹²C ions, and neutrons.

In another aspect, “clastogenic agents” include “clastogenic compounds” that are well known and are readily available in the art such as, for example, acridine yellow; benzene; ethylene oxide; arsenic; phosphine; mimosine; 5-azacytidine; 9,10-dimethyl-1,2-benzanthracene (DMBA); vincristine; topoisomerase I inhibitors (e.g., irinotecan, topotecan, camptothecin, and lamellarin D); topoisomerase II inhibitors (e.g., chloroquine, sodium azide, A-74932, etoposide (VP-16), teniposide, doxorubicin, daunorubicin, mitoxantrone, amsacrine, ellipticines, aurintricarboxylic acid, and the cannabidiol based quinolone HU-331); alkylating agents such as cyclophosphamide (CP); methyl methanesulfonate (MMS); mitomycin C (MMC); cisplatin; and adriamycin (ADR). “Clastogenic compounds” are available from Sigma Chem. Co. (St. Louis, Mo.) and are reviewed in Custer and Sweder, Curr. Drug. Metab. 9(9):978-985 (2008).

The “clastogenic compounds” and other “clastogenic agents” presented herein are intended for use in the compositions and methods of the disclosure. In particular, such “clastogenic compounds” and other “clastogenic agents” may be used in one or more “clastogenic cancer therapies,” which term, as used herein, refers to those cancer therapies that rely on a clastogen, or DNA break-inducing agent, to target proliferating cells in tumors as defined, for example, in Stracker and Petrini, Nat. Rev. Mol. Cell. Bio. 12:90-103 (2011). It will be understood that certain of these compounds, in particular acridine yellow, benzene, ethylene oxide, phosphine, mimosine, 5-azacytidine 9,10-dimethyl-1,2-benzanthracene (DMBA), while clastogenic, are not intended for in vivo administration to humans and, therefore, are not contemplated for use in the compositions and methods for treatment that are disclosed herein. These compounds may find use, however, in in vitro and ex vivo applications as will be understood by those of skill in the art.

As disclosed herein, it was discovered that, while certain cytotoxic agents, including certain clastogenic agents, such as the DNA damaging agents ionizing radiation (IR) and mechlorethamine (HN2), are effective in inhibiting the growth and/or survival of a cell having a reduced level of MRN complex formation and/or functionality, some cytotoxic agents, including some clastogenic agents, such as the DNA damaging agent doxorubicin (a/k/a adriamycin (ADR)), an anthracycline, had no effect on the growth and/or survival inhibition in a cell having a reduced level of MRN complex formation and/or functionality.

Based, in part, upon this observation, it is further contemplated that other cytotoxic agents including, for example, the cytoskeletal (microtubule) inhibitors paclitaxel and docetaxel will similarly have no effect on the growth and/or survival inhibition in a cell having a reduced level of MRN complex formation and/or functionality.

According to the present disclosure, it is contemplated that cytotoxic compounds, including clastogenic compounds, which exhibit enhanced growth and/or survival inhibition in a cell having a reduced level of MRN complex formation and/or functionality as compared to a cell having a normal or wild-type level of MRN complex formation and/or functionality include alkylating agents, topoisomerase I inhibitors, and cross-linking agents. Suitable alkylating agents for inhibiting growth and/or survival inhibition in a cell having a reduced level of MRN complex formation and/or functionality include cyclophosphamide, mechlorethamine, chlorambucil, methyl methane sulfonate, and melphalan. Suitable topoisomerase I inhibitors for inhibiting growth and/or survival inhibition in a cell having a reduced level of MRN complex formation and/or functionality include irinotecan, topotecan, camptothecin, and lamellarin D. Suitable cross-linking agents for inhibiting growth and/or survival inhibition in a cell having a reduced level of MRN complex formation and/or functionality include the mitomycin C and the platinum-based agents carboplatin, cisplatin, and oxaliplatin.

Similarly, ionizing radiation exemplifies a clastogenic agent that exhibits enhanced growth and/or survival inhibition in a cell having a reduced level of MRN complex formation and/or functionality as compared to a cell having a normal or wild-type level of MRN complex formation and/or functionality.

In a related aspect of the present disclosure, it was also found that compounds that reduce MRN complex formation, such as PARP inhibitors, ATM inhibitors, ATR inhibitors, DNA-PK inhibitors, Chk1 inhibitors, and homologous recombination inhibitors are also effective in inhibiting the growth and/or survival of a cell, in particular certain cancer cells such as breast cancer cells, including hormone-negative breast cancer cells (HNBCs) and triple-negative breast cancer cells (TNBCs), colorectal cancer cells, urothelial cancer cells; and other cancer cells as a result of PARP, ATM, ATR, DNA-PK, Chk1, and/or homologous recombination inhibitor-mediated reduction in MRN complex formation and/or functionality.

It is contemplated, therefore, that PARP, ATM, ATR, DNA-PK, Chk1, and/or homologous recombination inhibitors may also be employed in the presently-disclosed methods for the treatment of such cancers and that therapeutic regimens for the treatment of cancers may comprise the administration of one or more PARP, ATM, ATR, DNA-PK, Chk1, and/or homologous recombination inhibitors alone or in combination with one or more suitable clastogenic agent, such as a clastogenic compound (e.g., one or more alkylating agents, topoisomerase I inhibitors, and/or cross-linking agents) or a source of ionizing radiation, and/or one or more additional non-clastogenic therapeutic agent.

Without wishing to be limited by theory, it is believed that those clastogenic agents that generate DNA adducts that inhibit homologous recombination mediated by the MRN complex (such as cyclophosphamide, mechlorethamine, chlorambucil, methyl methanesulfonate (MMS), melphalan, and other alkylating agents; irinotecan, topotecan, camptothecin, lamellarin D and other topoisomerase inhibitors; cisplatin, carboplatin, oxaliplatin, mitomycin C, and other crosslinking agents; and ionizing radiation) include the clastogenic agents that are most effective in enhancing growth and/or survival inhibition in a cell having a reduced level of MRN complex formation and/or functionality as compared to a cell having a normal or wild-type level of MRN complex formation and/or functionality.

The present disclosure contemplates that cytotoxic agents, including alkylating agents (e.g., cyclophosphamide, mechlorethamine, chlorambucil, methyl methanesulfonate (MMS), and melphalan); topoisomerase I inhibitors (e.g., irnotecan, topotecan, camptothecin, lamellarin D); cross-linking agents (e.g., cisplatin, carboplatin, oxalplatin, and mitomycin C); nucleotide and precursor analogs (e.g., azacitidine, azathioprine, capecitabine, cytarabine, doxifluriding, fluorouracil (5-FU), gemcitabine, hydroxyurea, mercaptopurine, methotrexate, and thioguanine); and other DNA damage response (DDR) signaling and repair inhibitors (e.g., PARP inhibitors, ATM inhibitors, ATR inhibitors, DNA-PK inhibitors, Chk1 inhibitors, and homologous recombination inhibitors), will provide substantially advantageous growth and/or survival inhibition in a cell having a reduced level of MRN complex formation and/or functionality as compared to a cell having a normal or wild-type level of MRN complex formation and/or functionality.

In contrast, the present disclosure contemplates that cytotoxic agents, including anthracyclines (e.g., daunorubicin, doxorubicin (adriamycin), epirubicin, idarubicin, mitoxantrone, and valrubicin); cytoskeletal disrupters—taxanes (e.g., paclitaxel, docetaxel, and eribulin); epothilones (e.g., ixabepilone); and vinca alkaloids and derivatives (e.g., vinblastine, vincristine, vindesine, and vinorelbine), will not provide substantially advantageous growth and/or survival inhibition in a cell having a reduced level of MRN complex formation and/or functionality as compared to a cell having a normal or wild-type level of MRN complex formation and/or functionality.

Table 1 summarizes (1) those cytotoxic compounds to which cancer cells having reduced MRN complex formation/functionality exhibit, or are predicted to exhibit, enhanced sensitivity as compared to cancer cells having a normal or wild-type level of MRN complex formation and/or functionality and (2) those cytotoxic compounds to which cancer cells having reduced MRN complex formation/functionality do not exhibit, or are predicted to not exhibit, enhanced sensitivity as compared to cancer cells having a normal or wild-type level of MRN complex formation and/or functionality.

Thus, the compositions and methods disclosed herein preferentially employ one or more cytotoxic agents including alkylating agents, topoisomerase I inhibitors, cross-linking agents, nucleotide and precursor analogs, and other DNA damage response (DDR) signaling and repair inhibitors, which provide, or are expected to provide, substantially advantageous growth and/or survival inhibition in a cell having a reduced level of MRN complex formation and/or functionality as compared to a cell having a normal or wild-type level of MRN complex formation and/or functionality.

In contrast, the compositions and methods disclosed herein need not employ, and preferentially omit, cytotoxic agents including anthracyclines, cytoskeletal disrupters—taxanes, epothilones, and vinca alkaloids and derivatives, which do not, or are expected not to, provide substantially advantageous growth and/or survival inhibition in a cell having a reduced level of MRN complex formation and/or functionality as compared to a cell having a normal or wild-type level of MRN complex formation and/or functionality.

TABLE 1
Relative Sensitivity of Cancer Cells to Cytotoxic Compounds Based upon
Level of MRN Complex Formation and/or Functionality

| Cytotoxic Compounds to | Cytotoxic Compounds |
|---|---|
| which Cancer Cells Having | to which Cancer Cells Having |
| Reduced MRN Complex | Reduced MRN Complex |
| Formation/Functionality Exhibit | Formation/Functionality do not |
| Enhanced Sensitivity | Exhibit Enhanced Sensitivity |
| Alkylating Agents | Anthracyclines |
| Cyclophosphamide | Daunorubicin |
| Mechlorethamine | Doxorubicin |
| Chlorambucil | (a/k/a adriamycin) |
| methyl methanesulfonate (MMS) | Epirubicin |
| Melphalan | Idarubicin |
| | Mitoxantrone |
| | Valrubicin |
| Topoisomerase I Inhibitors | Cytoskeletal Disrupters (Taxanes) |
| Irinotecan | Paclitaxel |
| Topotecan | Docetaxel |
| Camptothecin | Eribulin |
| Lamellarin D | |
| Cross-linking Agents | Epothilones |
| Cisplatin | Ixabepilone |
| Carboplatin | |
| Oxalplatin | |
| Mitomycin C | |
| Nucleotide and Precursor Analogs | Vinca Alkaloids and Derivatives |
| Azacitidine | Vinblastine |
| Azathioprine | Vincristine |
| Capecitabine | Vindesine |
| Cytarabine | Vinorelbine |
| Doxifluridine | |
| 5-Fluorouracil (5-FU) | |
| Gemcitabine | |
| Hydroxyurea | |
| Mercaptopurine | |
| Methotrexate | |
| Thioguanine | |
| DDR Signaling and Repair Inhibitors | |
| PARP inhibitors | |
| ATM inhibitors | |
| ATR inhibitors | |
| DNA-PK inhibitors | |
| Chk1 inhibitors | |
| Homologous recombination inhibitors | |

### Methology for Screening Candidate Compounds for Clastogenic Activity

Methodology for screening candidate compounds for clastogenic activity include the nuclear foci assay systems, such as the γ-H2AX, Mre11 complex, MDC1, and 53BP1 nuclear foci assay systems, described in Maser et al., Mol. Cell. Biol. 17:6087-6096 (1997); Anderson et al., Mol. Cell. Biol. 21:1719-1729 (2001); Paull et al., Curr. Biol. 10:886-895 (2000); and Petrini and Stracker, Trends in Cell Biology 13(9):458-462 (2003) as well as chromosome break assay systems described in Theunssen and Petrini, Methods in Enzymol. 409:251-284 (2006).

Traditional assay systems for characterizing the clastogenic activity of candidate compounds are well known in the art and are described, for example, in Dertinger, U.S. Pat. Nos. 8,586,321 and 8,076,095 and references cited therein.

The in vivo micronucleus methodology can be employed to screen candidate compounds for clastogenic (chromosomal breaking) activity. Schmid, Mut. Res. 31:9 (1975); Salamone et al., Mut. Res. 74:347 (1980); Heddle et al., Mut. Res. 123:61(1983); Salamone and Heddle, Chem. Mut. (de Serres, ed., Plenum Press) 8, 111 (1983). The test is based on the observation that mitotic cells with chromatid breaks or chromatid exchanges exhibit disturbances in the anaphase distribution of their chromatin. After telophase, this displaced chromatin can be excluded from the nuclei of the daughter cells and is found in the cytoplasm as a micronucleus.

Blood cells provide a sensitive model for evaluating clastogenic events since the nucleus of the erythrocyte stem cell is expelled a few hours after the last mitosis yielding DNA deficient cells. Treatment with clastogens or spindle positions which cause chromosomal breaks in the stem cell result in the formation of easily detectable micronuclei (MNs) in these anucleated young polychromatic erythrocytes (PCEs). These young anucleated cells are still rich in RNA and, therefore, exhibit unique staining patterns that distinguishes them from the mature normochromatic erythrocytes (RBCs).

For example, when blood is stained with a metachromatic dye such as acridine orange (AO) (Hayashi, et al. Mut. Res. 120:241 (1983)), the DNA of a micronucleus exhibits a bright green-yellow fluorescence. In contrast the young RNA rich anucleated PCEs exhibit red fluorescence when stained with AO and excited with a 488 nm light source. The RNA rich polychromatic cells (PCEs) find their way into the blood stream and eventually complete their evolution to the RNA deficient and nonfluorescent normochromatic red blood cells—the mature RBCs. The brief existence of the PCE cells (about 48 hrs) has been used by those skilled in the art to define the time frame for the conventional micronucleus assay by counting only MN in the PCE population. The present disclosure offers a more flexible analysis timeframe which is not dependent upon the PCE population and allows for a choice of assay times ranging from hours to weeks. Although bone marrow was used in the original micronucleus assay, McGregor et al. (Environmental Mutagenesis 2,509 (1980)) demonstrated that the micronucleated PCEs and RBCs accumulate in peripheral blood of mice following treatment with a clastogen. Blood provides a good supply of test material for the micronucleus test. The spontaneous background level of aberrations in blood or bone marrow cells is usually quite low (i.e. about 2 MN/1000 PCEs). Clastogenic agents can cause an increase in the relative number of micronuclei present.

Flow cytometric methodology for analyzing the ability of potential clastogenic agents, including chemicals and radiation, to cause chromosomal breaks in mammalian cells, including micronucleated cells in blood and bone marrow preparations, are described in Tometsko, U.S. Pat. No. 5,229,265 and Dertinger et al., U.S. Pat. No. 6,100,038. See, also, MacGregor et al., Environ. Mutagen. 2:509-514 (1980) Schmid, Mutation Res. 31:9 (1975); Salamone et al., Mutation Res. 74:347 (1980); Heddle et al., Mutation Res. 123:61(1983); and Salamone and Heddle, Chemical Mutagens 8:111 (de Serres, ed., Plenum Press, 1983).

An in vivo micronucleus test, as performed in laboratory rodents, may be employed as a short-term system to screen chemicals for clastogenic (i.e., chromosome-breaking). The test is based upon the observation that mitotic cells with either chromatid breaks or dysfunctional spindle apparatus exhibit disturbances in the anaphase distribution of their chromatin. After telophase, displaced chromatin can be excluded from the nuclei of a daughter cell and present in the cytoplasm as a micronucleus.

Treatment with clastogens and/or spindle poisons that cause genotoxic damage to stem cells results in the formation of easily detectable micronuclei in young anucleated reticulocytes, which are rich in RNA and certain surface markers (e.g., CD71). With appropriate staining, those anucleated reticulocytes can be distinguished from mature normo-chromatic erythrocytes.

From the bone marrow, reticulocytes enter the bloodstream where they evolve into RNA-deficient normo-chromatic erythrocytes. By scoring micronuclei exclusively in a short-lived reticulocyte population, variation to micronuclei frequency can be attributed to a recent cell cycle, making this system amenable to acute exposure protocols.

Historically, micronucleus analyses are conducted in rat bone marrow as opposed to rat peripheral blood because the rat spleen is able to capture and remove circulating micronucleated erythrocytes from peripheral circulation. It has been demonstrated, however, that rat peripheral blood is suitable for micronucleus assessment when the analysis is restricted to the youngest of the immature RET population (i.e., Type I and II reticulocytes). The result is a sensitive index of genotoxicity and a candidate compound's clastogenicity. Wakata et al., Envir. Mol. Mutagen. 21:136-143 (1998).

Analogous to the restriction of microscopic analysis of micronucleus scoring to Type I and Type II RETs based on RNA content, flow cytometric analysis can be limited to the youngest fraction of RETs based on the level of certain cell surface markers (e.g., CD71 or transferrin receptor expression). Fluorescent antibodies directed against the transferrin receptor can be used to label the RET population. This immunofluorescent labeling procedure closely parallels the RET enumeration methods based on RNA content. Dertinger et al., Mut. Res. 371:283-292 (1996). Due to the direct relationship between RET maturity and CD-71 content, it is possible to characterize RETs according to age with this technique.

This modification to the traditional mouse assay, which results in sensitive indications of genotoxicity in a rat peripheral blood compartment, has important implications for human biomonitoring. The spleen of a rat behaves very similarly to that of a human with regard to the effective scavenging of circulating micronucleated erythrocytes. Therefore, as with a rat, it is important to limit human peripheral blood micronucleus analyses to the youngest RETs (unless the individual is splenectomized).

Rapid and accurate way to enumerate micronucleated erythrocytes in a total peripheral blood erythrocyte pool can be achieved by employing flow cytometric methodology. One such method is disclosed in U.S. Pat. No. 5,229,265. In a flow cytometric method, cells pass in single file through a laser beam where a cell's fluorescence and light scatter properties are determined. In contrast to manual methods where only 1000-2000 cells per sample are scored, flow cytometers permit processing rates in excess of 8,000 cells/second. By evaluating more cells, greater scoring accuracy can be achieved.

Classically, reticulocytes are divided into five populations which are defined by the staining pattern observed in the presence of RNA-precipitating dyes. Stains such as thiazole orange (Lee et al., Cytometry 7:508-516 (1986)) and acridine orange (Seligman et al., Am. J. Hematol. 14:57-66 (1983)) are widely employed. With respect to flow cytometry-based micronucleus assays, however, these and other RNA dyes are problematic. Since RNA dyes actually bind to DNA as well, overlapping signals tend to limit the resolution of micronucleated reticulocytes from micronucleated normo-chromatic erythrocytes.

A flow cytometric method utilizing a dual dye combination consisting of thiazole orange and Hoechst 33342 has been described. Grawe et al., Cytometry 13:750-758 (1992). Thiazole orange stains the RNA component of the reticulocyte population, and Hoechst dye is used to label micronuclei. The dissimilar wavelengths necessary for excitation of DNA and RNA dyes necessitates the use of a dual-laser flow cytometer.

Accordingly, there is a need in this art for a rapid, simple and accurate technique to determine the changes in the micronucleated cell populations in the blood and bone marrow cells caused by the action of genotoxic agents. Such a technique would desirably use reticulocyte and micronuclei-specific labels that are excited by a similar wavelength but exhibit significantly different emission spectra, thus enabling the use of a single-laser flow cytometer in a flow cytometric-based micronucleus assay.

### Compositions and Therapeutic Regimen Comprising One or More Cytotoxic Compounds

The present disclosure provides compositions and therapeutic regimen that comprise one or more cytotoxic compounds, which compositions and therapeutic regimen may be employed in methods for the treatment of cancers exhibiting a reduced level of MRN complex formation and/or functionality and, as a consequence thereof, an enhanced sensitivity to growth and/or survival inhibition by those cytotoxic agents.

As used herein, the term “therapeutic regimen” refers to the type of treatment (i.e., the specific cytotoxic agents, sources of ionizing radiation, and/or other therapeutics); the strength (i.e. dosage) of the treatment; and the frequency and/or duration of the treatment. In the case of a cancer sample that has reduced levels of MRN complex formation and/or functionality, the enhanced susceptibility of the cancer to the presently-disclosed cytotoxic agents indicates that preferential treatment with clastogenic agents the activity of which is associated with the MRN complex, or reduced dosages and/or less frequent treatment may be satisfactorily employed to minimize toxic side effects of those agents. The ultimate treatment protocol for an individual patient will be determined by and is within the skill and knowledge of clinicians who practice in the field of cancer therapies and will include a consideration of factors including, for example, a patient's family history, age, and overall health and fitness.

It was discovered, as part of the present disclosure, that certain cytotoxic agents may be advantageously employed in methods for inhibiting the growth and/or survival of cancer cells that exhibit reduced MRN complex formation and/or functionality and, as a consequence, a reduced capacity to repair double-strand DNA breaks. Cancer cells that exhibit reduced MRN complex formation and/or functionality also exhibit an increase in sensitivity, as compared to cells having normal MRN complexes, to agents that cause double-strand DNA breaks (such as clastogenic agents) and to agents that inhibit the repair of double-strand breaks (such as PARP, ATM, ATR, DNA-PK, Chk1, and/or homologous recombination inhibitors).

It was further discovered that cancer cells that exhibit reduced MRN complex formation and/or functionality do not, however, exhibit an increase in sensitivity to growth and/or survival inhibition by all clastogenic agents. For example, it was found that while cancer cells exhibiting reduced MRN complex formation and/or functionality exhibit increased sensitivity to growth and/or survival inhibition by clastogenic agents exemplified by alkylating agents such as cyclophosphamide and methymethanesulfate (MMS); topoisomerase I inhibitors such as irinotecan, topotecan, and campththecin; and cross-linking agents such as cisplatin, carboplatin, and mitomycin C, such cancer cell do not exhibit increased sensitivity to growth and/or survival inhibition by clastogenic agents such as the anthracyclines, including daunorubicin, doxorubicin (a/k/a adriamycin (ADR)), epirubicin, idarubicin, mitoxantrone, and valrubicin.

Moreover, it was further discovered that the growth and/or survival sensitivity of cancer cells that exhibit reduced MRN complex formation and/or functionality to clastogenic agents such as the alkylating agents cyclophosphamide and methymethanesulfate (MMS), the topoisomerase I inhibitors irinotecan, topotecan, and campththecin, and the cross-linking agents cisplatin, carboplatin, and mitomycin C, may be further enhanced when used in combination with certain inhibitors of DNA damage response (DDR) signaling and repair including, for example, one or more PARP inhibitors, one or more ATM inhibitors, one or more ATR inhibitors, one or more DNA-PK inhibitors, one or more Chk1 inhibitors, as well as one or more other inhibitors of homologous recombination DNA repair mechanisms.

In contrast, however, it was also discovered that the growth and/or survival sensitivity of cancer cells that exhibit reduced MRN complex formation and/or functionality to clastogenic agents such as the alkylating agents cyclophosphamide and methymethanesulfate (MMS), the topoisomerase I inhibitors irinotecan, topotecan, and campththecin, and the cross-linking agents cisplatin, carboplatin, and mitomycin C, is not further enhanced when used in combination with certain inhibitors, which are exemplified herein by paclitaxel, docetaxel, ixabapilone, and eribulin.

Based, in part, upon these discoveries, the present disclosure contemplates that therapeutic regimens for the treatment of cancers exhibiting reduced MRN complex formation and/or functionality will advantageously employ one or more clastogenic agent such as the alkylating agents cyclophosphamide and methymethanesulfate (MMS), the topoisomerase I inhibitors irinotecan, topotecan, and campththecin, and the cross-linking agents cisplatin, carboplatin, and mitomycin C alone or in combination with one or more inhibitors of DDR signaling and repair, such as, for example, one or more PARP inhibitors, one or more ATM inhibitors, one or more ATR inhibitors, one or more DNA-PK inhibitors, one or more Chk1 inhibitors, and/or one or more other inhibitors of homologous recombination.

Moreover, the present disclosure further contemplates that therapeutic regimens for the treatment of cancers exhibiting reduced MRN complex formation and/or functionality will generally not employ the administration of any of adriamycin, epirubicin, paclitaxel, docetaxel, ixabapilone, and/or eribulin because such compounds exhibit unfavorable levels of toxicity but do not exhibit increased efficacy against exhibiting reduced MRN complex formation and/or functionality.

Thus, it is contemplated that the potential advantage of reducing in vivo toxicity by reducing the dosage of compounds such as the alkylating agents cyclophosphamide and methymethanesulfate (MMS), the topoisomerase I inhibitors irinotecan, topotecan, and campththecin, and the cross-linking agents cisplatin, carboplatin, and mitomycin C is not shared by any of adriamycin, epirubicin, paclitaxel, docetaxel, ixabapilone, and/or eribulin. For a review of conventional therapeutic regimens for triple-negative breast cancers, see, for example, Joensuu and Gligorov, Annals of Oncology 23 (Supp. 6):vi40-vi45 (2012) and Gelmon et al., Annals of Oncology 23:2223-2234 (2012).

More specifically, the present disclosure contemplates particular advantages of increased efficacy and decreased toxicity with therapeutic regimens for the treatment of cancers exhibiting reduced MRN complex formation and/or functionality that employ, for example, (1) cyclophosphamide alone or in combination with one or more of methotrexate, 5-fluorouracil (5-FU), carboplatin, and/or cisplatin; (2) epirubicin alone or in combination with one or more of cyclophosphamide and/or 5-FU; (3) carboplatin and/or cisplatin alone or in combination with one or more of a PARP inhibitor, an ATM inhibitor, an ATR inhibitor, a DNA-PK inhibitor, a Chk1 inhibitors, and/or another inhibitors of homologous recombination.

Based, in part, upon this discovery, the present disclosure provides compositions and methods for the treatment of cancers exhibiting reduced MRN complex formation and/or functionality, which compositions and methods employ one or more clastogenic agents either alone or in combination with one or more other cancer treatments, such as surgeries, targeted therapies, and/or non-clastogenic cytotoxic agents, which cancer treatments may be performed prior to, in conjunction with, or following the methods of the present disclosure.

It will be understood that one aspect of the present disclosure is the application of certain clastogenic agents that induce a double-strand break in the DNA of a cancer cell exhibiting reduced MRN complex formation and/or functionality, which double-strand DNA break is repaired less efficiently, or cannot be repaired at all, in a cancer cell exhibiting reduced MRN complex formation and/or functionality. As disclosed herein, one or more of those clastogenic agents can be used in combination with one or more other agents, such as one or more PARP inhibitors and/or one or more Chk1 inhibitors, which further reduce double-strand DNA repair mediated by the MRN complex.

One or more cytotoxic compounds can be administered to a human patient by themselves or in compositions, such as pharmaceutical compositions, where they are mixed with suitable carriers or excipient(s) at doses to treat or ameliorate a cancer as described herein. Mixtures of these cytotoxic compounds can also be administered to a patient as a simple mixture or in suitable formulated compositions, including pharmaceutical compositions.

Compositions within the scope of this disclosure include compositions wherein the therapeutic agent is a cytotoxic compound in an amount effective to inhibit the growth and/or survival of a cancer cell in a patient. Determination of optimal ranges of effective amounts of each component is within the skill of the art. The effective dose is a function of a number of factors, including the specific cytotoxic compound, the presence of a prodrug, the patient and the clinical status of the latter.

Compositions comprising a cytotoxic compound may be administered parenterally. As used herein, the term “parenteral administration” refers to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, and intrasternal injection and infusion. Alternatively, or concurrently, administration may be orally.

The dosage administered will be dependent upon the age, health, and weight of the recipient, kind of concurrent treatment, if any, frequency of treatment, and the nature of the effect desired.

Compositions comprising a cytotoxic compound may, for example, be administered intravenously via an intravenous push or bolus. Alternatively, compositions comprising a cytotoxic compound may be administered via an intravenous infusion.

Suitable dosages for intravenous infusion of a composition comprising a cytotoxic compounds can be determined in reference to Joensuu and Gligorov, Annals of Oncology 23 (Supp. 6):vi40-vi45 (2012) and Gelmon et al., Annals of Oncology 23:2223-2234 (2012), which review of conventional therapeutic regimens for triple-negative breast cancers, see, for example.

Compositions comprising a cytotoxic compound generally include a therapeutically effective amount of a compound, and a pharmaceutically acceptable carrier. As used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skimmed milk, glycerol, propylene, glycol, water, ethanol and the like. The composition, if desired, can also contain minor amounts of wetting or emulsifying agents, or pH buffering agents.

These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. Such compositions will contain a therapeutically effective amount of the inhibitor, preferably in purified form, together with a suitable amount of carrier so as to provide the form for proper administration to the patient. The formulation should suit the mode of administration.

Compositions can be formulated in accordance with routine procedures as a pharmaceutical composition adapted for intravenous administration to a human. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The cytotoxic compounds disclosed herein can be formulated as neutral or salt forms. Pharmaceutically acceptable salts include those formed with anions such as those derived from hydrochloric, phosphoric, acetic, oxalic, tartaric acids, and the like, and those formed with cations such as those derived from sodium, potassium, ammonium, calcium, ferric hydroxides, isopropylamine, triethylamine, 2-ethylamino ethanol, histidine, procaine, and the like.

Many of the cytotoxic compounds of the present disclosure may be provided as salts with pharmaceutically compatible counterions (i.e., pharmaceutically acceptable salts). A “pharmaceutically acceptable salt” means any non-toxic salt that, upon administration to a recipient, is capable of providing, either directly or indirectly, a compound or a prodrug of a compound of the present disclosure. A “pharmaceutically acceptable counterion” is an ionic portion of a salt that is not toxic when released from the salt upon administration to a subject. Pharmaceutically compatible salts may be formed with many acids, including but not limited to hydrochloric, sulfuric, acetic, lactic, tartaric, malic, and succinic acids. Salts tend to be more soluble in water or other protic solvents than their corresponding free base forms. The present disclosure includes such salts.

When the therapeutic agents of the present disclosure are prepared for oral administration, they are preferably combined with a pharmaceutically acceptable carrier, diluent or excipient to form a pharmaceutical formulation, or unit dosage form. The total active ingredients in such formulations comprise from 0.1 to 99.9% by weight of the formulation. By “pharmaceutically acceptable” it is meant the carrier, diluent, excipient, and/or salt must be compatible with the other ingredients of the formulation, and not deleterious to the recipient thereof. The active ingredient for oral administration may be present as a powder or as granules; as a solution, a suspension or an emulsion; or in achievable base such as a synthetic resin for ingestion of the active ingredients from a chewing gum. The active ingredient may also be presented as a bolus, electuary or paste.

Pharmaceutical formulations containing the therapeutic agents of the present disclosure can be prepared by procedures known in the art using well known and readily available ingredients. For example, the agent can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

For example, tablets or caplets containing the agents of the present disclosure can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, and zinc stearate, and the like. Hard or soft gelatin capsules containing an agent of the present disclosure can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric coated caplets or tablets of an agent of the present disclosure are designed to resist disintegration in the stomach and dissolve in the more neutral to alkaline environment of the duodenum.

The therapeutic agents of the present disclosure can also be formulated as elixirs or solutions for convenient oral administration or as solutions appropriate for parenteral administration, for instance by intramuscular, subcutaneous or intravenous routes.

The pharmaceutical formulations of the therapeutic agents of the present disclosure can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol”, polyglycols and polyethylene glycols, C—C4 alkyl esters of short-chain acids, preferably ethyl or is ˜propyl lactate, fatty acid triglycerides such as the products marketed under the name “Miglyol”, isopropyl myristate, animal, mineral and vegetable oils and polysiloxanes.

The compositions according to the present disclosure can also contain thickening agents such as cellulose and/or cellulose derivatives. They can also contain gums such as xanthan, guar or carbo gum or gum arabic, or alternatively polyethylene glycols, bentones and montmorillonites, and the like.

It is possible to add, if necessary, an adjuvant chosen from antioxidants, surfactants, other preservatives, film-forming, keratolytic or comedolytic agents, perfumes and colorings. Also, other active ingredients may be added, whether for the conditions described or some other condition.

For example, among antioxidants, t-butylhydroquinone, butylated hydroxyanisole, butylated hydroxytoluene and a-tocopherol and its derivatives may be mentioned. The galenical forms chiefly conditioned for topical application take the form of creams, milks, gels, dispersion or microemulsions, lotions thickened to a greater or lesser extent, impregnated pads, ointments or sticks, or alternatively the form of aerosol formulations in spray or foam form or alternatively in the form of a cake of soap.

Additionally, the agents are well suited to formulation as sustained release dosage forms and the like. The formulations can be so constituted that they release the active ingredient only or preferably in a particular part of the intestinal or respiratory tract, possibly over a period of time. The coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, and the like.

The therapeutic agents of the present disclosure can be delivered via patches for transdermal administration. See U.S. Pat. No. 5,560,922 for examples of patches suitable for transdermal delivery of a therapeutic agent. Patches for transdermal delivery can comprise a backing layer and a polymer matrix which has dispersed or dissolved therein a therapeutic agent, along with one or more skin permeation enhancers. The backing layer can be made of any suitable material which is impermeable to the therapeutic agent. The backing layer serves as a protective cover for the matrix layer and provides also a support function. The backing can be formed so that it is essentially the same size layer as the polymer matrix or it can be of larger dimension so that it can extend beyond the side of the polymer matrix or overlay the side or sides of the polymer matrix and then can extend outwardly in a manner that the surface of the extension of the backing layer can be the base for an adhesive means. Alternatively, the polymer matrix can contain, or be formulated of, an adhesive polymer, such as polyacrylate or acrylate/vinyl acetate copolymer. For long-term applications it might be desirable to use microporous and/or breathable backing laminates, so hydration or maceration of the skin can be minimized.

Examples of materials suitable for making the backing layer are films of high and low density polyethylene, polypropylene, polyurethane, polyvinylchloride, polyesters such as poly(ethylene phthalate), metal foils, metal foil laminates of such suitable polymer films, and the like. Preferably, the materials used for the backing layer are laminates of such polymer films with a metal foil such as aluminum foil. In such laminates, a polymer film of the laminate will usually be in contact with the adhesive polymer matrix.

The backing layer can be any appropriate thickness which will provide the desired protective and support functions. A suitable thickness will be from about 10 to about 200 microns.

Generally, those polymers used to form the biologically acceptable adhesive polymer layer are those capable of forming shaped bodies, thin walls or coatings through which therapeutic agents can pass at a controlled rate. Suitable polymers are biologically and pharmaceutically compatible, nonallergenic and insoluble in and compatible with body fluids or tissues with which the device is contacted. The use of soluble polymers is to be avoided since dissolution or erosion of the matrix by skin moisture would affect the release rate of the therapeutic agents as well as the capability of the dosage unit to remain in place for convenience of removal.

Exemplary materials for fabricating the adhesive polymer layer include polyethylene, polypropylene, polyurethane, ethylene/propylene copolymers, ethylene/ethylacrylate copolymers, ethylene/vinyl acetate copolymers, silicone elastomers, especially the medical-grade polydimethylsiloxanes, neoprene rubber, polyisobutylene, polyacrylates, chlorinated polyethylene, polyvinyl chloride, vinyl chloride-vinyl acetate copolymer, crosslinked polymethacrylate polymers (hydrogel), polyvinylidene chloride, poly(ethylene terephthalate), butyl rubber, epichlorohydrin rubbers, ethylene vinyl alcohol copolymers, ethylene-vinyloxyethanol copolymers; silicone copolymers, for example, polysiloxane-polycarbonate copolymers, polysiloxanepolyethylene oxide copolymers, polysiloxane-polymethacrylate copolymers, polysiloxane-alkylene copolymers (e.g., polysiloxane-ethylene copolymers), polysiloxane-alkylenesilane copolymers (e.g., polysiloxane-ethylenesilane copolymers), and the like; cellulose polymers, for example methyl or ethyl cellulose, hydroxy propyl methyl cellulose, and cellulose esters; polycarbonates; polytetrafluoroethylene; and the like.

Preferably, a biologically acceptable adhesive polymer matrix should be selected from polymers with glass transition temperatures below room temperature. The polymer may, but need not necessarily, have a degree of crystallinity at room temperature. Cross-linking monomeric units or sites can be incorporated into such polymers. For example, cross-linking monomers can be incorporated into polyacrylate polymers, which provide sites for cross-linking the matrix after dispersing the therapeutic agent into the polymer. Known cross-linking monomers for polyacrylate polymers include polymethacrylic esters of polyols such as butylene diacrylate and dimethacrylate, trimethylol propane trimethacrylate and the like. Other monomers which provide such sites include allyl acrylate, allyl methacrylate, diallyl maleate and the like.

Preferably, a plasticizer and/or humectant is dispersed within the adhesive polymer matrix. Water-soluble polyols are generally suitable for this purpose. Incorporation of a humectant in the formulation allows the dosage unit to absorb moisture on the surface of skin which in turn helps to reduce skin irritation and to prevent the adhesive polymer layer of the delivery system from failing.

Therapeutic agents released from a transdermal delivery system must be capable of penetrating each layer of skin. In order to increase the rate of permeation of a therapeutic agent, a transdermal drug delivery system must be able in particular to increase the permeability of the outermost layer of skin, the stratum corneum, which provides the most resistance to the penetration of molecules. The fabrication of patches for transdermal delivery of therapeutic agents is well known to the art.

For administration to the upper (nasal) or lower respiratory tract by inhalation, the therapeutic agents of the present disclosure are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the therapeutic agent may be administered via nose drops, a liquid spray, such as via a plastic bottle atomizer or metered-dose inhaler. Typical of atomizers are the Mistometer (Wintrop) and the Medihaler (Riker).

The local delivery of the therapeutic agents of the present disclosure can also be by a variety of techniques which administer the agent at or near the site of disease. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

For topical administration, the therapeutic agents may be formulated as is known in the art for direct application to a target area. Conventional forms for this purpose include wound dressings, coated bandages or other polymer coverings, ointments, creams, lotions, pastes, jellies, sprays, and aerosols. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the present disclosure present in a topical formulation will depend on various factors, but generally will be from 0.01% to 95% of the total weight of the formulation, and typically 0.1-25% by weight.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic agent may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present disclosure in a suitable liquid carrier.

The formulations and compositions described herein may also contain other ingredients such as antimicrobial agents, or preservatives. Furthermore, the active ingredients may also be used in combination with other therapeutic agents, for example, bronchodilators.

### Methods for Identifying Cancers and Cancer Cells that are Sensitive to Cytotoxic Compound-Mediated Inhibition of Growth and/or Survival

As described in detail herein, cancers and cancer cells that exhibit a reduced level of MRN complex formation and/or functionality as compared to MRN complex formation and/or functionality in a normal or wild-type cell, also exhibit an enhanced sensitivity to the growth and/or survival inhibitory properties of certain cytotoxic agents, including certain cytotoxic compounds. Thus, cancers, including breast cancers, in particular HNBCs and TNBCs, as well as certain colorectal, urothelial, and other cancers, which exhibit reduced MRN complex formation and/or functionality are susceptible to treatment with the cytotoxic agents disclosed herein.

Reduced levels of MRN complex formation and/or functionality within a cancer cell can result from: (1) reduced expression of one or more of the Mre11, Rad50, and/or Nbs1 genes; (2) reduced levels of one or more of the MRE11, RAD50, and/or NBS1 proteins; and/or (3) a mutation, insertion, and/or deletion in an Mre11, Rad50, and/or Nbs1 gene, which mutation, insertion, and/or deletion, when expressed, reduces or eliminates one or more functions of an MRE11, RAD50, and/or NBS1 protein.

The method in this aspect of the disclosure is to identify a tissue sample of a hormone negative breast cancer tumor, a colorectal cancer tumor, or a urothelial cancer tumor. As used in this disclosure, the term “tissue sample” refers to a blood or plasma sample, an excised or aspirated sample of tumor tissue, and/or a sample that may be removed during a biopsy procedure or during a surgical tumor removal. The term “obtaining” refers to actual receipt of the tissue sample and need not be performed by a medical procedure. For example “obtaining” can be physical delivery or receipt of the tissue sample after it has been drawn, excised, or aspirated from the cancer tissue.

Tissue samples can be evaluated to determine an expression level of the MRN protein complex. As used in the present disclosure, the term “evaluating the tissue sample” is laboratory process where an expression level of the MRN complex is determined. It is believed that the expression level of the MRN complex is dependent upon the level of the component proteins (MRE11/RAD50/NBS1) of the complex. In particular, it is believed that an evaluation step of determining the amount of any of the individual component proteins individually will provide an indication of amount of complex. Therefore, this evaluation step may be performed by measuring the content of the complex itself and/or any of the individual component proteins.

The present methods may also include a comparison between the determined expression level of MRN protein complex and a relevant threshold level. In one embodiment, the relevant threshold level can be established by determining a specific population average/normal expression level of MRN complex in similar (e.g., breast, colon, lymph, blood, plasma, or marrow, etc.) but non-cancerous tissue (e.g., of a person of the same race, age, family etc.). In another embodiment the relevant threshold level can be ascertained by determining the MRN complex expression level in a sample from a similar but non-cancerous tissue from the patient. If an epigenetic change and/or somatic mutation has occurred within the sampled cancerous cells of the patient thereby decreasing expression of the MRN complex in the cancer sample, the decrease can be detected by comparison with the similar but non-cancerous tissue which contains normal or a threshold level of MRN complex expression.

It is noted that if the determined MRN complex protein and/or gene expression level of the cancerous and non-cancerous tissues from a given patient are both below a given population average for MRN complex, this might be indicative that the patient has a germline mutation in the gene(s) encoding the MRN complex or its component proteins. Such germline mutations are known to occur, for example, in ataxia telangiectasia disorder, it is believed that a mutation in the hMRE11 gene can be passed along from generation to generation. G. S Stewart et al., The DNA double-strand break repair gene hMRE11 is mutated in individuals with an ataxia-telangiectasia-like disorder. Cell 99:577 (1999). In such a scenario, clastogenic therapy may not a good choice for treating any cancer in the patient and could be particularly toxic.

The comparison step is performed to ascertain whether the determined MRN complex is below the relevant threshold value. In one embodiment, the comparison includes a statistical analyses between the determined MRN complex expression level and a relevant threshold. In this embodiment, the determined MRN complex expression level is considered “below” the relevant threshold where it is 1σ or more below, for example 2σ or more below, the relevant threshold value. In another embodiment, the comparison step includes a percentage analysis where the determined MRN complex expression level is considered “below” the relevant threshold where it is 25% or more, for example 50% or 75% or more below the relevant threshold value.

Where the determined expression level of MRN complex is below the relevant threshold level, the cancer can be identified as having enhanced susceptibility to cytotoxic agent-based therapy, including clastogenic therapy, and preferably treated with the therapy. While the cancer may be so identified, the ultimate selection and administration of any therapeutic treatment regimen for an individual patient will incorporate a clinician's understanding of other factors including, among others, family history, patient age, and overall health and fitness.

Thus, the methods disclosed herein for identifying a cancer cell or cancer tissue that is sensitive to growth or survival inhibition mediated by a cytotoxic compound employ methodology for:

(1) detecting the level of expression of one or more of the Mre11, Rad50, and/or Nbs1 genes in a cancer cell and comparing those levels of Mre11, Rad50, and/or Nbs1 gene expression to a predetermined threshold or to levels of Mre11, Rad50, and/or Nbs1 gene expression in a cell having wild-type or normal levels of Mre11, Rad50, and/or Nbs1 gene expression, determining whether the cancer cell exhibits reduced expression of one or more of the Mre11, Rad50, and/or Nbs1 genes relative to the threshold or cell having wild-type or normal gene expression levels, wherein reduced expression of one or more of the Mre11, Rad50, and/or Nbs1 genes relative to the threshold or cell having wild-type or normal gene expression levels is predictive of the sensitivity of the cancer cell to cytotoxic compound-mediated growth and/or survival inhibition;

(2) detecting the level of one or more of the MRE11, RAD50, and/or NBS1 proteins in a cancer cell and comparing those levels of MRE11, RAD50, and/or NBS1 proteins the levels of MRE11, RAD50, and/or NBS1 proteins to a predetermined threshold or to a cell having normal or wild-type levels of MRE11, RAD50, and/or NBS1 proteins thereby determining whether the cancer cell exhibits a reduced level of one or more of the MRE11, RAD50, and/or NBS1 proteins, wherein reduced expression of level of one or more of the MRE11, RAD50, and/or NBS1 proteins relative to the predetermined threshold or cell having normal or wild-type protein levels is predictive of the sensitivity of the cancer cell to cytotoxic compound-mediated growth and/or survival inhibition; and

(3) detecting a mutation, insertion, and/or deletion in an Mre11, Rad50, and/or Nbs1 gene, which mutation, insertion, and/or deletion is in a region of the Mre11, Rad50, and/or Nbs1 gene that, when the gene is expressed, reduces or eliminates a protein-protein contact between adjacent proteins or reduces or eliminates one or more function of an MRE11, RAD50, and/or NBS1 protein, wherein such a mutation, insertion, and/or deletion in one or more of the MRE11, RAD50, and/or NBS1 proteins reduces or eliminates MRN complex formation and/or functionality and wherein the reduction or elimination of the MRN complex formation and/or functionality is predictive of the sensitivity of the cancer cell to cytotoxic compound-mediated growth and/or survival inhibition.

### Methodology for Detecting Expression of Mre11, Rad50, and Nbs1 Genes

Reduced Mre11, Rad50, and/or Nbs1 gene expression can be determined by one or more methodologies that are well known in the art including, for example, microarray, quantitative PCR, including real-time-PCR (RT-PCR), and direct RNA sequencing. Each of the methodologies described herein for the detection of reduced Mre11, Rad50, and/or Nbs1 gene expression has in common the detection of a Mre11, Rad50, and/or Nbs1 polynucleotide via the amplification, hybridization, and/or sequencing of mRNA encoded by an Mre11, Rad50, and/or Nbs1 gene.

As used herein, the term “reduced gene expression,” in particular the term “reduced Mre11, Rad50, and/or Nbs1 gene expression,” refers to a level of gene expression that is less than about one-half, less than about one-third, less than about one-fifth, less than about one-tenth, less than about one-twentieth, or less than about one-fiftieth in a tissue sample or cell as compared to a control tissue or cell, which can be an internal or an external control tissue or cell.

Nucleotide sequences of cDNAs encoding the human isoforms of MRE1, RAD50, and NBS1 having the amino acid sequences presented in SEQ ID NOs: 1-10 are presented herein as SEQ ID NOs: 11-20. The cDNA sequences of SEQ ID NOs: 11-20 are predicted, degenerate nucleotide sequences, which were generated using the reverse translation algorithm “Bioinformatics Reverse Translation Tool” set to “Degenerate Mode.” (See, Table 2). “Bioinformatics Reverse Translation Tool” was developed by Greg Thatcher and is readily available from Thatcher Development Software, LLC.

TABLE 2
Bioinformatics Reverse Translation Tool Codon Codes for
Reverse Translation used in Degenerate Mode

| | | Reverse |
|---|---|---|
| | | Translation |
| | | used in |
| Amino Acid | Codons Encoding Amino Acid | Degenerate Mode |
| Ala | GCT, GCC, GCA, GCG | GCN |
| Cys | TGT, TGC | TGY |
| Asp | GAT, GAC | GAY |
| Glu | GAA, GAG | GAR |
| Phe | TTT, TTC | TTY |
| Gly | GGT, GGC, GGA, GGG | GGN |
| His | CAT, CAC | CAY |
| Ile | ATT, ATC, ATA | ATH |
| Lys | AAA, AAG | AAR |
| Leu | TTG, TTA, CTT, CTC, CTA, CTG | YTN |
| Met | ATG | ATG |
| Asn | AAT, AAC | AAY |
| Pro | CCT, CCC, CCA, CCG | CCN |
| Gln | CAA, CAG | CAR |
| Arg | CGT, CGC, CGA, CGG, AGA, AGG | MGN |
| Ser | TCT, TCC, TCA, TCG, AGT, AGC | WSN |
| Thr | ACT, ACC, ACA, ACG | CAN |
| Val | GTT, GTC, GTA, GTG | GTN |
| Trp | TGG | TGG |
| Tyr | TAT, TAC | TAY |
| STOP | TAA, TAG, TGA | TRR |
| (Termination) | | |

Nucleotide sequences of cDNAs encoding the human MRE1 isoform of GenBank Accession No. NP_{—}005581 (SEQ ID NO: 1) are presented herein as SEQ ID NO: 11). Nucleotide sequences of cDNAs encoding the human MRE1 isoform of GenBank Accession No. NP_{—}005582 (SEQ ID NO: 2) are presented herein as SEQ ID NO: 12). Nucleotide sequences of cDNAs encoding the human MRE1 isoform of GenBank Accession No. AAH05241 (SEQ ID NO: 3) are presented herein as SEQ ID NO: 13). Nucleotide sequences of cDNAs encoding the human MRE1 isoform of GenBank Accession No. AAC78721 (SEQ ID NO: 4) are presented herein as SEQ ID NO: 14).

Nucleotide sequences of cDNAs encoding the human RAD50 isoform of GenBank Accession No. AAB07119 (SEQ ID NO: 5) are presented herein as SEQ ID NO: 15). Nucleotide sequences of cDNAs encoding the human RAD50 isoform of GenBank Accession No. NP_{—}005723 (SEQ ID NO: 6) are presented herein as SEQ ID NO: 16). Nucleotide sequences of cDNAs encoding the human RAD50 isoform of GenBank Accession No. AAH62603 (SEQ ID NO: 7) are presented herein as SEQ ID NO: 17).

Nucleotide sequences of cDNAs encoding the human NBS1 isoform of GenBank Accession No. BAA28616 (SEQ ID NO: 8) are presented herein as SEQ ID NO: 18). Nucleotide sequences of cDNAs encoding the human NBS1 isoform of GenBank Accession No. AAC62232 (SEQ ID NO: 9) are presented herein as SEQ ID NO: 19). Nucleotide sequences of cDNAs encoding the human NBS1 isoform of GenBank Accession No. AAS59158 (SEQ ID NO: 10) are presented herein as SEQ ID NO: 20).

As used herein, the term “internal control” refers to a nucleotide sequence, typically a gene or genetic sequence, which does not exhibit reduced expression in a cancer tissue or cell as compared to a non-cancer tissue or cell. Thus, for example, an “internal control” can be used as a “negative control” for assessing whether an Mre11, Rad50, and/or Nbs1 gene exhibits reduced expression levels in a cancer tissue sample or cell without reference to a non-leukemia tissue sample or cell.

Suitable genes that can serve as “internal controls” include, for example and without limitation, β-actin, GAPDH, and cyclophilin. The levels of Mre11, Rad50, and/or Nbs1 gene expression and internal control gene expression (i.e., non-Mre11, Rad50, and/or Nbs1 gene expression) can be determined (e.g., by quantifying the number of Mre11, Rad50, and/or Nbs1 transcripts), a ratio of Mre11, Rad50, and/or Nbs1 and non-Mre11, Rad50, and/or Nbs1 gene expression can be derived, and the level of Mre11, Rad50, and/or Nbs1 gene expression within a given a cancer tissue sample or cell can be expressed in terms of the ratio of Mre11, Rad50, and/or Nbs1 and non-Mre11, Rad50, and/or Nbs1 gene expression, wherein a ratio less than a pre-determined threshold ratio indicates reduced Mre11, Rad50, and/or Nbs1 gene expression.

In contrast, as used herein, the term “external control” refers to a Mre11, Rad50, and/or Nbs1 gene or genetic sequence from a non-cancer tissue or cell, which Mre11, Rad50, and/or Nbs1 gene or genetic sequence does not exhibit reduced expression in the non-cancer tissue or cell but is being tested for reduced expression in a corresponding cancer tissue or cell. Thus, for example, an “external control” can be used as a “negative control” for assessing whether the Mre11, Rad50, and/or Nbs1 gene exhibits reduced expression levels in a cancer tissue sample or cell by comparing the level of expression (e.g., the number of mRNA transcripts) in a cancer tissue sample or cell to a corresponding non-cancer tissue sample, such as a tissue sample from a normal donor, or non-cancer cell.

Reduced Mre11, Rad50, and/or Nbs1 gene expression can also be assessed on the basis of the percentage or fraction of cancer cells relative to the total number of cells in a given tissue sample from a cancer patient. By this methodology, for example, the number of Mre11, Rad50, and/or Nbs1-associated transcripts in a cancer tissue sample can be quantified and multiplied by the inverse percentage or fraction of blasts in the cancer tissue sample. The resulting Mre11, Rad50, and/or Nbs1 transcript number can then be assessed relative to a threshold transcript number for gene expression and, based upon that assessment, the responsiveness of a cancer patient to a therapeutic regimen comprising the administration of a cytotoxic agent can be predicted. More specifically, by this methodology, a transcript number for Mre11, Rad50, and/or Nbs1 gene expression that is less than a threshold transcript number would be predictive of the therapeutic efficacy of such a cytotoxic agent.

Measurement of reduced Mre11, Rad50, and/or Nbs1 gene expression can, for example, be accomplished by (1) quantifying a Mre11, Rad50, and/or Nbs1 mRNA in a tissue sample from a cancer patient; (2) quantifying the level of the Mre11, Rad50, and/or Nbs1 mRNA in a tissue sample from a healthy control donor or from a normal cell (control) of the same patient; and (3) comparing the level of the Mre11, Rad50, and/or Nbs1 mRNA in the tissue sample from the cancer patient with the level of the Mre11, Rad50, and/or Nbs1 mRNA in the control. It will be understood that a reduced level of Mre11, Rad50, and/or Nbs1 mRNA in the cancer patient tissue sample as compared to Mre11, Rad50, and/or Nbs1 mRNA in the control donor tissue sample indicates the susceptibility of the cancer patient to treatment with a cytotoxic agent as described herein.

Alternatively, reduced Mre11, Rad50, and/or Nbs1 gene expression can be tested by (1) quantifying Mre11, Rad50, and/or Nbs1 mRNA levels in a tissue sample from a cancer patient; (2) quantifying the level of a non-Mre11, Rad50, and/or Nbs1 mRNA in the cancer patient tissue sample, such as, for example, GAPDH or actin; and (3) comparing the level of the Mre11, Rad50, and/or Nbs1 mRNA in the tissue sample from the cancer patient with the level of the non-Mre11, Rad50, and/or Nbs1 mRNA in the cancer patient tissue sample. It will be understood that an reduced level of the Mre11, Rad50, and/or Nbs1 mRNA in the cancer patient tissue sample as compared to the non-Mre11, Rad50, and/or Nbs1 mRNA in the cancer patient tissue sample indicates the susceptibility of the cancer patient to treatment with a cytotoxic agent as described herein.

Within certain aspects of these methods an Mre11, Rad50, and/or Nbs1 mRNA can be quantified by amplifying mRNA in a tissue sample, whether a cancer patient tissue sample or cell, a non-cancer tissue sample or cell from a cancer patient, or a tissue sample or cell from a non-cancer control donor, with a primer pair that is specific for Mre11, Rad50, and/or Nbs1 nucleotide sequences (see Table 4). Likewise, a non-Mre11, Rad50, and/or Nbs1 mRNA can be quantified by amplifying RNA in a tissue sample, whether a cancer patient tissue sample or cell, a non-cancer tissue sample or cell from a cancer patient, or a tissue sample or cell from a non-cancer control donor, with a primer pair that is specific for a non-Mre11, Rad50, and/or Nbs1 mRNA. A primer pair comprises a forward primer and a reverse primer, wherein the forward primer hybridizes toward the 5′ end of an mRNA and wherein said reverse primer hybridizes toward the 3′ end of the mRNA, whether the mRNA is an Mre11, Rad50, and/or Nbs1 RNA or a non-Mre11, Rad50, and/or Nbs1 mRNA.

Examples of nucleotide sequences for the Mre11, Rad50, and/or Nbs1 genes are presented in Table 4, as are the corresponding accession numbers and sequence identifiers.

In order to identify a patient tissue sample or cell that has reduced Mre11, Rad50, and/or Nbs1 gene expression, mRNA can be isolated from a cancer patient tissue sample or cell and from a non-cancer control tissue sample or cell, the level of expression of a given mRNA can be determined, and an assessment of reduced gene expression can be made by comparing the mRNA levels determined for a cancer patient tissue sample or cell and a non-cancer control tissue sample or cell.

Alternatively, a cancer patient tissue sample or cell that has reduced Mre11, Rad50, and/or Nbs1 gene expression can be identified by isolating mRNA from a cancer patient tissue sample or cell, determining the levels of a Mre11, Rad50, and/or Nbs1 mRNA and a control mRNA, and assessing reduced gene expression by comparing the Mre11, Rad50, and/or Nbs1 mRNA and control mRNA levels within the cancer tissue sample or cell to determine the ratio of mRNA expression, wherein an reduced ratio of Mre11, Rad50, and/or Nbs1 mRNA level relative to control mRNA level indicates an reduced level of Mre11, Rad50, and/or Nbs1 gene expression. As used in this context, a control mRNA refers to an mRNA from a gene that does not exhibit a reduced level of expression in a cancer tissue or cell. Suitable control mRNAs include, for example, β-actin, GAPDH, and cyclophilin.

Suitable cancer tissue samples include, for example, blood, lymph node, bone marrow, and/or tumor biopsy samples, including breast or colon tumor biopsy samples, from a cancer patient. Suitable non-cancer control tissue samples include, for example, blood, lymph node, and/or bone marrow samples from a non-cancer donor, such as a healthy, disease-free donor. It will be understood that, regardless of the precise nature or source of the donor tissue sample or cell, it is essential that the donor tissue or cell is known not to exhibit reduced expression of the Mre11, Rad50, and/or Nbs1 gene. Regardless of its source or identity, it will be understood that a suitable non-cancer control tissue sample or cell will be characterized by not exhibiting reduced levels of Mre11, Rad50, and/or Nbs1 gene.

Methodologies for quantifying gene expression levels that can be readily adapted to detecting reduced expression of Mre11, Rad50, and/or Nbs1 genes are now described in further detail.

1. Microarray Analysis

Reduced Mre11, Rad50, and/or Nbs1 gene expression can be detected and quantified by microarray analysis of RNA isolated from a cancer patient and/or control donor tissue sample- or cell. Due to limitations on its sensitivity, however, microarray methodology may not accurately determine the absolute tissue distribution of low abundance genes or may underestimate the degree of reduced Mre11, Rad50, and/or Nbs1 gene expression due to signal saturation. For those cells showing reduced Mre11, Rad50, and/or Nbs1 expression by microarray expression profiling, further analysis can be performed using one or more quantitative PCR methodology such as, for example, RT-PCR based on Taqman™ probe detection (Invitrogen Life Sciences, Carlsbad, Calif.), which provides a greater dynamic range of sensitivity.

Briefly, microarray analysis includes that PCR amplification of RNA extracted from a cancer patient or control donor tissue sample or cell with primer pairs that hybridize to coding sequences within an Mre11, Rad50, and/or Nbs1 gene and/or coding sequences within a non-Mre11, Rad50, and/or Nbs1 gene the expression of which is to be detected and/or quantified. PCR products are dotted onto slides in an array format, with each PCR product occupying a unique location in the array. The RNA is then reverse transcribed and fluorescent-labeled cDNA probes are generated. Microarrays are probed with the fluorescent-labeled cDNA probes, slides are scanned, and fluorescence intensity is measured. The level of fluorescence intensity correlates with hybridization intensity, which correlates with relative level of gene expression.

Mre11, Rad50, and/or Nbs1 gene expression analysis can be performed using a commercially available microarray (e.g., the U133A chip; Affymetrix, Santa Clara, Calif.) or using a custom microarray. Alternatively, reduced Mre11, Rad50, and/or Nbs1 gene expression can be detected using a Synteni microarray (Palo Alto, Calif.) according to the manufacturer's instructions and as described by Schena et al., Proc. Natl. Acad. Sci. U.S.A. 93:10614-10619 (1996) and Heller et al., Proc. Natl. Acad. Sci. U.S.A. 94:2150-2155 (1997). Microarray hybridization can be performed according to methodology described in Abraham et al., Blood 105:794-803 (2005).

Probe level data can be normalized using a commercial algorithm (e.g., the Affymetrix Microarray Suite 5.0 algorithm) or a custom algorithm. Mre11, Rad50, and/or Nbs1 gene expression intensity values as well as non-Mre11, Rad50, and/or Nbs1 gene expression intensity values can be log transformed, median centered, and/or analyzed using commercially available programs (e.g., GeneSpring 7.3.1 GX; Agilent Technologies, Santa Clara, Calif.) or a custom algorithm.

A number of factors can be used to assess the quality of the Mre11, Rad50, and/or Nbs1 gene expression analysis such as, for example, the GAPDH 3′:5′ ratio and the actin 3′:5′ ratio. Samples with poor quality results can be defined as having a GAPDH 3′:5′ ratio of greater than about 1.25 and/or an actin 3′:5′ ratio of greater than about 3.0.

Reduced Mre11, Rad50, and/or Nbs1 gene expression can be determined using Welch's ANOVA using variance computed by applying the cross-gene error model based on deviation from 1 available within GeneSpring. This can overcome a lack of replicates and variance associated with the individual samples and can be considered to be similar in principle to variance filtering. Unsupervised clustering can be done using a hierarchical agglomerative algorithm. Pearson's correlation coefficient and centroid linkage can be used as similarity and linkage methods, respectively.

To detect possible differences between samples, genes can be extracted from the dataset that had 1.5-fold difference in expression between individual samples and/or were statistically significant at a corrected P value of 0.05 by Student's t test with Benjamini-Hochberg multiple testing corrections. Differentially expressed genes can be assessed for Gene Ontology (GO) enrichment (e.g., using GeneSpring).

2. Quantitative PCR

Depending upon such factors as the relative number of cancer cells present in a cancer tissue sample and/or the level of Mre11, Rad50, and/or Nbs1 gene expression within each cancer cell within a tissue sample, it may be preferred to perform a quantitative PCR analysis to detect and/or quantify the level of Mre11, Rad50, and/or Nbs1 gene expression.

For example, at least two oligonucleotide primers can be employed in a PCR-based assay to amplify at least a portion of a Mre11, Rad50, and/or Nbs1 mRNA and/or a non-Mre11, Rad50, and/or Nbs1 mRNA, or a corresponding cDNA, which is derived from a cancer tissue sample or cell and/or a non-cancer control donor tissue sample or cell. At least one of the oligonucleotide primers is specific for, and hybridizes to, an mRNA that is encoded by an Mre11, Rad50, and/or Nbs1 gene. The amplified cDNA may, optionally, be subjected to a fractionation step such as, for example, gel electrophoresis prior to detection.

RT-PCR is a quantitative PCR methodology in which PCR amplification is performed in conjunction with reverse transcription. RNA is extracted from a tissue sample or cell, such as a blood, lymph node, bone marrow, and/or tumor biopsy sample, and is reverse transcribed to produce cDNA molecules. PCR amplification using at least one specific primer amplify the cDNA molecule, which may be separated and visualized using, for example, gel electrophoresis. Amplification may be performed on tissue samples or cells taken from a patient and from a control who is not afflicted with a cancer. The amplification reaction may be performed on several dilutions of cDNA spanning two orders of magnitude. A decrease in expression of at least about one-half, less than about one-third, less than about one-fifth, less than about one-tenth, less than about one-twentieth, or less than about one-fiftieth in a tissue sample or cell as compared to a control tissue or cell, which can be an internal or an external control tissue or cell in a cancer patient sample as compared to a non-cancer control donor sample generally constitutes a reduced level of gene expression.

As used herein, the term “amplification” refers to the production of multiple copies of a target nucleic acid that contains at least a portion of the intended specific target nucleic acid sequence. The multiple copies are referred to, interchangeably, as amplicons or amplification products. In certain aspects of the present disclosure, the amplified target contains less than the complete target mRNA sequence (i.e., spliced transcript of exons and flanking untranslated sequences) and/or target genomic sequence (including introns and/or exons). For example, specific amplicons may be produced by amplifying a portion of the target polynucleotide by using amplification primers that hybridize to, and initiate polymerization from, internal positions of the target polynucleotide. The amplified portion contains a detectable target sequence that may be detected using any of a variety of well-known methods.

Many well-known methods of nucleic acid amplification require thermocycling to alternately denature double-stranded nucleic acids and hybridize primers; however, other well-known methods of nucleic acid amplification are isothermal. The polymerase chain reaction (PCR; described in U.S. Pat. Nos. 4,683,195; 4,683,202; 4,800,159; 4,965,188) uses multiple cycles of denaturation, annealing of primer pairs to opposite strands, and primer extension to exponentially increase copy numbers of the target sequence. In a variation called RT-PCR, reverse transcriptase (RT) is used to make a complementary DNA (cDNA) from mRNA, and the cDNA is then amplified by PCR to produce multiple copies of DNA.

Mre11, Rad50, and/or Nbs1 gene expression may be further characterized or, alternatively, originally detected and/or quantified by employing the quantitative real-time PCR methodology. Gibson et al., Genome Research 6:995-1001 (1996) and Heid et al., Genome Research 6:986-994 (1996). Real-time PCR is a technique that evaluates the level of PCR product accumulation during the course of amplification. This technique permits quantitative evaluation of mRNA levels in multiple samples. By this methodology, a cancer tissue sample or cell may be tested along-side a corresponding non-Cancer control donor sample or cell and/or a panel of unrelated normal non-Cancer tissue samples or cells.

Real-time PCR may, for example, be performed either on the ABI 7700 Prism or on a GeneAmp® 5700 sequence detection system (Applied Biosystems, Foster City, Calif.). The 7700 system uses a forward and a reverse primer in combination with a specific probe with a 5′ fluorescent reporter dye at one end and a 3′ quencher dye at the other end (Taqman™). When real-time PCR is performed using Taq DNA polymerase with 5′-3′ nuclease activity, the probe is cleaved and begins to fluoresce allowing the reaction to be monitored by the increase in fluorescence (real-time). The 5700 system uses SYBR® green, a fluorescent dye that only binds to double stranded DNA, and the same forward and reverse primers as the 7700 instrument. Matching primers and fluorescent probes may be designed according to the primer express program (Applied Biosystems, Foster City, Calif.). Optimal concentrations of primers and probes are initially determined by those of ordinary skill in the art. Control (e.g., β-actin-specific) primers and probes may be obtained commercially from, for example, Perkin Elmer/Applied Biosystems (Foster City, Calif.).

To quantify the amount of Mre11, Rad50, and/or Nbs1 gene expression in a sample, a standard curve is generated using a plasmid containing the gene of interest. Standard curves are generated using the Ct values determined in the real-time PCR, which are related to the initial cDNA concentration used in the assay. Standard dilutions ranging from 10-10⁶ copies of the gene of interest are generally sufficient. In addition, a standard curve is generated for the control sample sequence. This permits standardization of initial RNA content of a cancer tissue sample or cell to the amount of a control tissue sample or cell for comparison purposes.

Total RNA may be extracted from cancer tissue samples or cells and non-cancer control tissue samples or cells using Trizol reagent as described herein. First strand synthesis may be carried out using 1-2 μg of total RNA with SuperScript II reverse transcriptase (Life Technologies, Carlsbad, Calif.) at 42° C. for one hour. cDNA may then be amplified by PCR with Mre11, Rad50, and/or Nbs1 gene-specific primers that are designed based upon the Mre11, Rad50, and/or Nbs1 nucleotide sequences presented in Table 4 or that are otherwise known and readily available to those skilled in the art.

To ensure the quantitative nature of the RT-PCR, a housekeeping gene, such as β-actin, can be used as an internal control for each of the Cancer patient and non-Cancer control donor tissue samples and/or cells examined. Serial dilutions of the first strand cDNAs are prepared and RT-PCR assays are performed using β-actin specific primers. A dilution is then chosen that enables the linear range amplification of the β-actin template and that is sensitive enough to reflect the differences in the initial copy numbers. Using these conditions, the β-actin levels are determined for each reverse transcription reaction from each tissue. DNA contamination is minimized by DNase treatment and by assuring a negative PCR result when using first strand cDNA that was prepared without adding reverse transcriptase.

In an exemplary RT-PCR reaction using the Dynabeads mRNA direct microkit (Invitrogen, Life Sciences Technologies, Carlsbad, Calif.), samples containing 10⁵ cells or less are tested in a total reaction volume of 30 μl with 14.25 μl H₂O; 1.5 μl BSA; 6 μl first strand buffer; 0.75 mL of 10 mM dNTP mix; 3 μl RNAsin; 3 μl 0.1 M dTT; and 1.5 μl Superscript II. The resulting solution is incubated for 1 hour at 42° C., diluted 1:5 in H₂O, heated at 80° C. for 2 min to detach cDNA from the beads, and immediately placed on MPS. The supernatant containing cDNA is transferred to a new tube and stored at −20° C.

3. RNA Sequencing

Reduced expression of a Mre11, Rad50, and/or Nbs1 gene can be determined by the direct sequencing of mRNA in a cancer patient tissue sample or cell and/or a non-Cancer donor control tissue sample or cell. Alternatively, reduced expression of the Mre11, Rad50, and/or Nbs1 gene can be determined following conversion of mRNA into cDNA by reverse transcription.

True Single Molecule Sequencing (tSMS™) and/or Direct RNA Sequencing (DRS™) are useful techniques for quantifying gene expression that can be readily adapted for detecting and quantifying the expression a Mre11, Rad50, and/or Nbs1 gene. These sequencing-by-synthesis technologies can be performed on mRNAs derived from a tissue sample or cell without the need for prior reverse transcription or PCR amplification.

Direct RNA sequencing technology (Helicos BioSciences Corporation, Cambridge, Mass.) and transcriptome profiling using single-molecule direct RNA sequencing are described in Ozsolak et al., Nature 461(7265):814-818 (2009) and Ozsolak and Milos, Methods Mol Biol 733:51-61 (2011). True Single Molecule and Direct RNA Sequencing technologies are further described in U.S. Patent Publication Nos. 2008/0081330, 2009/0163366, 2008/0213770, 2010/0184045, 2010/0173363, 2010/0227321, 2008/0213770, and 2008/0103058 as well as U.S. Pat. Nos. 7,666,593; 7,767,400; 7,501,245; and 7,593,109, each of which is hereby incorporated by reference in its entirety.

mRNAs encoded by a Mre11, Rad50, and/or Nbs1 gene can be directly sequenced by True Single Molecule and Direct RNA Sequencing technologies by utilizing specific sequencing primers that are designed based upon the Mre11, Rad50, and/or Nbs1 nucleotide sequences (e.g., as presented in Table 4 or which are otherwise known and readily available to those skilled in the art).

### Methodology for Detecting MRE11, RAD50, and NBS1 Protein Levels

Reduced MRE11, RAD50, and NBS1 protein levels can be determined by one or more methodologies that are well known in the art including, for example, immunohistochemical detection, immunofluorescent detection, immunoprecipitation, and western blotting detection. Each of the methodologies described herein for the detection of reduced MRE11, RAD50, and NBS1 protein levels has in common the detection of an MRE11, RAD50, and NBS1 protein level via the binding of an antibody to one or more of the MRE11, RAD50, and NBS1 proteins. Suitable antibodies and anti-sera for performing the presently disclosed methodology for detecting MRE11, RAD50, and NBS1 Protein Levels are described in U.S. Patent Publication No. 2003/0104427 and in Dolganov et al., Mol. Cell. Biol. 16(9):4832-4841 (1996) and are available from Novus Biologicals LLC (Littleton, Colo.).

As used herein, the term “reduced protein levels,” in particular the term “reduced MRE11, RAD50, and NBS1 protein levels,” refers to a cellular protein level that is less than about one-half, less than about one-third, less than about one-fifth, less than about one-tenth, less than about one-twentieth, or less than about one-fiftieth in a tissue sample or cell as compared to a control tissue or cell, which can be an internal or an external control tissue or cell.

The amino acid sequences of human MRE11 isoforms are presented herein from GenBank Accession Nos. NP_{—}005581 (SEQ ID NO: 1), NP_{—}005582 (SEQ ID NO: 2), AAH05241 (SEQ ID NO: 3), and AAC78721 (SEQ ID NO: 4). The amino acid sequences of human RAD50 isoforms are presented herein from GenBank Accession Nos. AAB07119 (SEQ ID NO: 5), NP_{—}005723 (SEQ ID NO: 6), and AAH62603 (SEQ ID NO: 7). The amino acid sequences of human NBS1 isoforms are presented herein from GenBank Accession Nos. BAA28616 (SEQ ID NO: 8), AAC62232 (SEQ ID NO: 9), and AAS59158 (SEQ ID NO: 10).

As used herein, the term “internal control” refers to an amino acid sequence, typically a protein sequence, which does not exhibit reduced levels in a cancer tissue or cell as compared to a non-cancer tissue or cell. Thus, for example, an “internal control” can be used as a “negative control” for assessing whether an MRE11, RAD50, and/or NBS1 protein exhibits reduced levels in a cancer tissue sample or cell without reference to a non-cancer tissue sample or cell.

Suitable proteins that can serve as “internal controls” include, for example and without limitation, β-actin, GAPDH, and cyclophilin. The levels of MRE11, RAD50, and/or NBS1 proteins and internal control protein levels (i.e., non-MRE11, RAD50, and/or NBS1 protein levels) can be determined (e.g., by quantifying the number of MRE11, RAD50, and/or NBS1 proteins), a ratio of MRE11, RAD50, and/or NBS1 protein and non-MRE11, RAD50, and/or NBS1 protein levels can be derived, and the level of MRE11, RAD50, and/or NBS1 proteins within a given a cancer tissue sample or cell can be expressed in terms of the ratio of MRE11, RAD50, and/or NBS1 proteins and non-MRE11, RAD50, and/or NBS1 proteins, wherein a ratio less than a pre-determined threshold ratio indicates reduced MRE11, RAD50, and/or NBS1 protein levels.

In contrast, as used herein, the term “external control” refers to an MRE11, RAD50, and/or NBS1 protein or amino acid sequence from a non-cancer tissue or cell, which MRE11, RAD50, and/or NBS1 protein or amino acid sequence does not exhibit reduced levels in the non-cancer tissue or cell but is being tested for reduced expression in a corresponding cancer tissue or cell. Thus, for example, an “external control” can be used as a “negative control” for assessing whether the MRE11, RAD50, and/or NBS1 protein exhibits reduced levels in a cancer tissue sample or cell by comparing the level of expression (e.g., the number of proteins) in a cancer tissue sample or cell to a corresponding non-cancer tissue sample, such as a tissue sample from a normal donor, or non-cancer cell.

Reduced MRE11, RAD50, and/or NBS1 protein levels can also be assessed on the basis of the percentage or fraction Of cancer cells relative to the total number of cells in a given tissue sample from a cancer patient. By this methodology, for example, the number of MRE11, RAD50, and/or NBS1 proteins in a cancer tissue sample can be quantified and multiplied by the inverse percentage or fraction of cells in the cancer tissue sample. The resulting MRE11, RAD50, and/or NBS1 protein numbers can then be assessed relative to a threshold protein level and, based upon that assessment, the responsiveness of a cancer patient to a therapeutic regimen comprising the administration of a cytotoxic agent can be predicted. More specifically, by this methodology, a protein level for MRE11, RAD50, and/or NBS1 that is less than a threshold level would be predictive of the therapeutic efficacy of such a cytotoxic agent.

Measurement of reduced MRE11, RAD50, and/or NBS1 protein level can, for example, be accomplished by (1) quantifying an MRE11, RAD50, and/or NBS1 protein level in a tissue sample from a cancer patient; (2) quantifying the level of the MRE11, RAD50, and/or NBS1 protein in a tissue sample from a healthy control donor; and (3) comparing the level of the MRE11, RAD50, and/or NBS1 protein in the tissue sample from the cancer patient with the level of the MRE11, RAD50, and/or NBS1 protein in the tissue sample from the control donor. It will be understood that a reduced level of MRE11, RAD50, and/or NBS1 protein in the cancer patient tissue sample as compared to MRE11, RAD50, and/or NBS1 protein in the control donor tissue sample indicates the susceptibility of the cancer patient to treatment with a cytotoxic agent as described herein.

Alternatively, reduced MRE11, RAD50, and/or NBS1 protein levels can be tested by (1) quantifying MRE11, RAD50, and/or NBS1 protein levels in a tissue sample from a cancer patient; (2) quantifying the level of a non-MRE11, RAD50, and/or NBS1 protein in the cancer patient tissue sample, such as, for example, GAPDH or actin; and (3) comparing the level of the MRE11, RAD50, and/or NBS1 protein in the tissue sample from the cancer patient with the level of the non-MRE11, RAD50, and/or NBS1 protein in the cancer patient tissue sample. It will be understood that an reduced level of the MRE11, RAD50, and/or NBS1 protein in the cancer patient tissue sample as compared to the non-MRE11, RAD50, and/or NBS1 protein in the cancer patient tissue sample indicates the susceptibility of the cancer patient to treatment with a cytotoxic agent as described herein.

Within certain aspects of these methods an MRE11, RAD50, and/or NBS1 protein level can be quantified by binding protein in a tissue sample, whether a cancer patient tissue sample or cell, a non-cancer tissue sample or cell from a cancer patient, or a tissue sample or cell from a non-cancer control donor, with an antibody that is specific for MRE11, RAD50, and/or NBS1 protein (see Table 3). Likewise, a non-MRE11, RAD50, and/or NBS1 protein can be quantified by binding protein in a tissue sample, whether a cancer patient tissue sample or cell, a non-cancer tissue sample or cell from a cancer patient, or a tissue sample or cell from a non-cancer control donor, with an antibody that is specific for a non-MRE11, RAD50, and/or NBS1 protein.

Examples of amino acid sequences for MRE11, RAD50, and/or NBS1 protein are presented in Table 3, as are the corresponding accession numbers and sequence identifiers.

In order to identify a patient tissue sample or cell that has reduced protein levels, protein can be isolated from a cancer patient tissue sample or cell and from a non-cancer control tissue sample or cell, the level of a given protein can be determined, and an assessment of reduced protein level can be made by comparing the protein levels determined for a cancer patient tissue sample or cell and a non-cancer control tissue sample or cell.

Alternatively, a cancer patient tissue sample or cell that has reduced MRE11, RAD50, and/or NBS1 protein levels can be identified by isolating protein from a cancer patient tissue sample or cell, determining the levels of an MRE11, RAD50, and/or NBS1 protein and a control protein, and assessing reduced protein levels by comparing the MRE11, RAD50, and/or NBS1 protein and control protein levels within the cancer tissue sample or cell to determine the ratio of protein, wherein an reduced ratio of MRE11, RAD50, and/or NBS1 protein level relative to control protein level indicates a reduced level of MRE11, RAD50, and/or NBS1 protein. As used in this context, a control protein refers to a protein that does not exhibit a reduced level in a cancer tissue or cell. Suitable control proteins include, for example, β-actin, GAPDH, and cyclophilin.

Suitable cancer tissue samples include, for example, blood, lymph node, bone marrow, and/or tumor biopsy samples, including breast or colon tumor biopsy samples, from a cancer patient. Suitable non-cancer control tissue samples include, for example, blood, lymph node, and/or bone marrow samples from a non-cancer donor, such as a healthy, disease-free donor. It will be understood that, regardless of the precise nature or source of the donor tissue sample or cell, it is essential that the donor tissue or cell is known not to exhibit reduced MRE11, RAD50, and/or NBS1 protein levels. Regardless of its source or identity, it will be understood that a suitable non-cancer control tissue sample or cell will be characterized by not exhibiting reduced levels of MRE11, RAD50, and/or NBS1 protein.

Methodologies for quantifying protein levels that can be readily adapted to detecting reduced MRE11, RAD50, and/or NBS1 protein levels are now described in further detail.

1. Production of Anti-MRE11, RAD50, and/or NBS1 Antibodies

Antibodies that are useful in the methodology of the present disclosure can be prepared by using standard techniques. To prepare polyclonal antibodies or “antisera,” an animal is inoculated with an antigen, i.e., a purified immunogenic MRE11, RAD50, or NBS1 protein of the MRN complex, or a peptide thereof, and immunoglobulins are recovered from a fluid, such as blood serum, that contains the immunoglobulins, after the animal has had an immune response. For inoculation, the antigen is preferably bound to a carrier peptide and emulsified using a biologically suitable emulsifying agent, such as Freund's incomplete adjuvant. A variety of mammalian or avian host organisms may be used to prepare polyclonal antibodies against MRE11, RAD50, or NBS1 protein of the MRN complex, or a peptide thereof.

Following immunization, immunoglobulin (Ig) can be purified from the immunized bird or mammal, e.g., goat, rabbit, mouse, rat, or donkey and the like. For certain applications, particularly certain pharmaceutical applications, it is preferable to obtain a composition in which the antibodies are essentially free of antibodies that do not react with the immunogen. This composition is composed virtually entirely of the high titer, monospecific, purified polyclonal antibodies to the immunogen.

Antibodies can be purified by affinity chromatography, using purified MRE11, RAD50, or NBS1 protein of the MRN complex, or a peptide thereof. Purification of antibodies by affinity chromatography is generally known to those skilled in the art (see, for example, U.S. Pat. No. 4,533,630). Briefly, the purified antibody is contacted with the purified immunogen bound to a solid support for a sufficient time and under appropriate conditions for the antibody to bind to the immunogen. Such time and conditions are readily determinable by those skilled in the art. The unbound, unreacted antibody is then removed, such as by washing. The bound antibody is then recovered from the column by eluting the antibodies, so as to yield purified, monospecific polyclonal antibodies.

Monoclonal antibodies can be also prepared, using known hybridoma cell culture techniques. In general, this method involves preparing an antibody-producing fused cell line, e.g., of primary spleen cells fused with a compatible continuous line of myeloma cells, and growing the fused cells either in mass culture or in an animal species, such as a murine species, from which the myeloma cell line used was derived or is compatible. Such antibodies offer many advantages in comparison to those produced by inoculation of animals, as they are highly specific and sensitive and relatively “pure” immunochemically. Immunologically active fragments of the present antibodies are also within the scope of the present disclosure, e.g., the F(ab) fragment and scFv antibodies, as are partially humanized monoclonal antibodies.

Thus, it will be understood by those skilled in the art that the hybridomas herein referred to may be subject to genetic mutation or other changes while still retaining the ability to produce monoclonal antibody of the same desired specificity. The present disclosure encompasses mutants, other derivatives and descendants of the hybridomas.

It will be further understood by those skilled in the art that a monoclonal antibody may be subjected to the techniques of recombinant DNA technology to produce other derivative antibodies, humanized or chimeric molecules or antibody fragments which retain the specificity of the original monoclonal antibody. Such techniques may involve combining DNA encoding the immunoglobulin variable region, or the complementarity determining regions (CDRs), of the monoclonal antibody with DNA coding the constant regions, or constant regions plus framework regions, of a different immunoglobulin, for example, to convert a mouse-derived monoclonal antibody into one having largely human immunoglobulin characteristics (see EP 184187A, 2188638A, herein incorporated by reference).

A biological sample, e.g., a physiological sample which comprises cells may be obtained from a mammal, e.g., a mouse or a human. The cells are lysed to yield an extract which comprises cellular proteins. Alternatively, intact cells, e.g., a tissue sample such as paraffin embedded and/or frozen sections of biopsies, are permeabilized in a manner which permits macromolecules, i.e., antibodies, to enter the cell. The antibodies are then incubated with cells, including permeabilized cells, e.g., prior to flow cytometry, nuclei or the protein extract, e.g., in a western blot, so as to form a complex. The presence, amount and location of the complex is then determined or detected.

The antibodies of the present disclosure may also be coupled to an insoluble or soluble substrate. Soluble substrates include proteins such as bovine serum albumin. Preferably, the antibodies are bound to an insoluble substrate, i.e., a solid support. The antibodies are bound to the support in an amount and manner that allows the antibodies to bind the polypeptide (ligand). The amount of the antibodies used relative to a given substrate depends upon the particular antibody being used, the particular substrate, and the binding efficiency of the antibody to the ligand. The antibodies may be bound to the substrate in any suitable manner. Covalent, noncovalent, or ionic binding may be used. Covalent bonding can be accomplished by attaching the antibodies to reactive groups on the substrate directly or through a linking moiety.

The solid support may be any insoluble material to which the antibodies can be bound and which may be conveniently used in an assay of the present disclosure. Such solid supports include permeable and semipermeable membranes, glass beads, plastic beads, latex beads, plastic micro titer wells or tubes, agarose or dextran particles, sepharose, and diatomaceous earth. Alternatively, the antibodies may be bound to any porous or liquid permeable material, such as a fibrous (paper, felt etc.) strip or sheet, or a screen or net. A binder may be used as long as it does not interfere with the ability of the antibodies to bind the ligands.

2. Immunohistochemical Detection

As used herein, the terms “immunohistochemistry” or “IHC” refer to the detection of an MRE11, RAD50, and/or NBS1 protein in a cell or a tissue section by exploiting the specific binding of an MRE11, RAD50, and/or NBS1 antibody to corresponding protein in a cancer cell or tissue. Immunohistochemical staining is widely used in the diagnosis of abnormal cells such as those found in cancerous tumors and to determine the distribution and localization of biomarkers and differentially expressed proteins in different parts of a biological tissue. Immunohistochemical detection is described in Am J Physiol Regul Integr Comp Physiol. 2011 September; 301(3): R632-R640.

Visualising an antibody-antigen interaction can be accomplished in a number of ways. In the most common instance, an antibody is conjugated to an enzyme, such as peroxidase, that can catalyse a color-producing reaction. Alternatively, the antibody can also be tagged to a fluorophore, such as fluorescein or rhodamine.

Preparation of the sample is critical to maintain cell morphology, tissue architecture and the antigenicity of target epitopes. This requires proper tissue collection, fixation and sectioning. A solution of paraformaldehyde is often used to fixate tissue, but other methods may be used. The tissue may then be sliced or used whole, dependent upon the purpose of the experiment or the tissue itself. Sections can be sliced on a variety of instruments, most commonly a microtome or cryostat and are sliced at a range of 4-40 μm. Before sectioning, the tissue sample may be embedded in a medium, like paraffin wax or cryomedia. Slices may then be mounted on slides for visualizaton through a microscope.

Unlike immunocytochemistry, the tissue does not need to be permeabilized because this has already been accomplished by the microtome blade during sample preparation. Detergents like Triton X-100 are generally used in immunohistochemistry to reduce surface, allowing less reagent to be used to achieve better and more even coverage of the sample.

Depending on the method of fixation and tissue preservation, the sample may require additional steps to make the epitopes available for antibody binding, including de-paraffinization and antigen retrieval (microwave method, enzyme method, hot incubation method). These steps may make the difference between the target antigens staining or not staining.

Depending on the tissue type and the method of antigen detection, endogenous biotin or enzymes may need to be blocked or quenched, respectively, prior to antibody staining. Although antibodies show preferential avidity for specific epitopes, they may partially or weakly bind to sites on nonspecific proteins (also called reactive sites) that are similar to the cognate binding sites on the target antigen. A great amount of non-specific binding causes high background staining which will mask the detection of the target antigen. To reduce background staining in IHC, ICC and other immunostaining methods, samples are incubated with a buffer that blocks the reactive sites to which the primary or secondary antibodies may otherwise bind. Common blocking buffers include normal serum, non-fat dry milk, BSA, or gelatin. Commercial blocking buffers with proprietary formulations are available for greater efficiency.

The antibodies used for specific detection can be polyclonal or monoclonal. Polyclonal antibodies are made by injecting animals with peptide Ag and, after a secondary immune response is stimulated, isolating antibodies from whole serum. Thus, polyclonal antibodies are a heterogeneous mix of antibodies that recognize several epitopes. Monoclonal antibodies show specificity for a single epitope and are therefore considered more specific to the target antigen than polyclonal antibodies.

For IHC detection strategies, antibodies are classified as primary or secondary reagents. Primary antibodies are raised against an antigen of interest and are typically unconjugated (unlabelled), while secondary antibodies are raised against immunoglobulins of the primary antibody species. The secondary antibody is usually conjugated to a linker molecule, such as biotin, that then recruits reporter molecules, or the secondary antibody itself is directly bound to the reporter molecule.

Reporter molecules vary based on the nature of the detection method, the most popular being chromogenic and fluorescence detection mediated by an enzyme or a fluorophore, respectively. With chromogenic reporters, an enzyme label is reacted with a substrate to yield an intensely colored product that can be analyzed with an ordinary light microscope. While the list of enzyme substrates is extensive, alkaline phosphatase (AP) and horseradish peroxidase (HRP) are the two enzymes used most extensively as labels for protein detection. An array of chromogenic, fluorogenic and chemiluminescent substrates is available for use with either enzyme, including DAB or BCIP/NBT, which produce a brown or purple staining, respectively, wherever the enzymes are bound.

Reaction with DAB can be enhanced using nickel, producing a deep purple/black staining. Fluorescent reporters are small, organic molecules used for IHC detection and traditionally include FITC, TRITC, and AMCA, while commercial derivatives, including the Alexa Fluors and Dylight Fluors, show similar enhanced performance but vary in price. For chromogenic and fluorescent detection methods, densitometric analysis of the signal can provide semi- and fully quantitative data, respectively, to correlate the level of reporter signal to the level of protein expression or localization.

The direct method is a one-step staining method and involves a labeled antibody (e.g. FITC-conjugated antiserum reacting directly with the antigen in tissue sections. While this technique utilizes only one antibody and therefore is simple and rapid, the sensitivity is lower due to little signal amplification, in contrast to indirect approaches.

The indirect method involves an unlabeled primary antibody (first layer) that binds to the target antigen in the tissue and a labeled secondary (second layer) that reacts with the primary antibody. As disclosed, herein, the secondary antibody must be raised against the IgG of the animal species in which the primary antibody has been raised. This method is more sensitive than direct detection strategies because of signal amplification due to the binding of several secondary antibodies to each primary antibody if the secondary antibody is conjugated to the fluorescent or enzyme reporter.

Further amplification can be achieved if the secondary antibody is conjugated to several biotin molecules, which can recruit complexes of avidin-streptavidin, or NeutrAvidin proteinbound-enzyme. The difference between these three biotin-binding proteins is their individual binding affinity to endogenous tissue targets leading to nonspecific binding and high background; the ranking of these proteins based on their nonspecific binding affinities, from highest to lowest, is: 1) avidin, 2) streptavidin and 3) Neutravidin protein.

The indirect method, aside from its greater sensitivity, also has the advantage that only a relatively small number of standard conjugated (labeled) secondary antibodies needs to be generated. For example, a labeled secondary antibody raised against rabbit IgG, which can be purchased “off the shelf,” is useful with any primary antibody raised in rabbit. With the direct method, it would be necessary to label each primary antibody for every antigen of interest.

After immunohistochemical staining of the target antigen, a second stain is often applied to provide contrast that helps the primary stain stand out. Many of these stains show specificity for discrete cellular compartments or antigens, while others will stain the whole cell. Both chromogenic and fluorescent dyes are available for IHC to provide a vast array of reagents to fit every experimental design, and include: hematoxylin, Hoechst stain, and DAPI are commonly used.

In immunohistochemical techniques, there are several steps prior to the final staining of the tissue antigen, and many potential problems affect the outcome of the procedure. The major problem areas in IHC staining include strong background staining, weak target antigen staining and autofluorescence. Endogenous biotin or reporter enzymes or primary/secondary antibody cross-reactivity are common causes of strong background staining, while weak staining may be caused by poor enzyme activity or primary antibody potency. Furthermore, autofluorescence may be due to the nature of the tissue or the fixation method. These aspects of IHC tissue prep and antibody staining must be systematically addressed to identify and overcome staining issues.

A variety of molecular pathways are altered in cancer and some of the alterations can be targeted in cancer therapy. Immunohistochemistry can be used to assess which tumors are likely to respond to therapy, by detecting the presence or elevated levels of the molecular target.

3. Immunofluorescent Detection

As used herein, the term “immunofluorescence” refers to a technique used for light microscopy with a fluorescence microscope is used primarily on microbiological samples. This technique uses the specificity of antibodies to their antigen to target fluorescent dyes to specific biomolecule targets within a cell, and therefore allows visualization of the distribution of the target molecule through the sample. Immunofluorescence is a widely used example of immunostaining and is a specific example of immunohistochemistry that makes use of fluorophores to visualize the location of the antibodies. Immunofluorescent detection is described in Am J Physiol Regul Integr Comp Physiol. 301(3): R632-R640 (2011).

Immunofluorescence can be used on tissue sections, cultured cell lines, or individual cells, and may be used to analyze the distribution of proteins, glycans, and small biological and non-biological molecules. Immunofluoresence can be used in combination with other, non-antibody methods of fluorescent staining, for example, use of DAPI to label DNA. Several microscope designs can be used for analysis of immunofluorescence samples; the simplest is the epifluorescence microscope, and the confocal microscope is also widely used. Various super-resolution microscope designs that are capable of much higher resolution can also be used.

There are two classes of immunofluorescence techniques, primary (or direct) and secondary (or indirect). Primary, or direct, immunofluorescence uses a single antibody that is chemically linked to a fluorophore. The antibody recognizes the target molecule and binds to it, and the fluorophore it carries can be detected via microscopy. This technique has several advantages over the secondary (or indirect) protocol below because of the direct conjugation of the antibody to the fluorophore. This reduces the number of steps in the staining procedure making the process faster and can reduce background signal by avoiding some issues with antibody cross-reactivity or non-specificity. However, since the number of fluorescent molecules that can be bound to the primary antibody is limited, direct immunofluorescence is less sensitive than indirect immunofluorescence.

Secondary, or indirect, immunofluorescence uses two antibodies; the unlabeled first (primary) antibody specifically binds the target molecule, and the secondary antibody, which carries the fluorophore, recognises the primary antibody and binds to it. Multiple secondary antibodies can bind a single primary antibody. This provides signal amplification by increasing the number of fluorophore molecules per antigen. This protocol is more complex and time consuming than the primary (or direct) protocol above, but it allows more flexibility because a variety of different secondary antibodies and detection techniques can be used for a given primary antibody.

This protocol is possible because an antibody consists of two parts, a variable region (which recognizes the antigen) and constant region (which makes up the structure of the antibody molecule). It is important to realize that this division is artificial and in reality the antibody molecule is four polypeptide chains: two heavy chains and two light chains. A researcher can generate several primary antibodies that recognize various antigens (have different variable regions), but all share the same constant region. All these antibodies may therefore be recognized by a single secondary antibody. This saves the cost of modifying the primary antibodies to directly carry a fluorophore.

Different primary antibodies with different constant regions are typically generated by raising the antibody in different species. For example, a researcher might create primary antibodies in a goat that recognize several antigens, and then employ dye-coupled rabbit secondary antibodies that recognize the goat antibody constant region (“rabbit anti-goat” antibodies). The researcher may then create a second set of primary antibodies in a mouse that could be recognized by a separate “donkey anti-mouse” secondary antibody. This allows re-use of the difficult-to-make dye-coupled antibodies in multiple experiments.

As with most fluorescence techniques, a significant problem with immunofluorescence is photobleaching. Loss of activity caused by photobleaching can be controlled by reducing the intensity or time-span of light exposure, by increasing the concentration of fluorophores, or by employing more robust fluorophores that are less prone to bleaching (e.g., Alexa Fluors, Seta Fluors, or DyLight Fluors).

Immunofluorescence is only limited to fixed (i.e., dead) cells when structures within the cell are to be visualized because antibodies cannot cross the cell membrane. Proteins in the supernatant or on the outside of the cell membrane can be bound by the antibodies; this allows for living cells to be stained. Depending on the fixative that is being used, proteins of interest might become cross-linked and this could result in either false positive or false negative signals due to non-specific binding.

An alternative approach is using recombinant proteins containing fluorescent protein domains, e.g., green fluorescent protein (GFP). Use of such “tagged” proteins allows determination of their localization in live cells. Even though this seems to be an elegant alternative to immunofluorescence, the cells have to be transfected or transduced with the GFP-tag, and as a consequence they become at least S1 or above organisms that require stricter security standards in a laboratory.

4. Immunoprecipitation

As used herein, the term “immunoprecipitation” refers to a technique for precipitating a protein antigen out of solution using an antibody that specifically binds to that particular protein. This process can be used to isolate and concentrate a particular protein from a sample containing many thousands of different proteins. Immunoprecipitation requires that the antibody be coupled to a solid substrate at some point in the procedure.

Individual protein immunoprecipitation involves using an antibody that is specific for a known protein to isolate that particular protein out of a solution containing many different proteins. These solutions will often be in the form of a crude lysate of a plant or animal tissue. Other sample types could be body fluids or other samples of biological origin.

Immunoprecipitation of intact protein complexes (i.e. antigen along with any proteins or ligands that are bound to it) is known as co-immunoprecipitation (Co-IP). Co-IP works by selecting an antibody that targets a known protein that is believed to be a member of a larger complex of proteins. By targeting this known member with an antibody it may become possible to pull the entire protein complex out of solution and thereby identify unknown members of the complex.

This works when the proteins involved in the complex bind to each other tightly, making it possible to pull multiple members of the complex out of solution by latching onto one member with an antibody. This concept of pulling protein complexes out of solution is sometimes referred to as a “pull-down”. Co-IP is a powerful technique that is used regularly by molecular biologists to analyze protein-protein interactions.

Antibodies that are specific for a particular protein (or group of proteins) can be immobilized on a solid-phase substrate such as super paramagnetic micro beads or on microscopic agarose (non-magnetic) beads. The beads with bound antibodies are then added to the protein mixture, and the proteins that are targeted by the antibodies are captured onto the beads via the antibodies; in other words, they become immunoprecipitated.

Antibodies that are specific for a particular protein, or a group of proteins, are added directly to the mixture of protein. The antibodies have not been attached to a solid-phase support yet. The antibodies are free to float around the protein mixture and bind their targets. As time passes, the beads coated in protein A/G are added to the mixture of antibody and protein. At this point, the antibodies, which are now bound to their targets, will stick to the beads.

5. Western Blotting

The western blot (a/k/a protein immunoblot) is a widely accepted analytical technique used to detect specific proteins in the given sample of tissue homogenate or extract. It uses gel electrophoresis to separate native proteins by 3-D structure or denatured proteins by the length of the polypeptide. The proteins are then transferred to a membrane (typically nitrocellulose or PVDF), where they are stained with antibodies specific to the target protein. There are now many reagent companies that specialize in providing snyinofird (both monoclonal and polyclonal antibodies) against tens of thousands of different proteins.

Samples can be taken from whole tissue or from cell culture. Solid tissues are first broken down mechanically using a bender larger sample volumes), using a homogenizer (smaller volumes), or by sonication. Cells may also be broken open by one of the above mechanical methods. However, virus or environmental samples can be the source of protein and thus western blotting is not restricted to cellular studies only.

Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing and degradation.

A combination of biochemical and mechanical techniques—comprising various types of filtration and centrifugation—can be used to separate different cell compartments and organelles.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to identify a protein

By far the most common type of gel electrophoresis employs polyacrylamide gels and buffers loaded with sodium dodecyl sulfate (SDS). SDS-PAGE (SDS polyacrylamide gel electrophoresis) maintains polypeptides in a denatured state once they have been treated with strong reducing agents to remove secondary and tertiary structure (e.g. disulfide bonds [S—S] to sulfhydryl groups [SH and SH]) and thus allows separation of proteins by their molecular weight. Sampled proteins become covered in the negatively charged SDS and move to the positively charged electrode through the acrylamide mesh of the gel. Smaller proteins migrate faster through this mesh and the proteins are thus separated according to size (usually measured in kilodaltons, kDa). The concentration of acrylamide determines the resolution of the gel—the greater the acrylamide concentration the better the resolution of lower molecular weight proteins. The lower the acrylamide concentration the better the resolution of higher molecular weight proteins. Proteins travel only in one dimension along the gel for most blots.

Samples are loaded into wells in the gel. One lane is usually reserved for a marker or ladder, a commercially available mixture of proteins having defined molecular weights, typically stained so as to form visible, colored bands. When voltage is applied along the gel, proteins migrate through it at different speeds dependent on their size. These different rates of advancement (different electrophoretic mobilities) separate into bands within each lane.

It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection they are moved from within the gel onto a membrane made of nitrocellulose or polyvinylidene difluoride (PVDF). The primary method for transferring the proteins is called electroblotting and uses an electric current to pull proteins from the gel into the PVDF or nitrocellulose membrane. The proteins move from within the gel onto the membrane while maintaining the organization they had within the gel. An older method of transfer involves placing a membrane on top of the gel, and a stack of filter papers on top of that. The entire stack is placed in a buffer solution which moves up the paper by capillary action, bringing the proteins with it. In practice this method is not used as it takes too much time; electroblotting is preferred. As a result of either “blotting” process, the proteins are exposed on a thin surface layer for detection (see below). Both varieties of membrane are chosen for their non-specific protein binding properties (i.e. binds all proteins equally well). Protein binding is based upon hydrophobic interactions, as well as charged interactions between the membrane and protein. Nitrocellulose membranes are cheaper than PVDF, but are far more fragile and do not stand up well to repeated probings.

The uniformity and overall effectiveness of transfer of protein from the gel to the membrane can be checked by staining the membrane with Coomassie Brilliant Blue or Ponceau S dyes. Ponceau S is the more common of the two, due to its higher sensitivity and water solubility, the latter making it easier to subsequently destain and probe the membrane, as described below.

Since the membrane has been chosen for its ability to bind protein and as both antibodies and the target are proteins, steps must be taken to prevent the interactions between the membrane and the antibody used for detection of the target protein. Blocking of non-specific binding is achieved by placing the membrane in a dilute solution of protein—typically 3-5% Bovine serum albumin (BSA) or non-fat dry milk (both are inexpensive) in Tris-Buffered Saline (TBS) or I-Block, with a minute percentage (0.1%) of detergent such as Tween 20 or Triton X-100. The protein in the dilute solution attaches to the membrane in all places where the target proteins have not attached. Thus, when the antibody is added, there is no room on the membrane for it to attach other than on the binding sites of the specific target protein. This reduces “noise” in the final product of the western blot, leading to clearer results, and eliminates false positives.

During the detection process the membrane is “probed” for the protein of interest with a modified antibody which is linked to a reporter enzyme; when exposed to an appropriate substrate this enzyme drives a colorimetric reaction and produces a color. For a variety of reasons, this traditionally takes place in a two-step process, although there are now one-step detection methods available for certain applications.

The primary antibodies are generated when a host species or immune cell culture is exposed to protein of interest (or a part thereof). Normally, this is part of the immune response, whereas here they are harvested and used as sensitive and specific detection tools that bind the protein directly.

After blocking, a dilute solution of primary antibody (generally between 0.5 and 5 micrograms/mL) is incubated with the membrane under gentle agitation. Typically, the solution is composed of buffered saline solution with a small percentage of detergent, and sometimes with powdered milk or BSA. The antibody solution and the membrane can be sealed and incubated together for anywhere from 30 minutes to overnight. It can also be incubated at different temperatures, with higher temperatures being associated with more binding, both specific (to the target protein, the “signal”) and non-specific (“noise”).

After rinsing the membrane to remove unbound primary antibody, the membrane is exposed to another antibody, directed at a species-specific portion of the primary antibody. Antibodies come from animal sources (or animal sourced hybridoma cultures); an anti-mouse secondary will bind to almost any mouse-sourced primary antibody, which allows some cost savings by allowing an entire lab to share a single source of mass-produced antibody, and provides far more consistent results. This is known as a secondary antibody, and due to its targeting properties, tends to be referred to as “anti-mouse,” “anti-goat,” etc. The secondary antibody is usually linked to biotin or to a reporter enzyme such as alkaline phosphatase or horseradish peroxidase. This means that several secondary antibodies will bind to one primary antibody and enhance the signal.

Most commonly, a horseradish peroxidase-linked secondary is used to cleave a chemiluminescent agent, and the reaction product produces luminescence in proportion to the amount of protein. A sensitive sheet of photographic film is placed against the membrane, and exposure to the light from the reaction creates an image of the antibodies bound to the blot. A cheaper but less sensitive approach utilizes a 4-chloronaphthol stain with 1% hydrogen peroxide; reaction of peroxide radicals with 4-chloronaphthol produces a dark purple stain that can be photographed without using specialized photographic film.

As with the ELISPOT and ELISA procedures, the enzyme can be provided with a substrate molecule that will be converted by the enzyme to a colored reaction product that will be visible on the membrane (see the figure below with blue bands).

Another method of secondary antibody detection utilizes a near-infrared (NIR) fluorophore-linked antibody. Light produced from the excitation of a fluorescent dye is static, making fluorescent detection a more precise and accurate measure of the difference in signal produced by labeled antibodies bound to proteins on a western blot. Proteins can be accurately quantified because the signal generated by the different amounts of proteins on the membranes is measured in a static state, as compared to chemiluminescence, in which light is measured in a dynamic state.

A third alternative is to use a radioactive label rather than an enzyme coupled to the secondary antibody, such as labeling an antibody-binding protein like Staphylococcus Protein A or Streptavidin with a radioactive isotope of iodine. Since other methods are safer, quicker, and cheaper, this method is now rarely used; however, an advantage of this approach is the sensitivity of auto-radiography based imaging, which enables highly accurate protein quantification when combined with optical software (e.g. Optiquant).

Methodology for Detecting Mutations, Deletions, and Insertions in Mre11, Rad50, and Nbs1 Genes

Certain mutations within the genes encoding the MRE11, RAD50, and NBS1 proteins that reduce or eliminate one or more function of the MRE11, RAD50, and NBS1 proteins will predictably reduce or eliminate the formation and/or functionality of an MRN complex. It will be understood that mutations, insertions, and/or deletions in one or more of the genes encoding the MRE11, RAD50, and NBS1 proteins, which mutations, insertions, and/or deletions result in one or more amino acid substitutions, amino acid insertions, amino acid deletions, and/or C-terminal truncations within of any one or more of the MRE11, RAD50, and NBS1 proteins, especially, amino acid substitutions, insertions, deletions, and/or C-terminal truncations that eliminate or reduce one or more functions of the MRE11, RAD30, and/or NBS1 proteins, will predictably reduce or eliminate MRN complex formation and/or functionality. An exemplary, non-limiting summary of representative mutations (i.e., amino acid substations), insertions, deletions, and C-terminal truncations within the genes encoding the MRE11, RAD30, and NBS1 proteins is presented in Stracker and Petrini, Nat. Rev. Mol. Cell. Biol. 12:90-103 (2011), see, e.g., Table 1 “Alleles of the MRE11 complex in mice” and the “Supplementary Information” (S1) table presented within the Stracker and Petrini publication.

Thus, analysis of the nucleotide sequences of genes encoding the MRE11, RAD50, and NBS1 proteins and/or the mRNA expressed by those genes can be used diagnostically to determine whether a cell, including a cancer cell, would exhibit reduced MRN complex formation and/or functionality and, as a consequence of that reduced MRN complex formation and/or functionality, whether such a cell would exhibit enhanced sensitivity to a cytotoxic agent as described herein and, moreover, whether a patient afflicted with a cancer associated with reduced MRN complex formation and/or functionality would be susceptible to treatment by the administration of such a cytotoxic agent or a composition comprising one or more cytotoxic agents.

1. MRE11, RAD50, and NBS1 Functional Domains and Activities

The following summarizes representative activities of the MRE11, RAD50, and NBS1 proteins that are essential for the formation and/or functionality of the MRN complex as well as the structural basis for those activities. See, Stracker and Petrini, Nature Rev. Mol. Biol. 12:90-103 (2011). Based upon this description of those activities, one skilled in the art will readily recognize the nature of mutations, insertions, and/or substitutions within the genes encoding the MRE11, RAD50, and NBS1 proteins that will reduce and/or eliminate one or more of those activities.

The MRE11 protein contains the following domains: (1) a nuclease domain, (2) a GAR domain, (3) a DNA binding domain, and (4) an NBS1 binding domain. MRE11 dimerization is critical for DNA binding and is mediated by conserved domains in its N terminus, which include six DNA recognition loops comprising 17-amino acids that form sugar-phosphate contacts in the minor groove of DNA.

MRE11 also possesses a di-Mn-dependent ssDNA endonuclease activity and a 3′-5′ dsDNA exonuclease activity. Paull and Gellert, Mol. Cell 1:969-979 (1998) and Turjillo and Sung, J Biol. Chem. 276:35458-35464 (2001). Thus, the active site of MRE11 is structured to accommodate both ssDNA and dsDNA. The exonuclease function of MRE11 seems to be exerted via melting of the dsDNA terminus, followed by endonucleolytic-type cleavage of the 3′ strand-releasing mononucleotides. Williams et al., (2008), supra; Trujillo et al., J. Biol. Chem. 273:21447-21450 (1998); Paull and Gellert, Mol. Cell 1:969-979 (1998); and Trujillo and Sung, J. Biol. Chem. 276:35458-35464 (2001).

The MRN complex binds to DNA through a globular domain. de Jager et al., Nucl. Acids Res. 30:4425-4431 (2002); de Jager et al. J. Mol. Biol. 339:937-949 (2004); Williams et al., Cell 135:97-109 (2008); Anderson et al., Biol. Chem. 276:37027-37033 (2001); Lee et at, J. Biol. Chem. 278:45171-45181 (2003); and Hobfner et al., Cell 101:789-800 (2000). The DNA binding activity of the MRN complex requires MRE11 and RAD50 and may be influenced by NBS1. Lee (2003), supra; Trujillo et al., J. Biol. Chem. 278:48957-48964 (2003); and Paull and Gellert, Genes Dev. 13:1276-1288 (1999).

The RAD50 protein contains the following functional domains: (1) a hook domain, (2) a coiled-coil domain, (3) a Walker A and B ATPase domain, (4) an MRE11 binding domain, and (5) ATM phosphorylation sites. The RAD50 hook domain functions as a zinc-dependent homodimerization cassette that mediates formation of MRN complex assemblies. This highly-conserved domain is characterized by a central sequence motif of CXXC. Of the RAD50 hook domains analyzed from over 132 species, (95%) have either Pro (85%) or Tyr (10%) at the first X position and 80% have Leu or Val at the second X position, indicating that the residues between the invariant Cys are constrained.

The two Cys residues from one RAD50 protomer coordinate a zinc atom with the two Cys from a second protomer, much like intramolecular coordination of zinc in zinc finger domains. Evans and Hollenberg, Cell 52:1-3 (1988). Zinc-dependent interaction within the hook domains of the two protomers orients their respective coils away from each other at an approximately 140° angle, so that the globular domains of each protomer lie at the distal ends of the assembly. Hobfner et al., Curr. Opin. Struct. Biol. 12:115-122 (2002).

Isosteric mutants of the Cys residues in the CXXC motif have a global effect on MRN complex stability, disrupting the association of RAD50 with MRE11 (Hopfner et al., (2002), supra.), suggesting that the hook domain influences activities at the globular domain, and that the coiled-coil domains, which connect the hook and globular domains, communicate structural perturbations between them. Upon DNA binding by the MRN complex, the RAD50 coiled-coil domains become less flexible and long-range interactions with distal RAD50 protomers become favored. Moreno-Herrero et al., Nature 437:440-443 (2005).

MRE11 and NBS1 associate with RAD50 through RAD50's Walker A and B and extended coiled-coil domains (in which the N-terminal and C-terminal portions of the coils associate in an antiparallel manner). At the apex of the RAD50 coils, where the N-terminal and C-terminal stretches fold back on themselves, is a domain called the RAD50 hook. Stracker et al., DNA Repair (Amst) 3:845-854 (2004); Hopfner et al., Nature 418:562-566 (2002); and Hopfner and Tainer, Curr. Opin. Struct. Boil. 13:249-255 (2003). Rad50 also binds DNA (Raymond and Kleckner, Nucleic Acids Res. 21:3851-3856 (1993)), but the relative contributions of Mre11 and Rad50 to DNA binding at the structural level remain to be established.

The NBS1 protein contains the following functional domains: (1) a Forkhead-associated domain (FHA domain), (2) tandem BRCA1 C-terminal domains (BRCT domains), (3) a CDK phosphorylation site, (4) ATM phosphorylation sites, (5) an MRE11 binding domain, and (6) a C terminal domain. NBS1 regulates the MRN complex by influencing DNA binding and MRE11 nuclease activity. The N-terminal region of NBS1 contains an FHA domain and tandem BRCT domains, which are phosphopeptide-binding modules that participate in diverse phosphorylation-dependent protein interactions. Stacker et al., (2004), supra; Becker et al., Bioinformatics 22:1289-1292 (2006); Xu et al., J. Mol. Biol. 381:361-372 (2008); Williams et al., Cell 139:87-99 (2009); and Lloyd et al., Cell 139:100-111 (2009). FHA and tandem BRCT domains generally function as ‘stand-alone’ phosphopeptide-binding domains. Yu et al., Science 302:639-642 (2003); Manke et al., Science 302:636-639 (2003); and Durocher et al., Mol. Cell 6:1169-1182 (2000).

2. Nucleic Acid Sequencing

Mutations within the genes encoding these and other regions of the MRE11, RAD50, and NBS1 proteins, which result in a reduced MRN complex formation and/or functionality can be readily detected by sequencing the genes encoding MRE11, RAD50, AND NBS1 proteins, mRNA expressed by the Mre11, Rad50, and Nbs1 genes, and/or amplification products (e.g., PCR amplification products) of gene or transcript regions encoding those protein regions.

Chain termination methods were first developed by Frederick Sanger, and can be referred to as Sanger sequencing methods. In chain termination methods, four PCR reactions are performed wherein each reaction is spiked with a single dideoxynucleotide (ddNTP), which is a nucleotide lacking a 3′ hydroxyl group (e.g., ddATP, ddTTP, ddCTP, ddGTP). When a ddNTP is incorporated into a nascent chain of DNA, synthesis of the nascent chain is halted; this generates a mixture of variable length oligonucleotides that can be resolved by size using, for example, DNA electrophoresis in a slab gel or capillary. Any number of detection methods can be used to read the DNA sequence as determined by the relative lengths of oligonucleotides in each of the four reactions, for example, autoradiography, UV light detection, or fluorescent dye detection. Dye termination methods are a variation of chain termination methods whereby each type of ddNTP (e.g., ddATP, ddTTP, ddCTP, ddGTP) is labeled with a different color fluorescent dye. This enables DNA to be sequenced in a single PCR reaction.

Massively Parallel Signature Sequencing (MPSS) is a high-throughput sequencing method that can be used in the methods disclosed herein. It is a bead-based method that utilized adapter ligation followed by adapter decoding to generated hundreds of thousands of short DNA sequences. Further information on this technology can be found in Brenner et al., Nat Biotechnol. 18(6):630-4 (2000); Reinartz et al., Brief Funct Genomic Proteomic. 1(1):95-104 (2002); and U.S. Pat. No. 6,013,445.

Polony sequencing is another high throughput sequencing technology that can be used according to the methods disclosed herein. Polony sequencing combines emulsion PCR, an automated microscope, and ligation-based sequencing chemistry. Further information on this technology can be found in U.S. Patent Publication Nos. 2009/0318298, 2011/0172127, 2010/0047876, and 2009/0099041 and U.S. Pat. No. 7,425,431.

454 pyrosequencing is a high-throughput sequencing method that can be used in the methods disclosed herein. In 454 pyrosequencing, DNA is amplified inside water droplets in an oil solution (emulsion PCR), with each droplet containing a single DNA template attached to a single primer-coated bead, forming a clonal colony. The sequencing machine contains many picolitre-volume wells, each containing a single bead and sequencing enzymes. Luciferase generated light is used to detect individual nucleotides added to the nascent DNA, and the combined data are used to generate sequence read-outs. Further information on this technology can be found in U.S. Pat. Nos. 6,210,891 and 7,648,824.

A high-throughput sequencing method that can be useful in the methods disclosed herein is the sequencing by synthesis (SBS) technology (Illumina®, San Diego, Calif.), which utilizes reversible dye-terminators. Single stranded polynucleotides are first attached to primers on a slide and amplified so that local clonal colonies are formed. Four differentially labeled ddNTPs are added, extending the nascent polynucleotides by one base-pair, after which the non-incorporated nucleotides are washed away. An image of the slide is recorded and the terminal nucleotide for each nascent DNA molecule is determined based upon the color of the fluorescent signal. Then, the dye and the terminal 3′ blocker are chemically removed from the DNA, allowing the next cycle. More information on this technology can be found in U.S. Pat. Nos. 7,985,565; 7,115,400; 7,972,820; and 7,790,418 and U.S. Patent Publication Nos. 2008/0286795, 2002/0055100, and 2007/0015200.

SOLiD (Sequencing by Oligonucleotide Ligation and Detection) sequencing is another high-throughput sequencing method that can be used in the methods disclosed herein. (Applied Biosystems). This method involves multiple rounds of sequencing by ligation, wherein each ligation probe is eight-bases long and each base is effectively probed in two ligation reactions. Base calls are made based upon fluorescence data captured by a camera. More information on this technology can be found in U.S. Patent Publication No. 2009/0181860 and U.S. Pat. No. 7,851,158.

Ion semiconductor sequencing can be a useful high-throughput sequencing technology according to the methods disclosed herein. In ion semiconductor sequencing, the hydrogen ions that are released during polymerization of DNA are detected. A microwell containing a single template DNA strand is flooded with a single polynucleotide, which is incorporated into a nascent strand of DNA if it is complementary to the leading nucleotide of the template strand. The level of hydrogen detected can be used to detect insertion of more than one nucleotide, for example in regions of polynucleotide repeat. Further information on this technology can be found in U.S. Pat. Nos. 7,242,241; 7,888,015; 7,649,358; 7,686,929; and 8,114,591 and U.S. Patent Publication No. 2010/0159461.

DNA nanoball sequencing is another useful high-throughput sequencing technique that can be utilized in the methods disclosed herein. In this technology, rolling circle replication is used to generate DNA nanoballs from DNA fragments. Then, the DNA nanoballs can be anchored into a microarray flow cell, where a process termed unchained sequencing by ligation is used to generate reads about 10 by in length (Complete Genomics). Further information can be found in U.S. Patent Publication Nos. 2009/0011943, 2009/0270273, 2011/0268347, and 2009/0264299.

According to the methods disclosed herein, paired-end tag libraries can be constructed from polynucleotides (e.g., DNA, RNA, mRNA, cDNA, etc.) derived from a tissue sample and used in the high-throughput sequencing technology to increase the speed and/or accuracy sequence assembly. Nucleotides can be sequenced utilizing capture-based technology; alternatively, nucleotides can be sequenced after amplification by PCR. Nucleotides can be treated with bisulfites prior to sequencing in order to identify methylated sequences. Methylation specific PCR can be utilized prior to sequencing in order to determine whether specific loci are methylated. Polynucleotides derived from a leukemia sample can be sequence using paired-end whole exome sequencing (WES), shallow mate-pair whole genome sequencing (sMP-WGS), and/or paired-end RNA sequencing (RNAseq). Polynucleotides derived from a leukemia sample can be sequenced using Illumina® sequencing.

3. Methodology for Assessing Reduced MRN Complex Formation

The association of MRE11, RAD50, and NBS1 into an MRN complex can be determined by a number of methodologies, which are well known in the art including, for example: (1) immunoprecipitation (IP) of intact MRN complexes and (2) cellular localization of MRE11, RAD50, and NBS1 protein subunits and intact MRN complexes by immunofluorescence (IF).

Methodologies for immunoprecipitation of individual MRE11, RAD50, and NBS1 protein subunits as well as the intact MRN complex are described in detail herein as are methodologies for the generation and availability of suitable antibodies for use in those methodologies.

MRN complex formation by the MRE11, RAD50, and NBS1 proteins can be assessed by employing immunofluorescence methodologies to detect the cellular localization of individual proteins as well as the MRN complex before and after exposing a cell to an agent, such as a clastogenic agent, that induces double-strand break (DSB) formation in the cell's chromosomal DNA. Upon DSB formation, the MRE11, RAD50, and NBS1 proteins migrate from the cytoplasm to the sites of damage within the nucleus where they accumulate into discrete foci. Maser et al., Mol. Cell. Biol. 17:6087-6096 (1997) and Nelms et al., Science 280:590-592 (1998).

Methodologies for determining the cytoplasmic and nuclear localization of individual MRE11, RAD50, and NBS1 protein subunits and the intact MRN complex are described in detail in Theunissen and Petrini, Meth. Enzymol. 409:251-284 (2006) (foci formation methodology for assessing the induction of double-strand DNA breaks (DSBs)) and in Petrini and Stracker, Trends in Cell Biol. 13(9):458-462 (2003). General immunofluorescence methodologies are described in detail elsewhere herein.

At early time points (20 minutes to 2 hours) after DSB formation, the coalescence of certain DSB-associating proteins into discrete foci is obscured by the abundant nucleoplasmic pools containing the MRE11, RAD50, and NBS1 protein subunit MRE11, RAD50, and NBS1 proteins. In order to visualize localization of such abundant proteins into early foci, gentle pre-extraction techniques have been developed. Mirzoeva and Petrini, Mol. Cell. Biol. 21:281-288 (2001) and Mirzoeva and Petrini, Mol. Cancer Res. 1:207-218 (2003).

At later time points (4 to 12 hours) after DSB formation, large discrete foci can be detected at the location of those DSBs without pre-extraction even if the protein is abundant. Maser et al., (1997), supra and Williams et al., Curr. Biol. 12:648-653 (2002). The occurrence and number are affected by both the DSB repair proficiency of the cells under study and the dose of clastogenic agent. Although these late foci participate in DSB metabolism, they do not provide insight into recruitment of proteins to DSBs, as most DSBs are repaired within 90 min of induction. Most likely these late foci represent irreparable or slowly repaired lesions. Petrini and Stracker, Trends Cell. Biol. 13:458-462 (2003).

In single labeling experiments, controls are prepared by omitting a primary antibody or replacing it with pre-immune serum. In double labeling experiments, two controls are prepared by omitting one or the other primary antibody and including both secondary antibodies. In double labeling experiments, controls are also prepared for cross-reactivity of each secondary antibody against primary antibodies of different species origin.

Indirect immunofluorescence without pre-extraction can be performed for adherent cells that attach to and grow on glass slides. Cells in suspension require a cytospin or other method of attachment as described in Harlow and Lane, Using Antibodies: A Laboratory Manual (1999).

Adherent cells, such as fibroblasts, can be grown on glass slides or cover glasses. After 24 hours, when the cells are growing logarithmically and reaching subconfluency, they can be treated with a clastogenic agent (or mock treated as a negative control). The cells can then be fixed (e.g., by methanol fixation), permeabilized, and blocked (e.g., with 10% FCS in PBS) prior to immunodetection.

A primary antibody is diluted in ADB and contacted with the fixed cells on the glass slides or cover glasses and incubated for 1 hour at room temperature or overnight at 4° C. After washing with PBS, a secondary antibody is diluted in ADB and contacted with the primary antibody-bound fixed cells.

After incubating, the excess secondary antibody is removed by washing and counterstained with 0.1 mg/ml DAPI (DNA stain) for 1 minute at room temperature. The slides are washed in PBS to remove excess DAPI, fluorescent mounting medium is added, and the slides are viewed under epifluorescence.

Indirect immunofluorescence with pre-extraction (In situ cell fractionation) can be employed for adherent cells (e.g., fibroblasts), which are grown on glass slides. For pre-extraction and fixation, cover glasses are washed with PBS and treated with cytoskeleton buffer (10 mM PIPES, pH 6.8, 100 mM NaCl, 300 mM sucrose, 3 mM MgCl₂, 1 mM EGTA, 0.5% Triton X-100 (v/v)) on ice. Cytoskeleton stripping buffer (10 mM Tris-HCl, pH 7.4, 10 mM NaCl, 3 mM MgCl₂, 1% Tween 40 (v/v), and 0.5% sodium deoxycholate (w/v)) is added and washed with PBS. Cells are fixed in Streck tissue fixative (150 mM 2-bromo-2-nitro-1,3-propanediol (Sigma B0257), 108 mM diazolidinyl urea (Sigma D5146), 10 mM Na Citrate, 50 mM EDTA, pHed to 5.7), washed in PBS, permeabilized in permeabilization solution (100 mM Tris-HCl, pH 7.4, 50 mM EDTA, and 0.5% Triton X-100 (v/v)), washed in PBS, and treated with Alcian Blue to increase adherence of the cells to glass. See, Nickerson et al., Proc. Natl. Acad. Sci. U.S.A. 87:2259-2263 (1990).

Data is analyzed for foci formation with a sample size (typically approximately 75 cells) that is large enough to ensure statistical significance. A P value below 0.05 is generally accepted as an indicator for statistical significance. The P value or significance level is defined as the probability of obtaining the observed result or a more extreme result in the scenario in which both genotypes are equal. If the P value is higher than 0.05 between genotypes A and B, one could argue that the sample sizes were too small to detect significant differences. Before performing an experiment, one should consider the power of the statistical test that will be used. Power is defined as the probability that an experiment of a given sample size will detect statistical significance between two groups. Usually the power of a statistical test should be higher than 90%.

### Kits for Detecting Cancer Cells Exhibiting Reduced MRN Complex Formation or Function

The present disclosure also provides assay kits that can be used in practicing the methods described herein for assessment of the susceptibility of a breast cancer, in particular HNBC or TNBC, as well as a colorectal, urothelial, or other cancer to therapy with one or more of the cytotoxic compounds or compositions disclosed herein. The kit may comprise a first reagent for the detection of expression levels of an Mre11, Rad50, and or Nbs1 gene in the sample of cancerous tissue. The kit may also contain a second reagent for the detection of expression levels of an Mre11, Rad50, and or Nbs1 gene in a sample of a similar but non-cancerous tissue as described herein. In addition, the kit may also include non-specific reagents for performing the test using the reagents.

As used herein the term “kit” is understood to mean a package containing the necessary components necessary to perform the specific evaluations described herein. The components may be individually wrapped or packaged within the “kit”.

The reagent and the test for which it used may be selected to detect expression at the level of mRNA, in which case the reagent is one which provides sequence-specific detection of the mRNA. This may be done using hybridization techniques, in which a sequence specific DNA probe is provided as the reagent and binding of this reagent to the mRNA or a cDNA derived therefrom by amplification is assessed. The specific form of such an assay is not critical, and it may involve detection of binding of labeled reagent (for example with a light emitting or radio label), displacement of a labeled reagent, or competitive binding.

Specific reagents for this purpose can be determined from the cDNA/mRNA sequences disclosed herein that encode each of the individual proteins of the MRN complex. Such reagents are generally 12 to 50 bases length, for example 12 to 30 bases in length, although longer sequences, including full length sequences, can be used to enhance specificity.

The reagent and the test for which it is used may be selected to detect expression at the protein level, for example via immunoassay. Antibodies may be monoclonal or polyclonal antibodies directed against any one of the individual proteins, or against the formed MRN complex. As in the case of hybridization assay, the test format may be binding, competitive binding or displacement. Monoclonal and polyclonal antibodies against MRE11, RAD50, and NBS1 proteins are well known in the art and are described, for example, in Wu et al., SV40 T antigen interacts with Nbs1 to disrupt DNA replication control Genes Dev. 18:1305-1316 (2004); and Wu et al., ATM phosphorylation of Nijmegen breakage syndrome protein is required in a DNA damage response Nature 405:477-482 (2000). Monoclonal and polyclonal antibodies are also commercially available and can be purchased for example from Oncogene (antibodies against MRE11 and NBS1), Upstate (antibodies against RAD50), Cell Signaling Technology (antibodies against MRE11, RAD50, and NBS1), Santa Cruz Biotech (antibodies against MRE11 and RAD50), Antibodies Online (antibodies against MRE11, RAD50, NBS1, and ELISA tests).

A test to assess whether or not a breast cancer cell is hormone negative, or triple negative, can be performed at the same time as, and optionally on the sample in the same reaction, as the test to assess MRN complex or components. In this case, the kit of the present disclosure may also suitably include reagents for assessing hormone responsiveness of the cells in the sample. Such reagents are well known and can be for the same testing modality as the test for MRN complex and individual MRE11, RAD50, and NBS1 proteins, or a different test modality, for example binding to a hormone analog. Immunoassay test kits such as ELISA kits, containing antibodies against estrogen and progesterone receptors as well as HER2/neu are commercially available and sold for example by Antibodies Online, Abcam Plc., and Wilex. FISH test kits are also available for determining presence of these hormone receptors.

TABLE 3
Amino Acid Sequences of MRE11, RAD50, and NBS1 Isoforms

| Sequence Identifier/ | |
|---|---|
| GenBank Accession Number | Description |
| SEQ ID NO: 1 | Met Ser Thr Ala Asp Ala Leu Asp Asp Glu Asn Thr Phe Lys Ile Leu |
| NP_005581 | Val Ala Thr Asp Ile His Leu Gly Phe Met Glu Lys Asp Ala Val Arg |
| | Gly Asn Asp Thr Phe Val Thr Leu Asp Glu Ile Leu Arg Leu Ala Gln |
| | Glu Asn Glu Val Asp Phe Ile Leu Leu Gly Gly Asp Leu Phe His Glu |
| | Asn Lys Pro Ser Arg Lys Thr Leu His Thr Cys Leu Glu Leu Leu Arg |
| | Lys Tyr Cys Met Gly Asp Arg Pro Val Gln Phe Glu Ile Leu Ser Asp |
| | Gln Ser Val Asn Phe Gly Phe Ser Lys Phe Pro Trp Val Asn Tyr Gln |
| | Asp Gly Asn Leu Asn Ile Ser Ile Pro Val Phe Ser Ile His Gly Asn |
| | His Asp Asp Pro Thr Gly Ala Asp Ala Leu Cys Ala Leu Asp Ile Leu |
| | Ser Cys Ala Gly Phe Val Asn His Phe Gly Arg Ser Met Ser Val Glu |
| | Lys Ile Asp Ile Ser Pro Val Leu Leu Gln Lys Gly Ser Thr Lys Ile |
| | Ala Leu Tyr Gly Leu Gly Ser Ile Pro Asp Glu Arg Leu Tyr Arg Met |
| | Phe Val Asn Lys Lys Val Thr Met Leu Arg Pro Lys Glu Asp Glu Asn |
| | Ser Trp Phe Asn Leu Phe Val Ile His Gln Asn Arg Ser Lys His Gly |
| | Ser Thr Asn Phe Ile Pro Glu Gln Phe Leu Asp Asp Phe Ile Asp Leu |
| | Val Ile Trp Gly His Glu His Glu Cys Lys Ile Ala Pro Thr Lys Asn |
| | Glu Gln Gln Leu Phe Tyr Ile Ser Gln Pro Gly Ser Ser Val Val Thr |
| | Ser Leu Ser Pro Gly Glu Ala Val Lys Lys His Val Gly Leu Leu Arg |
| | Ile Lys Gly Arg Lys Met Asn Met His Lys Ile Pro Leu His Thr Val |
| | Arg Gln Phe Phe Met Glu Asp Ile Val Leu Ala Asn His Pro Asp Ile |
| | Phe Asn Pro Asp Asn Pro Lys Val Thr Gln Ala Ile Gln Ser Phe Cys |
| | Leu Glu Lys Ile Glu Glu Met Leu Glu Asn Ala Glu Arg Glu Arg Leu |
| | Gly Asn Ser His Gln Pro Glu Lys Pro Leu Val Arg Leu Arg Val Asp |
| | Tyr Ser Gly Gly Phe Glu Pro Phe Ser Val Leu Arg Phe Ser Gln Lys |
| | Phe Val Asp Arg Val Ala Asn Pro Lys Asp Ile Ile His Phe Phe Arg |
| | His Arg Glu Gln Lys Glu Lys Thr Gly Glu Glu Ile Asn Phe Gly Lys |
| | Leu Ile Thr Lys Pro Ser Glu Gly Thr Thr Leu Arg Val Glu Asp Leu |
| | Val Lys Gln Tyr Phe Gln Thr Ala Glu Lys Asn Val Gln Leu Ser Leu |
| | Leu Thr Glu Arg Gly Met Gly Glu Ala Val Gln Glu Phe Val Asp Lys |
| | Glu Glu Lys Asp Ala Ile Glu Glu Leu Val Lys Tyr Gln Leu Glu Lys |
| | Thr Gln Arg Phe Leu Lys Glu Arg His Ile Asp Ala Leu Glu Asp Lys |
| | Ile Asp Glu Glu Val Arg Arg Phe Arg Glu Thr Arg Gln Lys Asn Thr |
| | Asn Glu Glu Asp Asp Glu Val Arg Glu Ala Met Thr Arg Ala Arg Ala |
| | Leu Arg Ser Gln Ser Glu Glu Ser Ala Ser Ala Phe Ser Ala Asp Asp |
| | Leu Met Ser Ile Asp Leu Ala Glu Gln Met Ala Asn Asp Ser Asp Asp |
| | Ser Ile Ser Ala Ala Thr Asn Lys Gly Arg Gly Arg Gly Arg Gly Arg |
| | Arg Gly Gly Arg Gly Gln Asn Ser Ala Ser Arg Gly Gly Ser Gln Arg |
| | Gly Arg Ala Phe Lys Ser Thr Arg Gln Gln Pro Ser Arg Asn Val Thr |
| | Thr Lys Asn Tyr Ser Glu Val Ile Glu Val Asp Glu Ser Asp Val Glu |
| | Glu Asp Ile Phe Pro Thr Thr Ser Lys Thr Asp Gln Arg Trp Ser Ser |
| | Thr Ser Ser Ser Lys Ile Met Ser Gln Ser Gln Val Ser Lys Gly Val |
| | Asp Phe Glu Ser Ser Glu Asp Asp Asp Asp Asp Pro Phe Met Asn Thr |
| | Ser Ser Leu Arg Arg Asn Arg Arg |
| SEQ ID NO: 2 | Met Ser Thr Ala Asp Ala Leu Asp Asp Glu Asn Thr Phe Lys Ile Leu |
| NP_005582 | Val Ala Thr Asp Ile His Leu Gly Phe Met Glu Lys Asp Ala Val Arg |
| | Gly Asn Asp Thr Phe Val Thr Leu Asp Glu Ile Leu Arg Leu Ala Gln |
| | Glu Asn Glu Val Asp Phe Ile Leu Leu Gly Gly Asp Leu Phe His Glu |
| | Asn Lys Pro Ser Arg Lys Thr Leu His Thr Cys Leu Glu Leu Leu Arg |
| | Lys Tyr Cys Met Gly Asp Arg Pro Val Gln Phe Glu Ile Leu Ser Asp |
| | Gln Ser Val Asn Phe Gly Phe Ser Lys Phe Pro Trp Val Asn Tyr Gln |
| | Asp Gly Asn Leu Asn Ile Ser Ile Pro Val Phe Ser Ile His Gly Asn |
| | His Asp Asp Pro Thr Gly Ala Asp Ala Leu Cys Ala Leu Asp Ile Leu |
| | Ser Cys Ala Gly Phe Val Asn His Phe Gly Arg Ser Met Ser Val Glu |
| | Lys Ile Asp Ile Ser Pro Val Leu Leu Gln Lys Gly Ser Thr Lys Ile |
| | Ala Leu Tyr Gly Leu Gly Ser Ile Pro Asp Glu Arg Leu Tyr Arg Met |
| | Phe Val Asn Lys Lys Val Thr Met Leu Arg Pro Lys Glu Asp Glu Asn |
| | Ser Trp Phe Asn Leu Phe Val Ile His Gln Asn Arg Ser Lys His Gly |
| | Ser Thr Asn Phe Ile Pro Glu Gln Phe Leu Asp Asp Phe Ile Asp Leu |
| | Val Ile Trp Gly His Glu His Glu Cys Lys Ile Ala Pro Thr Lys Asn |
| | Glu Gln Gln Leu Phe Tyr Ile Ser Gln Pro Gly Ser Ser Val Val Thr |
| | Ser Leu Ser Pro Gly Glu Ala Val Lys Lys His Val Gly Leu Leu Arg |
| | Ile Lys Gly Arg Lys Met Asn Met His Lys Ile Pro Leu His Thr Val |
| | Arg Gln Phe Phe Met Glu Asp Ile Val Leu Ala Asn His Pro Asp Ile |
| | Phe Asn Pro Asp Asn Pro Lys Val Thr Gln Ala Ile Gln Ser Phe Cys |
| | Leu Glu Lys Ile Glu Glu Met Leu Glu Asn Ala Glu Arg Glu Arg Leu |
| | Gly Asn Ser His Gln Pro Glu Lys Pro Leu Val Arg Leu Arg Val Asp |
| | Tyr Ser Gly Gly Phe Glu Pro Phe Ser Val Leu Arg Phe Ser Gln Lys |
| | Phe Val Asp Arg Val Ala Asn Pro Lys Asp Ile Ile His Phe Phe Arg |
| | His Arg Glu Gln Lys Glu Lys Thr Gly Glu Glu Ile Asn Phe Gly Lys |
| | Leu Ile Thr Lys Pro Ser Glu Gly Thr Thr Leu Arg Val Glu Asp Leu |
| | Val Lys Gln Tyr Phe Gln Thr Ala Glu Lys Asn Val Gln Leu Ser Leu |
| | Leu Thr Glu Arg Gly Met Gly Glu Ala Val Gln Glu Phe Val Asp Lys |
| | Glu Glu Lys Asp Ala Ile Glu Glu Leu Val Lys Tyr Gln Leu Glu Lys |
| | Thr Gln Arg Phe Leu Lys Glu Arg His Ile Asp Ala Leu Glu Asp Lys |
| | Ile Asp Glu Glu Val Arg Arg Phe Arg Glu Thr Arg Gln Lys Asn Thr |
| | Asn Glu Glu Asp Asp Glu Val Arg Glu Ala Met Thr Arg Ala Arg Ala |
| | Leu Arg Ser Gln Ser Glu Glu Ser Ala Ser Ala Phe Ser Ala Asp Asp |
| | Leu Met Ser Ile Asp Leu Ala Glu Gln Met Ala Asn Asp Ser Asp Asp |
| | Ser Ile Ser Ala Ala Thr Asn Lys Gly Arg Gly Arg Gly Arg Gly Arg |
| | Arg Gly Gly Arg Gly Gln Asn Ser Ala Ser Arg Gly Gly Ser Gln Arg |
| | Gly Arg Ala Asp Thr Gly Leu Glu Thr Ser Thr Arg Ser Arg Asn Ser |
| | Lys Thr Ala Val Ser Ala Ser Arg Asn Met Ser Ile Ile Asp Ala Phe |
| | Lys Ser Thr Arg Gln Gln Pro Ser Arg Asn Val Thr Thr Lys Asn Tyr |
| | Ser Glu Val Ile Glu Val Asp Glu Ser Asp Val Glu Glu Asp Ile Phe |
| | Pro Thr Thr Ser Lys Thr Asp Gln Arg Trp Ser Ser Thr Ser Ser Ser |
| | Lys Ile Met Ser Gln Ser Gln Val Ser Lys Gly Val Asp Phe Glu Ser |
| | Ser Glu Asp Asp Asp Asp Asp Pro Phe Met Asn Thr Ser Ser Leu Arg |
| | Arg Asn Arg Arg |
| SEQ ID NO: 3 | Met Ser Thr Ala Asp Ala Leu Asp Asp Glu Asn Thr Phe Lys Ile Leu |
| AAH05241 | Val Ala Thr Asp Ile His Leu Gly Phe Met Glu Lys Asp Ala Val Arg |
| | Gly Asn Asp Thr Phe Val Thr Leu Asp Glu Ile Leu Arg Leu Ala Gln |
| | Glu Asn Glu Val Asp Phe Ile Leu Leu Gly Gly Asp Leu Phe His Glu |
| | Asn Lys Pro Ser Arg Lys Thr Leu His Thr Cys Leu Glu Leu Leu Arg |
| | Lys Tyr Cys Met Gly Asp Arg Pro Val Gln Phe Glu Ile Leu Ser Asp |
| | Gln Ser Val Asn Phe Gly Phe Ser Lys Phe Pro Trp Val Asn Tyr Gln |
| | Asp Gly Asn Leu Asn Ile Ser Ile Pro Val Phe Ser Ile His Gly Asn |
| | His Asp Asp Pro Thr Gly Ala Asp Ala Leu Cys Ala Leu Asp Ile Leu |
| | Ser Cys Ala Gly Phe Val Asn His Phe Gly Arg Ser Met Ser Val Glu |
| | Lys Ile Asp Ile Ser Pro Val Leu Leu Gln Lys Gly Ser Thr Lys Ile |
| | Ala Leu Tyr Gly Leu Gly Ser Ile Pro Asp Glu Arg Leu Tyr Arg Met |
| | Phe Val Asn Lys Lys Val Thr Met Leu Arg Pro Lys Glu Asp |
| SEQ ID NO: 4 | Met Ser Thr Ala Asp Ala Leu Asp Asp Glu Asn Thr Phe Lys Ile Leu |
| AAC78721 | Val Ala Thr Asp Ile His Leu Gly Phe Met Glu Lys Asp Ala Ala Arg |
| | Gly Asn Asp Thr Phe Val Thr Leu Asp Glu Ile Leu Arg Leu Ala Gln |
| | Glu Asn Glu Val Asp Phe Ile Leu Leu Gly Gly Asp Leu Phe His Glu |
| | Asn Lys Pro Ser Arg Lys Thr Leu His Thr Cys Leu Glu Leu Leu Arg |
| | Lys Tyr Cys Met Gly Asp Arg Pro Val Gln Phe Glu Ile Leu Ser Asp |
| | Gln Ser Val Asn Phe Gly Phe Ser Lys Phe Pro Trp Val Asn Tyr Gln |
| | Asp Gly Asn Leu Asn Ile Ser Ile Pro Val Phe Ser Ile His Gly Asn |
| | His Asp Asp Pro Thr Gly Ala Asp Ala Leu Cys Ala Leu Asp Ile Leu |
| | Ser Cys Ala Gly Phe Val Asn His Phe Gly Arg Ser Met Ser Val Glu |
| | Lys Ile Asp Ile Ser Pro Val Leu Leu Gln Lys Gly Ser Thr Lys Ile |
| | Ala Leu Tyr Gly Leu Gly Ser Ile Pro Asp Glu Arg Leu Tyr Arg Met |
| | Phe Val Asn Lys Lys Val Thr Met Leu Arg Pro Lys Glu Asp Glu Asn |
| | Ser Trp Phe Asn Leu Phe Val Ile His Gln Asn Arg Ser Lys His Gly |
| | Ser Thr Asn Phe Ile Pro Glu Gln Phe Leu Asp Asp Phe Ile Asp Leu |
| | Val Ile Trp Gly His Glu His Glu Cys Lys Ile Ala Pro Thr Lys Asn |
| | Glu Gln Gln Leu Phe Tyr Ile Ser Gln Pro Gly Ser Ser Val Val Thr |
| | Ser Leu Ser Pro Gly Glu Ala Val Lys Lys His Val Gly Leu Leu Arg |
| | Ile Lys Gly Arg Lys Met Asn Met His Lys Ile Pro Leu His Thr Val |
| | Arg Gln Phe Phe Met Glu Asp Ile Val Leu Ala Asn His Pro Asp Ile |
| | Phe Asn Pro Asp Asn Pro Lys Val Thr Gln Ala Ile Gln Ser Phe Cys |
| | Leu Glu Lys Ile Glu Glu Met Leu Glu Asn Ala Glu Arg Glu Arg Leu |
| | Gly Asn Ser His Gln Pro Glu Lys Pro Leu Val Arg Leu Arg Val Asp |
| | Tyr Ser Gly Gly Phe Glu Pro Phe Ser Val Leu Arg Phe Ser Gln Lys |
| | Phe Val Asp Arg Val Ala Asn Pro Lys Asp Ile Ile His Phe Phe Arg |
| | His Arg Glu Gln Lys Glu Lys Thr Gly Glu Glu Ile Asn Phe Gly Lys |
| | Leu Ile Thr Lys Pro Ser Glu Gly Thr Thr Leu Arg Val Glu Asp Leu |
| | Val Lys Gln Tyr Phe Gln Thr Ala Glu Lys Asn Val Gln Leu Ser Leu |
| | Leu Thr Glu Arg Gly Met Gly Glu Ala Val Gln Glu Phe Val Asp Lys |
| | Glu Glu Lys Asp Ala Ile Glu Glu Leu Val Lys Tyr Gln Leu Glu Lys |
| | Thr Gln Arg Phe Leu Lys Glu Arg His Ile Asp Ala Leu Glu Asp Lys |
| | Ile Asp Glu Glu Val Arg Arg Phe Arg Glu Thr Arg Gln Lys Asn Thr |
| | Asn Glu Glu Asp Asp Glu Val Arg Glu Ala Met Thr Arg Ala Arg Ala |
| | Leu Arg Ser Gln Ser Glu Glu Ser Ala Ser Ala Phe Ser Ala Asp Asp |
| | Leu Met Ser Ile Asp Leu Ala Glu Gln Met Ala Asn Asp Ser Asp Asp |
| | Ser Ile Ser Ala Ala Thr Asn Lys Gly Arg Gly Arg Gly Arg Gly Arg |
| | Arg Gly Gly Arg Gly Gln Asn Ser Ala Ser Arg Gly Gly Ser Gln Arg |
| | Gly Arg Ala Phe Lys Ser Thr Arg Gln Gln Pro Ser Arg Asn Val Thr |
| | Thr Lys Asn Tyr Ser Glu Val Ile Glu Val Asp Glu Ser Asp Val Glu |
| | Glu Asp Ile Phe Pro Thr Thr Ser Lys Thr Asp Gln Arg Trp Ser Ser |
| | Thr Ser Ser Ser Lys Ile Met Ser Gln Ser Gln Val Ser Lys Gly Val |
| | Asp Phe Glu Ser Ser Glu Asp Asp Asp Asp Asp Pro Phe Met Asn Thr |
| | Ser Ser Leu Arg Arg Asn Arg Arg |
| SEQ ID NO: 5 | Met Ser Arg Ile Glu Lys Met Ser Ile Leu Gly Val Arg Ser Phe Gly |
| AAB07119 | Ile Glu Asp Lys Asp Lys Gln Ile Ile Thr Phe Phe Ser Pro Leu Thr |
| | Ile Leu Val Gly Pro Asn Gly Ala Gly Lys Thr Thr Ile Ile Glu Cys |
| | Leu Lys Tyr Ile Cys Thr Gly Asp Phe Pro Pro Gly Thr Lys Gly Asn |
| | Thr Phe Val His Asp Pro Lys Val Ala Gln Glu Thr Asp Val Arg Ala |
| | Gln Ile Arg Leu Gln Phe Arg Asp Val Asn Gly Glu Leu Ile Ala Val |
| | Gln Arg Ser Met Val Cys Thr Gln Lys Ser Lys Lys Thr Glu Phe Lys |
| | Thr Leu Glu Gly Val Ile Thr Arg Thr Lys His Gly Glu Lys Val Ser |
| | Leu Ser Ser Lys Cys Ala Glu Ile Asp Arg Glu Met Ile Ser Ser Leu |
| | Gly Val Ser Lys Ala Val Leu Asn Asn Val Ile Phe Cys His Gln Glu |
| | Asp Ser Asn Trp Pro Leu Ser Glu Gly Lys Ala Leu Lys Gln Lys Phe |
| | Asp Glu Ile Phe Ser Ala Thr Arg Tyr Ile Lys Ala Leu Glu Thr Leu |
| | Arg Gln Val Arg Gln Thr Gln Gly Gln Lys Val Lys Glu Tyr Gln Met |
| | Glu Leu Lys Tyr Leu Lys Gln Tyr Lys Glu Lys Ala Cys Glu Ile Arg |
| | Asp Gln Ile Thr Ser Lys Glu Ala Gln Leu Thr Ser Ser Lys Glu Ile |
| | Val Lys Ser Tyr Glu Asn Glu Leu Asp Pro Leu Lys Asn Arg Leu Lys |
| | Glu Ile Glu His Asn Leu Ser Lys Ile Met Lys Leu Asp Asn Glu Ile |
| | Lys Ala Leu Asp Ser Arg Lys Lys Gln Met Glu Lys Asp Asn Ser Glu |
| | Leu Glu Glu Lys Met Glu Lys Val Phe Gln Gly Thr Asp Glu Gln Leu |
| | Asn Asp Leu Tyr His Asn His Gln Arg Thr Val Arg Glu Lys Glu Arg |
| | Lys Leu Val Asp Cys His Arg Glu Leu Glu Lys Leu Asn Lys Glu Ser |
| | Arg Leu Leu Asn Gln Glu Lys Ser Glu Leu Leu Val Glu Gln Gly Arg |
| | Leu Gln Leu Gln Ala Asp Arg His Gln Glu His Ile Arg Ala Arg Asp |
| | Ser Leu Ile Gln Ser Leu Ala Thr Gln Leu Glu Leu Asp Gly Phe Glu |
| | Arg Gly Pro Phe Ser Glu Arg Gln Ile Lys Asn Phe His Lys Leu Val |
| | Arg Glu Arg Gln Glu Gly Glu Ala Lys Thr Ala Asn Gln Leu Met Asn |
| | Asp Phe Ala Glu Lys Glu Thr Leu Lys Gln Lys Gln Ile Asp Glu Ile |
| | Arg Asp Lys Lys Thr Gly Leu Gly Arg Ile Ile Glu Leu Lys Ser Glu |
| | Ile Leu Ser Lys Lys Gln Asn Glu Leu Lys Asn Val Lys Tyr Glu Leu |
| | Gln Gln Leu Glu Gly Ser Ser Asp Arg Ile Leu Glu Leu Asp Gln Glu |
| | Leu Ile Lys Ala Glu Arg Glu Leu Ser Lys Ala Glu Lys Asn Ser Asn |
| | Val Glu Thr Leu Lys Met Glu Val Ile Ser Leu Gln Asn Glu Lys Ala |
| | Asp Leu Asp Arg Thr Leu Arg Lys Leu Asp Gln Glu Met Glu Gln Leu |
| | Asn His His Thr Thr Thr Arg Thr Gln Met Glu Met Leu Thr Lys Asp |
| | Lys Ala Asp Lys Asp Glu Gln Ile Arg Lys Ile Lys Ser Arg His Ser |
| | Asp Glu Leu Thr Ser Leu Leu Gly Tyr Phe Pro Asn Lys Lys Gln Leu |
| | Glu Asp Trp Leu His Ser Lys Ser Lys Glu Ile Asn Gln Thr Arg Asp |
| | Arg Leu Ala Lys Leu Asn Lys Glu Leu Ala Ser Ser Glu Gln Asn Lys |
| SEQ ID NO: 6 | Met Ser Arg Ile Glu Lys Met Ser Ile Leu Gly Val Arg Ser Phe Gly |
| NP_005723 | Ile Glu Asp Lys Asp Lys Gln Ile Ile Thr Phe Phe Ser Pro Leu Thr |
| | Ile Leu Val Gly Pro Asn Gly Ala Gly Lys Thr Thr Ile Ile Glu Cys |
| | Leu Lys Tyr Ile Cys Thr Gly Asp Phe Pro Pro Gly Thr Lys Gly Asn |
| | Thr Phe Val His Asp Pro Lys Val Ala Gln Glu Thr Asp Val Arg Ala |
| | Gln Ile Arg Leu Gln Phe Arg Asp Val Asn Gly Glu Leu Ile Ala Val |
| | Gln Arg Ser Met Val Cys Thr Gln Lys Ser Lys Lys Thr Glu Phe Lys |
| | Thr Leu Glu Gly Val Ile Thr Arg Thr Lys His Gly Glu Lys Val Ser |
| | Leu Ser Ser Lys Cys Ala Glu Ile Asp Arg Glu Met Ile Ser Ser Leu |
| | Gly Val Ser Lys Ala Val Leu Asn Asn Val Ile Phe Cys His Gln Glu |
| | Asp Ser Asn Trp Pro Leu Ser Glu Gly Lys Ala Leu Lys Gln Lys Phe |
| | Asp Glu Ile Phe Ser Ala Thr Arg Tyr Ile Lys Ala Leu Glu Thr Leu |
| | Arg Gln Val Arg Gln Thr Gln Gly Gln Lys Val Lys Glu Tyr Gln Met |
| | Glu Leu Lys Tyr Leu Lys Gln Tyr Lys Glu Lys Ala Cys Glu Ile Arg |
| | Asp Gln Ile Thr Ser Lys Glu Ala Gln Leu Thr Ser Ser Lys Glu Ile |
| | Val Lys Ser Tyr Glu Asn Glu Leu Asp Pro Leu Lys Asn Arg Leu Lys |
| | Glu Ile Glu His Asn Leu Ser Lys Ile Met Lys Leu Asp Asn Glu Ile |
| | Lys Ala Leu Asp Ser Arg Lys Lys Gln Met Glu Lys Asp Asn Ser Glu |
| | Leu Glu Glu Lys Met Glu Lys Val Phe Gln Gly Thr Asp Glu Gln Leu |
| | Asn Asp Leu Tyr His Asn His Gln Arg Thr Val Arg Glu Lys Glu Arg |
| | Lys Leu Val Asp Cys His Arg Glu Leu Glu Lys Leu Asn Lys Glu Ser |
| | Arg Leu Leu Asn Gln Glu Lys Ser Glu Leu Leu Val Glu Gln Gly Arg |
| | Leu Gln Leu Gln Ala Asp Arg His Gln Glu His Ile Arg Ala Arg Asp |
| | Ser Leu Ile Gln Ser Leu Ala Thr Gln Leu Glu Leu Asp Gly Phe Glu |
| | Arg Gly Pro Phe Ser Glu Arg Gln Ile Lys Asn Phe His Lys Leu Val |
| | Arg Glu Arg Gln Glu Gly Glu Ala Lys Thr Ala Asn Gln Leu Met Asn |
| | Asp Phe Ala Glu Lys Glu Thr Leu Lys Gln Lys Gln Ile Asp Glu Ile |
| | Arg Asp Lys Lys Thr Gly Leu Gly Arg Ile Ile Glu Leu Lys Ser Glu |
| | Ile Leu Ser Lys Lys Gln Asn Glu Leu Lys Asn Val Lys Tyr Glu Leu |
| | Gln Gln Leu Glu Gly Ser Ser Asp Arg Ile Leu Glu Leu Asp Gln Glu |
| | Leu Ile Lys Ala Glu Arg Glu Leu Ser Lys Ala Glu Lys Asn Ser Asn |
| | Val Glu Thr Leu Lys Met Glu Val Ile Ser Leu Gln Asn Glu Lys Ala |
| | Asp Leu Asp Arg Thr Leu Arg Lys Leu Asp Gln Glu Met Glu Gln Leu |
| | Asn His His Thr Thr Thr Arg Thr Gln Met Glu Met Leu Thr Lys Asp |
| | Lys Ala Asp Lys Asp Glu Gln Ile Arg Lys Ile Lys Ser Arg His Ser |
| | Asp Glu Leu Thr Ser Leu Leu Gly Tyr Phe Pro Asn Lys Lys Gln Leu |
| | Glu Asp Trp Leu His Ser Lys Ser Lys Glu Ile Asn Gln Thr Arg Asp |
| | Arg Leu Ala Lys Leu Asn Lys Glu Leu Ala Ser Ser Glu Gln Asn Lys |
| SEQ ID NO: 7 | Met Ser Arg Ile Glu Lys Met Ser Ile Leu Gly Val Arg Ser Phe Gly |
| AAH62603 | Ile Glu Asp Lys Asp Lys Gln Ile Ile Thr Phe Phe Ser Pro Leu Thr |
| | Ile Leu Val Gly Pro Asn Gly Ala Gly Lys Thr Thr Ile Ile Glu Cys |
| | Leu Lys Tyr Ile Cys Thr Gly Asp Phe Pro Pro Gly Thr Lys Gly Asn |
| | Thr Phe Val His Asp Pro Lys Val Ala Gln Glu Thr Asp Val Arg Ala |
| | Gln Ile Arg Leu Gln Phe Arg Asp Val Asn Gly Glu Leu Ile Ala Val |
| | Gln Arg Ser Met Val Cys Thr Gln Lys Ser Lys Lys Thr Glu Phe Lys |
| | Thr Leu Glu Gly Val Ile Thr Arg Thr Lys His Gly Glu Lys Val Ser |
| | Leu Ser Ser Lys Cys Ala Glu Ile Asp Arg Glu Met Ile Ser Ser Leu |
| | Gly Val Ser Lys Ala Val Leu Asn Asn Val Ile Phe Cys His Gln Glu |
| | Asp Ser Asn Trp Pro Leu Ser Glu Gly Lys Ala Leu Lys Gln Lys Phe |
| | Asp Glu Ile Phe Ser Ala Thr Arg Tyr Ile Lys Ala Leu Glu Thr Leu |
| | Arg Gln Val Arg Gln Thr Gln Gly Gln Lys Val Lys Glu Tyr Gln Met |
| | Glu Leu Lys Tyr Leu Lys Gln Tyr Lys Glu Lys Ala Cys Glu Ile Arg |
| | Asp Gln Ile Thr Ser Lys Glu Ala Gln Leu Thr Ser Ser Lys Glu Ile |
| | Val Lys Ser Tyr Glu Asn Glu Leu Asp Pro Leu Lys Asn Arg Leu Lys |
| | Glu Ile Glu His Asn Leu Ser Lys Ile Met Lys Leu Asp Asn Glu Ile |
| | Lys Ala Leu Asp Ser Arg Lys Lys Gln Met Glu Lys Asp Asn Ser Glu |
| | Leu Glu Glu Lys Met Glu Lys Val Phe Gln Gly Thr Asp Glu Gln Leu |
| | Asn Asp Leu Tyr His Asn His Gln Arg Thr Val Arg Glu Lys Glu Arg |
| | Lys Leu Val Asp Cys His Arg Glu Leu Glu Lys Leu Asn Lys Glu Ser |
| | Arg Leu Leu Asn Gln Glu Lys Ser Glu Leu Leu Val Glu Gln Gly Arg |
| | Leu Gln Leu Gln Ala Asp Arg His Gln Glu His Ile Arg Ala Arg Asp |
| | Ser Leu Ile Gln Ser Leu Ala Thr Gln Leu Glu Leu Asp Gly Phe Glu |
| | Arg Gly Pro Phe Ser Glu Arg Gln Ile Lys Asn Phe His Lys Leu Val |
| | Arg Glu Arg Gln Glu Gly Glu Ala Lys Thr Ala Asn Gln Leu Met Asn |
| | Asp Phe Ala Glu Lys Glu Thr Leu Lys Gln Lys Gln Ile Asp Glu Ile |
| | Arg Asp Lys Lys Thr Gly Leu Gly Arg Ile Ile Glu Leu Lys Ser Glu |
| | Ile Leu Ser Lys Lys Gln Asn Glu Leu Lys Asn Val Lys Tyr Glu Leu |
| | Gln Gln Leu Glu Gly Ser Ser Asp Arg Ile Leu Glu Leu Asp Gln Glu |
| | Leu Ile Lys Ala Glu Arg Glu Leu Ser Lys Ala Glu Lys Asn Ser Asn |
| | Val Glu Thr Leu Lys Met Glu Val Ile Ser Leu Gln Asn Glu Lys Ala |
| | Asp Leu Asp Arg Thr Leu Arg Lys Leu Asp Gln Glu Met Glu Gln Leu |
| | Asn His His Thr Thr Thr Arg Thr Gln Met Glu Met Leu Thr Lys Asp |
| | Lys Ala Asp Lys Asp Glu Gln Ile Arg Lys Lys Lys Lys |
| SEQ ID NO: 8 | Met Trp Lys Leu Leu Pro Ala Ala Gly Pro Ala Gly Gly Glu Pro Tyr |
| BAA28616 | Arg Leu Leu Thr Gly Val Glu Tyr Val Val Gly Arg Lys Asn Cys Ala |
| | Ile Leu Ile Glu Asn Asp Gln Ser Ile Ser Arg Asn His Ala Val Leu |
| | Thr Ala Asn Phe Ser Val Thr Asn Leu Ser Gln Thr Asp Glu Ile Pro |
| | Val Leu Thr Leu Lys Asp Asn Ser Lys Tyr Gly Thr Phe Val Asn Glu |
| | Glu Lys Met Gln Asn Gly Phe Ser Arg Thr Leu Lys Ser Gly Asp Gly |
| | Ile Thr Phe Gly Val Phe Gly Ser Lys Phe Arg Ile Glu Tyr Glu Pro |
| | Leu Val Ala Cys Ser Ser Cys Leu Asp Val Ser Gly Lys Thr Ala Leu |
| | Asn Gln Ala Ile Leu Gln Leu Gly Gly Phe Thr Val Asn Asn Trp Thr |
| | Glu Glu Cys Thr His Leu Val Met Val Ser Val Lys Val Thr Ile Lys |
| | Thr Ile Cys Ala Leu Ile Cys Gly Arg Pro Ile Val Lys Pro Glu Tyr |
| | Phe Thr Glu Phe Leu Lys Ala Val Glu Ser Lys Lys Gln Pro Pro Gln |
| | Ile Glu Ser Phe Tyr Pro Pro Leu Asp Glu Pro Ser Ile Gly Ser Lys |
| | Asn Val Asp Leu Ser Gly Arg Gln Glu Arg Lys Gln Ile Phe Lys Gly |
| | Lys Thr Phe Ile Phe Leu Asn Ala Lys Gln His Lys Lys Leu Ser Ser |
| | Ala Val Val Phe Gly Gly Gly Glu Ala Arg Leu Ile Thr Glu Glu Asn |
| | Glu Glu Glu His Asn Phe Phe Leu Ala Pro Gly Thr Cys Val Val Asp |
| | Thr Gly Ile Thr Asn Ser Gln Thr Leu Ile Pro Asp Cys Gln Lys Lys |
| | Trp Ile Gln Ser Ile Met Asp Met Leu Gln Arg Gln Gly Leu Arg Pro |
| | Ile Pro Glu Ala Glu Ile Gly Leu Ala Val Ile Phe Met Thr Thr Lys |
| | Asn Tyr Cys Asp Pro Gln Gly His Pro Ser Thr Gly Leu Lys Thr Thr |
| | Thr Pro Gly Pro Ser Leu Ser Gln Gly Val Ser Val Asp Glu Lys Leu |
| | Met Pro Ser Ala Pro Val Asn Thr Thr Thr Tyr Val Ala Asp Thr Glu |
| | Ser Glu Gln Ala Asp Thr Trp Asp Leu Ser Glu Arg Pro Lys Glu Ile |
| | Lys Val Ser Lys Met Glu Gln Lys Phe Arg Met Leu Ser Gln Asp Ala |
| | Pro Thr Val Lys Glu Ser Cys Lys Thr Ser Ser Asn Asn Asn Ser Met |
| | Val Ser Asn Thr Leu Ala Lys Met Arg Ile Pro Asn Tyr Gln Leu Ser |
| | Pro Thr Lys Leu Pro Ser Ile Asn Lys Ser Lys Asp Arg Ala Ser Gln |
| | Gln Gln Gln Thr Asn Ser Ile Arg Asn Tyr Phe Gln Pro Ser Thr Lys |
| | Lys Arg Glu Arg Asp Glu Glu Asn Gln Glu Met Ser Ser Cys Lys Ser |
| | Ala Arg Ile Glu Thr Ser Cys Ser Leu Leu Glu Gln Thr Gln Pro Ala |
| | Thr Pro Ser Leu Trp Lys Asn Lys Glu Gln His Leu Ser Glu Asn Glu |
| | Pro Val Asp Thr Asn Ser Asp Asn Asn Leu Phe Thr Asp Thr Asp Leu |
| | Lys Ser Ile Val Lys Asn Ser Ala Ser Lys Ser His Ala Ala Glu Lys |
| | Leu Arg Ser Asn Lys Lys Arg Glu Met Asp Asp Val Ala Ile Glu Asp |
| | Glu Val Leu Glu Gln Leu Phe Lys Asp Thr Lys Pro Glu Leu Glu Ile |
| | Asp Val Lys Val Gln Lys Gln Glu Glu Asp Val Asn Val Arg Lys Arg |
| | Pro Arg Met Asp Ile Glu Thr Asn Asp Thr Phe Ser Asp Glu Ala Val |
| SEQ ID NO: 9 | Met Trp Lys Leu Leu Pro Ala Ala Gly Pro Ala Gly Gly Glu Pro Tyr |
| AAC62232 | Arg Leu Leu Thr Gly Val Glu Tyr Val Val Gly Arg Lys Asn Cys Ala |
| | Ile Leu Ile Glu Asn Asp Gln Ser Ile Ser Arg Asn His Ala Val Leu |
| | Thr Ala Asn Phe Ser Val Thr Asn Leu Ser Gln Thr Asp Glu Ile Pro |
| | Val Leu Thr Leu Lys Asp Asn Ser Lys Tyr Gly Thr Phe Val Asn Glu |
| | Glu Lys Met Gln Asn Gly Phe Ser Arg Thr Leu Lys Ser Gly Asp Gly |
| | Ile Thr Phe Gly Val Phe Gly Ser Lys Phe Arg Ile Glu Tyr Glu Pro |
| | Leu Val Ala Cys Ser Ser Cys Leu Asp Val Ser Gly Lys Thr Ala Leu |
| | Asn Gln Ala Ile Leu Gln Leu Gly Gly Phe Thr Val Asn Asn Trp Thr |
| | Glu Glu Cys Thr His Leu Val Met Val Ser Val Lys Val Thr Ile Lys |
| | Thr Ile Cys Ala Leu Ile Cys Gly Arg Pro Ile Val Lys Pro Glu Tyr |
| | Phe Thr Glu Phe Leu Lys Ala Val Glu Ser Lys Lys Gln Pro Pro Gln |
| | Ile Glu Ser Phe Tyr Pro Pro Leu Asp Glu Pro Ser Ile Gly Ser Lys |
| | Asn Val Asp Leu Ser Gly Arg Gln Glu Arg Lys Gln Ile Phe Lys Gly |
| | Lys Thr Phe Ile Phe Leu Asn Ala Lys Gln His Lys Lys Leu Ser Ser |
| | Ala Val Val Phe Gly Gly Gly Glu Ala Arg Leu Ile Thr Glu Glu Asn |
| | Glu Glu Glu His Asn Phe Phe Leu Ala Pro Gly Thr Cys Val Val Asp |
| | Thr Gly Ile Thr Asn Ser Gln Thr Leu Ile Pro Asp Cys Gln Lys Lys |
| | Trp Ile Gln Ser Ile Met Asp Met Leu Gln Arg Gln Gly Leu Arg Pro |
| | Ile Pro Glu Ala Glu Ile Gly Leu Ala Val Ile Phe Met Thr Thr Lys |
| | Asn Tyr Cys Asp Pro Gln Gly His Pro Ser Thr Gly Leu Lys Thr Thr |
| | Thr Pro Gly Pro Ser Leu Ser Gln Gly Val Ser Val Asp Glu Lys Leu |
| | Met Pro Ser Ala Pro Val Asn Thr Thr Thr Tyr Val Ala Asp Thr Glu |
| | Ser Glu Gln Ala Asp Thr Trp Asp Leu Ser Glu Arg Pro Lys Glu Ile |
| | Lys Val Ser Lys Met Glu Gln Lys Phe Arg Met Leu Ser Gln Asp Ala |
| | Pro Thr Val Lys Glu Ser Cys Lys Thr Ser Ser Asn Asn Asn Ser Met |
| | Val Ser Asn Thr Leu Ala Lys Met Arg Ile Pro Asn Tyr Gln Leu Ser |
| | Pro Thr Lys Leu Pro Ser Ile Asn Lys Ser Lys Asp Arg Ala Ser Gln |
| | Gln Gln Gln Thr Asn Ser Ile Arg Asn Tyr Phe Gln Pro Ser Thr Lys |
| | Lys Arg Glu Arg Asp Glu Glu Asn Gln Glu Met Ser Ser Cys Lys Ser |
| | Ala Arg Ile Glu Thr Ser Cys Ser Leu Leu Glu Gln Thr Gln Pro Ala |
| | Thr Pro Ser Leu Trp Lys Asn Lys Glu Gln His Leu Ser Glu Asn Glu |
| | Pro Val Asp Thr Asn Ser Asp Asn Asn Leu Phe Thr Asp Thr Asp Leu |
| | Lys Ser Ile Val Lys Asn Ser Ala Ser Lys Ser His Ala Ala Glu Lys |
| | Leu Arg Ser Asn Lys Lys Arg Glu Met Asp Asp Val Ala Ile Glu Asp |
| | Glu Val Leu Glu Gln Leu Phe Lys Asp Thr Lys Pro Glu Leu Glu Ile |
| | Asp Val Lys Val Gln Lys Gln Glu Glu Asp Val Asn Val Arg Lys Arg |
| | Pro Arg Met Asp Ile Glu Thr Asn Asp Thr Phe Ser Asp Glu Ala Val |
| SEQ ID NO: 10 | Met Trp Lys Leu Leu Pro Ala Ala Gly Pro Ala Gly Gly Glu Pro Tyr |
| AAS59158 | Arg Leu Leu Thr Gly Val Glu Tyr Val Val Gly Arg Lys Asn Cys Ala |
| | Ile Leu Ile Glu Asn Asp Gln Ser Ile Ser Arg Asn His Ala Val Leu |
| | Thr Ala Asn Phe Ser Val Thr Asn Leu Ser Gln Thr Asp Glu Ile Pro |
| | Val Leu Thr Leu Lys Asp Asn Ser Lys Tyr Gly Thr Phe Val Asn Glu |
| | Glu Lys Met Gln Asn Gly Phe Ser Arg Thr Leu Lys Ser Gly Asp Gly |
| | Ile Thr Phe Gly Val Phe Gly Ser Lys Phe Arg Ile Glu Tyr Glu Pro |
| | Leu Val Ala Cys Ser Ser Cys Leu Asp Val Ser Gly Lys Thr Ala Leu |
| | Asn Gln Ala Ile Leu Gln Leu Gly Gly Phe Thr Val Asn Asn Trp Thr |
| | Glu Glu Cys Thr His Leu Val Met Val Ser Val Lys Val Thr Ile Lys |
| | Thr Ile Cys Ala Leu Ile Cys Gly Arg Pro Ile Val Lys Pro Glu Tyr |
| | Phe Thr Glu Phe Leu Lys Ala Val Glu Ser Lys Lys Gln Pro Pro Gln |
| | Ile Glu Ser Phe Tyr Pro Pro Leu Asp Glu Pro Ser Ile Gly Ser Lys |
| | Asn Val Asp Leu Ser Gly Arg Gln Glu Arg Lys Gln Ile Phe Lys Gly |
| | Lys Thr Phe Ile Phe Leu Asn Ala Lys Gln His Lys Lys Leu Ser Ser |
| | Ala Val Val Phe Gly Gly Gly Glu Ala Arg Leu Ile Thr Glu Glu Asn |
| | Glu Glu Glu His Asn Phe Phe Leu Ala Pro Gly Thr Cys Val Val Asp |
| | Thr Gly Ile Thr Asn Ser Gln Thr Leu Ile Pro Asp Cys Gln Lys Lys |
| | Trp Ile Gln Ser Ile Met Asp Met Leu Gln Arg Gln Gly Leu Arg Pro |
| | Ile Pro Glu Ala Glu Ile Gly Leu Ala Val Ile Phe Met Thr Thr Lys |
| | Asn Tyr Cys Asp Pro Gln Gly His Pro Ser Thr Gly Leu Lys Thr Thr |
| | Thr Pro Gly Pro Ser Leu Ser Gln Gly Val Ser Val Asp Glu Lys Leu |
| | Met Pro Ser Ala Pro Val Asn Thr Thr Thr Tyr Val Ala Asp Thr Glu |
| | Ser Glu Gln Ala Asp Thr Trp Asp Leu Ser Glu Arg Pro Lys Glu Ile |
| | Lys Val Ser Lys Met Glu Gln Lys Phe Arg Met Leu Ser Gln Asp Ala |
| | Pro Thr Val Lys Glu Ser Cys Lys Thr Ser Ser Asn Asn Asn Ser Met |
| | Val Ser Asn Thr Leu Ala Lys Met Arg Ile Pro Asn Tyr Gln Leu Ser |
| | Pro Thr Lys Leu Pro Ser Ile Asn Lys Ser Lys Asp Arg Ala Ser Gln |
| | Gln Gln Gln Thr Asn Ser Ile Arg Asn Tyr Phe Gln Pro Ser Thr Lys |
| | Lys Arg Glu Arg Asp Glu Glu Asn Gln Glu Met Ser Ser Cys Lys Ser |
| | Ala Arg Ile Glu Thr Ser Cys Ser Leu Leu Glu Gln Thr Gln Pro Ala |
| | Thr Pro Ser Leu Trp Lys Asn Lys Glu Gln His Leu Ser Glu Asn Glu |
| | Pro Val Asp Thr Asn Ser Asp Asn Asn Leu Phe Thr Asp Thr Asp Leu |
| | Lys Ser Ile Val Lys Asn Ser Ala Ser Lys Ser His Ala Ala Glu Lys |
| | Leu Arg Ser Asn Lys Lys Arg Glu Met Asp Asp Val Ala Ile Glu Asp |
| | Glu Val Leu Glu Gln Leu Phe Lys Asp Thr Lys Pro Glu Leu Glu Ile |
| | Asp Val Lys Val Gln Lys Gln Glu Glu Asp Val Asn Val Arg Lys Arg |
| | Pro Arg Met Asp Ile Glu Thr Asn Asp Thr Phe Ser Asp Glu Ala Val |

TABLE 4
Nucleotide Sequences Encoding MRE11, RAD50, and NBS1 Isoforms

| Sequence Identifier | Description | |
|---|---|---|
| SEQ ID NO: 11 | atgwsnacng cngaygcnyt ngaygaygar aayacnttya arathytngt ngcnacngay | 60 |
| | athcayytng gnttyatgga raargaygcn gtnmgnggna aygayacntt ygtnacnytn | 120 |
| | gaygarathy tnmgnytngc ncargaraay gargtngayt tyathytnyt nggnggngay | 180 |
| | ytnttycayg araayaarcc nwsnmgnaar acnytncaya cntgyytnga rytnytnmgn | 240 |
| | aartaytgya tgggngaymg nccngtncar ttygarathy tnwsngayca rwsngtnaay | 300 |
| | ttyggnttyw snaarttycc ntgggtnaay taycargayg gnaayytnaa yathwsnath | 360 |
| | ccngtnttyw snathcaygg naaycaygay gayccnacng gngcngaygc nytntgygcn | 420 |
| | ytngayathy tnwsntgygc nggnttygtn aaycayttyg gnmgnwsnat gwsngtngar | 480 |
| | aarathgaya thwsnccngt nytnytncar aarggnwsna cnaarathgc nytntayggn | 540 |
| | ytnggnwsna thccngayga rmgnytntay mgnatgttyg tnaayaaraa rgtnacnatg | 600 |
| | ytnmgnccna argargayga raaywsntgg ttyaayytnt tygtnathca ycaraaymgn | 660 |
| | wsnaarcayg gnwsnacnaa yttyathccn garcarttyy tngaygaytt yathgayytn | 720 |
| | gtnathtggg gncaygarca ygartgyaar athgcnccna cnaaraayga rcarcarytn | 780 |
| | ttytayathw sncarccngg nwsnwsngtn gtnacnwsny tnwsnccngg ngargcngtn | 840 |
| | aaraarcayg tnggnytnyt nmgnathaar ggnmgnaara tgaayatgca yaarathccn | 900 |
| | ytncayacng tnmgncartt yttyatggar gayathgtny tngcnaayca yccngayath | 960 |
| | ttyaayccng ayaayccnaa rgtnacncar gcnathcarw snttytgyyt ngaraarath | 1020 |
| | gargaratgy tngaraaygc ngarmgngar mgnytnggna aywsncayca rccngaraar | 1080 |
| | ccnytngtnm gnytnmgngt ngaytaywsn ggnggnttyg arccnttyws ngtnytnmgn | 1140 |
| | ttywsncara arttygtnga ymgngtngcn aayccnaarg ayathathca yttyttymgn | 1200 |
| | caymgngarc araargaraa racnggngar garathaayt tyggnaaryt nathacnaar | 1260 |
| | ccnwsngarg gnacnacnyt nmgngtngar gayytngtna arcartaytt ycaracngcn | 1320 |
| | garaaraayg tncarytnws nytnytnacn garmgnggna tgggngargc ngtncargar | 1380 |
| | ttygtngaya argargaraa rgaygcnath gargarytng tnaartayca rytngaraar | 1440 |
| | acncarmgnt tyytnaarga rmgncayath gaygcnytng argayaarat hgaygargar | 1500 |
| | gtnmgnmgnt tymgngarac nmgncaraar aayacnaayg argargayga ygargtnmgn | 1560 |
| | gargcnatga cnmgngcnmg ngcnytnmgn wsncarwsng argarwsngc nwsngcntty | 1620 |
| | wsngcngayg ayytnatgws nathgayytn gcngarcara tggcnaayga ywsngaygay | 1680 |
| | wsnathwsng cngcnacnaa yaarggnmgn ggnmgnggnm gnggnmgnmg nggnggnmgn | 1740 |
| | ggncaraayw sngcnwsnmg nggnggnwsn carmgnggnm gngcnttyaa rwsnacnmgn | 1800 |
| | carcarccnw snmgnaaygt nacnacnaar aaytaywsng argtnathga rgtngaygar | 1860 |
| | wsngaygtng argargayat httyccnacn acnwsnaara cngaycarmg ntggwsnwsn | 1920 |
| | acnwsnwsnw snaarathat gwsncarwsn cargtnwsna arggngtnga yttygarwsn | 1980 |
| | wsngargayg aygaygayga yccnttyatg aayacnwsnw snytnmgnmg naaymgnmgn | 2040 |
| SEQ ID NO: 12 | atgwsnacng cngaygcnyt ngaygaygar aayacnttya arathytngt ngcnacngay | 60 |
| | athcayytng gnttyatgga raargaygcn gtnmgnggna aygayacntt ygtnacnytn | 120 |
| | gaygarathy tnmgnytngc ncargaraay gargtngayt tyathytnyt nggnggngay | 180 |
| | ytnttycayg araayaarcc nwsnmgnaar acnytncaya cntgyytnga rytnytnmgn | 240 |
| | aartaytgya tgggngaymg nccngtncar ttygarathy tnwsngayca rwsngtnaay | 300 |
| | ttyggnttyw snaarttycc ntgggtnaay taycargayg gnaayytnaa yathwsnath | 360 |
| | ccngtnttyw snathcaygg naaycaygay gayccnacng gngcngaygc nytntgygcn | 420 |
| | ytngayathy tnwsntgygc nggnttygtn aaycayttyg gnmgnwsnat gwsngtngar | 480 |
| | aarathgaya thwsnccngt nytnytncar aarggnwsna cnaarathgc nytntayggn | 540 |
| | ytnggnwsna thccngayga rmgnytntay mgnatgttyg tnaayaaraa rgtnacnatg | 600 |
| | ytnmgnccna argargayga raaywsntgg ttyaayytnt tygtnathca ycaraaymgn | 660 |
| | wsnaarcayg gnwsnacnaa yttyathccn garcarttyy tngaygaytt yathgayytn | 720 |
| | gtnathtggg gncaygarca ygartgyaar athgcnccna cnaaraayga rcarcarytn | 780 |
| | ttytayathw sncarccngg nwsnwsngtn gtnacnwsny tnwsnccngg ngargcngtn | 840 |
| | aaraarcayg tnggnytnyt nmgnathaar ggnmgnaara tgaayatgca yaarathccn | 900 |
| | ytncayacng tnmgncartt yttyatggar gayathgtny tngcnaayca yccngayath | 960 |
| | ttyaayccng ayaayccnaa rgtnacncar gcnathcarw snttytgyyt ngaraarath | 1020 |
| | gargaratgy tngaraaygc ngarmgngar mgnytnggna aywsncayca rccngaraar | 1080 |
| | ccnytngtnm gnytnmgngt ngaytaywsn ggnggnttyg arccnttyws ngtnytnmgn | 1140 |
| | ttywsncara arttygtnga ymgngtngcn aayccnaarg ayathathca yttyttymgn | 1200 |
| | caymgngarc araargaraa racnggngar garathaayt tyggnaaryt nathacnaar | 1260 |
| | ccnwsngarg gnacnacnyt nmgngtngar gayytngtna arcartaytt ycaracngcn | 1320 |
| | garaaraayg tncarytnws nytnytnacn garmgnggna tgggngargc ngtncargar | 1380 |
| | ttygtngaya argargaraa rgaygcnath gargarytng tnaartayca rytngaraar | 1440 |
| | acncarmgnt tyytnaarga rmgncayath gaygcnytng argayaarat hgaygargar | 1500 |
| | gtnmgnmgnt tymgngarac nmgncaraar aayacnaayg argargayga ygargtnmgn | 1560 |
| | gargcnatga cnmgngcnmg ngcnytnmgn wsncarwsng argarwsngc nwsngcntty | 1620 |
| | wsngcngayg ayytnatgws nathgayytn gcngarcara tggcnaayga ywsngaygay | 1680 |
| | wsnathwsng cngcnacnaa yaarggnmgn ggnmgnggnm gnggnmgnmg nggnggnmgn | 1740 |
| | ggncaraayw sngcnwsnmg nggnggnwsn carmgnggnm gngcngayac nggnytngar | 1800 |
| | acnwsnacnm gnwsnmgnaa ywsnaaracn gcngtnwsng cnwsnmgnaa yatgwsnath | 1860 |
| | athgaygcnt tyaarwsnac nmgncarcar ccnwsnmgna aygtnacnac naaraaytay | 1920 |
| | wsngargtna thgargtnga ygarwsngay gtngargarg ayathttycc nacnacnwsn | 1980 |
| | aaracngayc armgntggws nwsnacnwsn wsnwsnaara thatgwsnca rwsncargtn | 2040 |
| | wsnaarggng tngayttyga rwsnwsngar gaygaygayg aygayccntt yatgaayacn | 2100 |
| | wsnwsnytnm gnmgnaaymg nmgn | 2124 |
| SEQ ID NO: 13 | atgwsnacng cngaygcnyt ngaygaygar aayacnttya arathytngt ngcnacngay | 60 |
| | athcayytng gnttyatgga raargaygcn gtnmgnggna aygayacntt ygtnacnytn | 120 |
| | gaygarathy tnmgnytngc ncargaraay gargtngayt tyathytnyt nggnggngay | 180 |
| | ytnttycayg araayaarcc nwsnmgnaar acnytncaya cntgyytnga rytnytnmgn | 240 |
| | aartaytgya tgggngaymg nccngtncar ttygarathy tnwsngayca rwsngtnaay | 300 |
| | ttyggnttyw snaarttycc ntgggtnaay taycargayg gnaayytnaa yathwsnath | 360 |
| | ccngtnttyw snathcaygg naaycaygay gayccnacng gngcngaygc nytntgygcn | 420 |
| | ytngayathy tnwsntgygc nggnttygtn aaycayttyg gnmgnwsnat gwsngtngar | 480 |
| | aarathgaya thwsnccngt nytnytncar aarggnwsna cnaarathgc nytntayggn | 540 |
| | ytnggnwsna thccngayga rmgnytntay mgnatgttyg tnaayaaraa rgtnacnatg | 600 |
| | ytnmgnccna argargay | 618 |
| SEQ ID NO: 14 | atgwsnacng cngaygcnyt ngaygaygar aayacnttya arathytngt ngcnacngay | 60 |
| | athcayytng gnttyatgga raargaygcn gcnmgnggna aygayacntt ygtnacnytn | 120 |
| | gaygarathy tnmgnytngc ncargaraay gargtngayt tyathytnyt nggnggngay | 180 |
| | ytnttycayg araayaarcc nwsnmgnaar acnytncaya cntgyytnga rytnytnmgn | 240 |
| | aartaytgya tgggngaymg nccngtncar ttygarathy tnwsngayca rwsngtnaay | 300 |
| | ttyggnttyw snaarttycc ntgggtnaay taycargayg gnaayytnaa yathwsnath | 360 |
| | ccngtnttyw snathcaygg naaycaygay gayccnacng gngcngaygc nytntgygcn | 420 |
| | ytngayathy tnwsntgygc nggnttygtn aaycayttyg gnmgnwsnat gwsngtngar | 480 |
| | aarathgaya thwsnccngt nytnytncar aarggnwsna cnaarathgc nytntayggn | 540 |
| | ytnggnwsna thccngayga rmgnytntay mgnatgttyg tnaayaaraa rgtnacnatg | 600 |
| | ytnmgnccna argargayga raaywsntgg ttyaayytnt tygtnathca ycaraaymgn | 660 |
| | wsnaarcayg gnwsnacnaa yttyathccn garcarttyy tngaygaytt yathgayytn | 720 |
| | gtnathtggg gncaygarca ygartgyaar athgcnccna cnaaraayga rcarcarytn | 780 |
| | ttytayathw sncarccngg nwsnwsngtn gtnacnwsny tnwsnccngg ngargcngtn | 840 |
| | aaraarcayg tnggnytnyt nmgnathaar ggnmgnaara tgaayatgca yaarathccn | 900 |
| | ytncayacng tnmgncartt yttyatggar gayathgtny tngcnaayca yccngayath | 960 |
| | ttyaayccng ayaayccnaa rgtnacncar gcnathcarw snttytgyyt ngaraarath | 1020 |
| | gargaratgy tngaraaygc ngarmgngar mgnytnggna aywsncayca rccngaraar | 1080 |
| | ccnytngtnm gnytnmgngt ngaytaywsn ggnggnttyg arccnttyws ngtnytnmgn | 1140 |
| | ttywsncara arttygtnga ymgngtngcn aayccnaarg ayathathca yttyttymgn | 1200 |
| | caymgngarc araargaraa racnggngar garathaayt tyggnaaryt nathacnaar | 1260 |
| | ccnwsngarg gnacnacnyt nmgngtngar gayytngtna arcartaytt ycaracngcn | 1320 |
| | garaaraayg tncarytnws nytnytnacn garmgnggna tgggngargc ngtncargar | 1380 |
| | ttygtngaya argargaraa rgaygcnath gargarytng tnaartayca rytngaraar | 1440 |
| | acncarmgnt tyytnaarga rmgncayath gaygcnytng argayaarat hgaygargar | 1500 |
| | gtnmgnmgnt tymgngarac nmgncaraar aayacnaayg argargayga ygargtnmgn | 1560 |
| | gargcnatga cnmgngcnmg ngcnytnmgn wsncarwsng argarwsngc nwsngcntty | 1620 |
| | wsngcngayg ayytnatgws nathgayytn gcngarcara tggcnaayga ywsngaygay | 1680 |
| | wsnathwsng cngcnacnaa yaarggnmgn ggnmgnggnm gnggnmgnmg nggnggnmgn | 1740 |
| | ggncaraayw sngcnwsnmg nggnggnwsn carmgnggnm gngcnttyaa rwsnacnmgn | 1800 |
| | carcarccnw snmgnaaygt nacnacnaar aaytaywsng argtnathga rgtngaygar | 1860 |
| | wsngaygtng argargayat httyccnacn acnwsnaara cngaycarmg ntggwsnwsn | 1920 |
| | acnwsnwsnw snaarathat gwsncarwsn cargtnwsna arggngtnga yttygarwsn | 1980 |
| | wsngargayg aygaygayga yccnttyatg aayacnwsnw snytnmgnmg naaymgnmgn | 2040 |
| SEQ ID NO: 15 | atgwsnmgna thgaraarat gwsnathytn ggngtnmgnw snttyggnat hgargayaar | 60 |
| | gayaarcara thathacntt yttywsnccn ytnacnathy tngtnggncc naayggngcn | 120 |
| | ggnaaracna cnathathga rtgyytnaar tayathtgya cnggngaytt yccnccnggn | 180 |
| | acnaarggna ayacnttygt ncaygayccn aargtngcnc argaracnga ygtnmgngcn | 240 |
| | carathmgny tncarttymg ngaygtnaay ggngarytna thgcngtnca rmgnwsnatg | 300 |
| | gtntgyacnc araarwsnaa raaracngar ttyaaracny tngarggngt nathacnmgn | 360 |
| | acnaarcayg gngaraargt nwsnytnwsn wsnaartgyg cngarathga ymgngaratg | 420 |
| | athwsnwsny tnggngtnws naargcngtn ytnaayaayg tnathttytg ycaycargar | 480 |
| | gaywsnaayt ggccnytnws ngarggnaar gcnytnaarc araarttyga ygarathtty | 540 |
| | wsngcnacnm gntayathaa rgcnytngar acnytnmgnc argtnmgnca racncarggn | 600 |
| | caraargtna argartayca ratggarytn aartayytna arcartayaa rgaraargcn | 660 |
| | tgygarathm gngaycarat hacnwsnaar gargcncary tnacnwsnws naargarath | 720 |
| | gtnaarwsnt aygaraayga rytngayccn ytnaaraaym gnytnaarga rathgarcay | 780 |
| | aayytnwsna arathatgaa rytngayaay garathaarg cnytngayws nmgnaaraar | 840 |
| | caratggara argayaayws ngarytngar garaaratgg araargtntt ycarggnacn | 900 |
| | gaygarcary tnaaygayyt ntaycayaay caycarmgna cngtnmgnga raargarmgn | 960 |
| | aarytngtng aytgycaymg ngarytngar aarytnaaya argarwsnmg nytnytnaay | 1020 |
| | cargaraarw sngarytnyt ngtngarcar ggnmgnytnc arytncargc ngaymgncay | 1080 |
| | cargarcaya thmgngcnmg ngaywsnytn athcarwsny tngcnacnca rytngarytn | 1140 |
| | gayggnttyg armgnggncc nttywsngar mgncaratha araayttyca yaarytngtn | 1200 |
| | mgngarmgnc argarggnga rgcnaaracn gcnaaycary tnatgaayga yttygcngar | 1260 |
| | aargaracny tnaarcaraa rcarathgay garathmgng ayaaraarac nggnytnggn | 1320 |
| | mgnathathg arytnaarws ngarathytn wsnaaraarc araaygaryt naaraaygtn | 1380 |
| | aartaygary tncarcaryt ngarggnwsn wsngaymgna thytngaryt ngaycargar | 1440 |
| | ytnathaarg cngarmgnga rytnwsnaar gcngaraara aywsnaaygt ngaracnytn | 1500 |
| | aaratggarg tnathwsnyt ncaraaygar aargcngayy tngaymgnac nytnmgnaar | 1560 |
| | ytngaycarg aratggarca rytnaaycay cayacnacna cnmgnacnca ratggaratg | 1620 |
| | ytnacnaarg ayaargcnga yaargaygar carathmgna arathaarws nmgncaywsn | 1680 |
| | gaygarytna cnwsnytnyt nggntaytty ccnaayaara arcarytnga rgaytggytn | 1740 |
| | caywsnaarw snaargarat haaycaracn mgngaymgny tngcnaaryt naayaargar | 1800 |
| | ytngcnwsnw sngarcaraa yaaraaycay athaayaayg arytnaarmg naargargar | 1860 |
| | carytnwsnw sntaygarga yaarytntty gaygtntgyg gnwsncarga yttygarwsn | 1920 |
| | gayytngaym gnytnaarga rgarathgar aarwsnwsna arcarmgngc natgytngcn | 1980 |
| | ggngcnacng cngtntayws ncarttyath acncarytna cngaygaraa ycarwsntgy | 2040 |
| | tgyccngtnt gycarmgngt nttycaracn gargcngary tncargargt nathwsngay | 2100 |
| | ytncarwsna arytnmgnyt ngcnccngay aarytnaarw snacngarws ngarytnaar | 2160 |
| | aaraargara armgnmgnga ygaratgytn ggnytngtnc cnatgmgnca rwsnathath | 2220 |
| | gayytnaarg araargarat hccngarytn mgnaayaary tncaraaygt naaymgngay | 2280 |
| SEQ ID NO: 16 | atgwsnmgna thgaraarat gwsnathytn ggngtnmgnw snttyggnat hgargayaar | 60 |
| | gayaarcara thathacntt yttywsnccn ytnacnathy tngtnggncc naayggngcn | 120 |
| | ggnaaracna cnathathga rtgyytnaar tayathtgya cnggngaytt yccnccnggn | 180 |
| | acnaarggna ayacnttygt ncaygayccn aargtngcnc argaracnga ygtnmgngcn | 240 |
| | carathmgny tncarttymg ngaygtnaay ggngarytna thgcngtnca rmgnwsnatg | 300 |
| | gtntgyacnc araarwsnaa raaracngar ttyaaracny tngarggngt nathacnmgn | 360 |
| | acnaarcayg gngaraargt nwsnytnwsn wsnaartgyg cngarathga ymgngaratg | 420 |
| | athwsnwsny tnggngtnws naargcngtn ytnaayaayg tnathttytg ycaycargar | 480 |
| | gaywsnaayt ggccnytnws ngarggnaar gcnytnaarc araarttyga ygarathtty | 540 |
| | wsngcnacnm gntayathaa rgcnytngar acnytnmgnc argtnmgnca racncarggn | 600 |
| | caraargtna argartayca ratggarytn aartayytna arcartayaa rgaraargcn | 660 |
| | tgygarathm gngaycarat hacnwsnaar gargcncary tnacnwsnws naargarath | 720 |
| | gtnaarwsnt aygaraayga rytngayccn ytnaaraaym gnytnaarga rathgarcay | 780 |
| | aayytnwsna arathatgaa rytngayaay garathaarg cnytngayws nmgnaaraar | 840 |
| | caratggara argayaayws ngarytngar garaaratgg araargtntt ycarggnacn | 900 |
| | gaygarcary tnaaygayyt ntaycayaay caycarmgna cngtnmgnga raargarmgn | 960 |
| | aarytngtng aytgycaymg ngarytngar aarytnaaya argarwsnmg nytnytnaay | 1020 |
| | cargaraarw sngarytnyt ngtngarcar ggnmgnytnc arytncargc ngaymgncay | 1080 |
| | cargarcaya thmgngcnmg ngaywsnytn athcarwsny tngcnacnca rytngarytn | 1140 |
| | gayggnttyg armgnggncc nttywsngar mgncaratha araayttyca yaarytngtn | 1200 |
| | mgngarmgnc argarggnga rgcnaaracn gcnaaycary tnatgaayga yttygcngar | 1260 |
| | aargaracny tnaarcaraa rcarathgay garathmgng ayaaraarac nggnytnggn | 1320 |
| | mgnathathg arytnaarws ngarathytn wsnaaraarc araaygaryt naaraaygtn | 1380 |
| | aartaygary tncarcaryt ngarggnwsn wsngaymgna thytngaryt ngaycargar | 1440 |
| | ytnathaarg cngarmgnga rytnwsnaar gcngaraara aywsnaaygt ngaracnytn | 1500 |
| | aaratggarg tnathwsnyt ncaraaygar aargcngayy tngaymgnac nytnmgnaar | 1560 |
| | ytngaycarg aratggarca rytnaaycay cayacnacna cnmgnacnca ratggaratg | 1620 |
| | ytnacnaarg ayaargcnga yaargaygar carathmgna arathaarws nmgncaywsn | 1680 |
| | gaygarytna cnwsnytnyt nggntaytty ccnaayaara arcarytnga rgaytggytn | 1740 |
| | caywsnaarw snaargarat haaycaracn mgngaymgny tngcnaaryt naayaargar | 1800 |
| | ytngcnwsnw sngarcaraa yaaraaycay athaayaayg arytnaarmg naargargar | 1860 |
| | carytnwsnw sntaygarga yaarytntty gaygtntgyg gnwsncarga yttygarwsn | 1920 |
| | gayytngaym gnytnaarga rgarathgar aarwsnwsna arcarmgngc natgytngcn | 1980 |
| | ggngcnacng cngtntayws ncarttyath acncarytna cngaygaraa ycarwsntgy | 2040 |
| | tgyccngtnt gycarmgngt nttycaracn gargcngary tncargargt nathwsngay | 2100 |
| | ytncarwsna arytnmgnyt ngcnccngay aarytnaarw snacngarws ngarytnaar | 2160 |
| | aaraargara armgnmgnga ygaratgytn ggnytngtnc cnatgmgnca rwsnathath | 2220 |
| | gayytnaarg araargarat hccngarytn mgnaayaary tncaraaygt naaymgngay | 2280 |
| SEQ ID NO: 17 | atgwsnmgna thgaraarat gwsnathytn ggngtnmgnw snttyggnat hgargayaar | 60 |
| | gayaarcara thathacntt yttywsnccn ytnacnathy tngtnggncc naayggngcn | 120 |
| | ggnaaracna cnathathga rtgyytnaar tayathtgya cnggngaytt yccnccnggn | 180 |
| | acnaarggna ayacnttygt ncaygayccn aargtngcnc argaracnga ygtnmgngcn | 240 |
| | carathmgny tncarttymg ngaygtnaay ggngarytna thgcngtnca rmgnwsnatg | 300 |
| | gtntgyacnc araarwsnaa raaracngar ttyaaracny tngarggngt nathacnmgn | 360 |
| | acnaarcayg gngaraargt nwsnytnwsn wsnaartgyg cngarathga ymgngaratg | 420 |
| | athwsnwsny tnggngtnws naargcngtn ytnaayaayg tnathttytg ycaycargar | 480 |
| | gaywsnaayt ggccnytnws ngarggnaar gcnytnaarc araarttyga ygarathtty | 540 |
| | wsngcnacnm gntayathaa rgcnytngar acnytnmgnc argtnmgnca racncarggn | 600 |
| | caraargtna argartayca ratggarytn aartayytna arcartayaa rgaraargcn | 660 |
| | tgygarathm gngaycarat hacnwsnaar gargcncary tnacnwsnws naargarath | 720 |
| | gtnaarwsnt aygaraayga rytngayccn ytnaaraaym gnytnaarga rathgarcay | 780 |
| | aayytnwsna arathatgaa rytngayaay garathaarg cnytngayws nmgnaaraar | 840 |
| | caratggara argayaayws ngarytngar garaaratgg araargtntt ycarggnacn | 900 |
| | gaygarcary tnaaygayyt ntaycayaay caycarmgna cngtnmgnga raargarmgn | 960 |
| | aarytngtng aytgycaymg ngarytngar aarytnaaya argarwsnmg nytnytnaay | 1020 |
| | cargaraarw sngarytnyt ngtngarcar ggnmgnytnc arytncargc ngaymgncay | 1080 |
| | cargarcaya thmgngcnmg ngaywsnytn athcarwsny tngcnacnca rytngarytn | 1140 |
| | gayggnttyg armgnggncc nttywsngar mgncaratha araayttyca yaarytngtn | 1200 |
| | mgngarmgnc argarggnga rgcnaaracn gcnaaycary tnatgaayga yttygcngar | 1260 |
| | aargaracny tnaarcaraa rcarathgay garathmgng ayaaraarac nggnytnggn | 1320 |
| | mgnathathg arytnaarws ngarathytn wsnaaraarc araaygaryt naaraaygtn | 1380 |
| | aartaygary tncarcaryt ngarggnwsn wsngaymgna thytngaryt ngaycargar | 1440 |
| | ytnathaarg cngarmgnga rytnwsnaar gcngaraara aywsnaaygt ngaracnytn | 1500 |
| | aaratggarg tnathwsnyt ncaraaygar aargcngayy tngaymgnac nytnmgnaar | 1560 |
| | ytngaycarg aratggarca rytnaaycay cayacnacna cnmgnacnca ratggaratg | 1620 |
| | ytnacnaarg ayaargcnga yaargaygar carathmgna araaraaraa r | 1671 |
| SEQ ID NO: 18 | atgtggaary tnytnccngc ngcnggnccn gcnggnggng arccntaymg nytnytnacn | 60 |
| | ggngtngart aygtngtngg nmgnaaraay tgygcnathy tnathgaraa ygaycarwsn | 120 |
| | athwsnmgna aycaygcngt nytnacngcn aayttywsng tnacnaayyt nwsncaracn | 180 |
| | gaygarathc cngtnytnac nytnaargay aaywsnaart ayggnacntt ygtnaaygar | 240 |
| | garaaratgc araayggntt ywsnmgnacn ytnaarwsng gngayggnat hacnttyggn | 300 |
| | gtnttyggnw snaarttymg nathgartay garccnytng tngcntgyws nwsntgyytn | 360 |
| | gaygtnwsng gnaaracngc nytnaaycar gcnathytnc arytnggngg nttyacngtn | 420 |
| | aayaaytgga cngargartg yacncayytn gtnatggtnw sngtnaargt nacnathaar | 480 |
| | acnathtgyg cnytnathtg yggnmgnccn athgtnaarc cngartaytt yacngartty | 540 |
| | ytnaargcng tngarwsnaa raarcarccn ccncarathg arwsnttyta yccnccnytn | 600 |
| | gaygarccnw snathggnws naaraaygtn gayytnwsng gnmgncarga rmgnaarcar | 660 |
| | athttyaarg gnaaracntt yathttyytn aaygcnaarc arcayaaraa rytnwsnwsn | 720 |
| | gcngtngtnt tyggnggngg ngargcnmgn ytnathacng argaraayga rgargarcay | 780 |
| | aayttyttyy tngcnccngg nacntgygtn gtngayacng gnathacnaa ywsncaracn | 840 |
| | ytnathccng aytgycaraa raartggath carwsnatha tggayatgyt ncarmgncar | 900 |
| | ggnytnmgnc cnathccnga rgcngarath ggnytngcng tnathttyat gacnacnaar | 960 |
| | aaytaytgyg ayccncargg ncayccnwsn acnggnytna aracnacnac nccnggnccn | 1020 |
| | wsnytnwsnc arggngtnws ngtngaygar aarytnatgc cnwsngcncc ngtnaayacn | 1080 |
| | acnacntayg tngcngayac ngarwsngar cargcngaya cntgggayyt nwsngarmgn | 1140 |
| | ccnaargara thaargtnws naaratggar caraarttym gnatgytnws ncargaygcn | 1200 |
| | ccnacngtna argarwsntg yaaracnwsn wsnaayaaya aywsnatggt nwsnaayacn | 1260 |
| | ytngcnaara tgmgnathcc naaytaycar ytnwsnccna cnaarytncc nwsnathaay | 1320 |
| | aarwsnaarg aymgngcnws ncarcarcar caracnaayw snathmgnaa ytayttycar | 1380 |
| | ccnwsnacna araarmgnga rmgngaygar garaaycarg aratgwsnws ntgyaarwsn | 1440 |
| | gcnmgnathg aracnwsntg ywsnytnytn garcaracnc arccngcnac nccnwsnytn | 1500 |
| | tggaaraaya argarcarca yytnwsngar aaygarccng tngayacnaa ywsngayaay | 1560 |
| | aayytnttya cngayacnga yytnaarwsn athgtnaara aywsngcnws naarwsncay | 1620 |
| | gcngcngara arytnmgnws naayaaraar mgngaratgg aygaygtngc nathgargay | 1680 |
| | gargtnytng arcarytntt yaargayacn aarccngary tngarathga ygtnaargtn | 1740 |
| | caraarcarg argargaygt naaygtnmgn aarmgnccnm gnatggayat hgaracnaay | 1800 |
| | gayacnttyw sngaygargc ngtnccngar wsnwsnaara thwsncarga raaygarath | 1860 |
| | ggnaaraarm gngarytnaa rgargaywsn ytntggwsng cnaargarat hwsnaayaay | 1920 |
| | gayaarytnc argaygayws ngaratgytn ccnaaraary tnytnytnac ngarttymgn | 1980 |
| | wsnytngtna thaaraayws nacnwsnmgn aayccnwsng gnathaayga ygaytayggn | 2040 |
| | carytnaara ayttyaaraa rttyaaraar gtnacntayc cnggngcngg naarytnccn | 2100 |
| | cayathathg gnggnwsnga yytnathgcn caycaygcnm gnaaraayac ngarytngar | 2160 |
| | gartggytnm gncargarat ggargtncar aaycarcayg cnaargarga rwsnytngcn | 2220 |
| | gaygayytnt tymgntayaa yccntayytn aarmgnmgnm gn | 2262 |
| SEQ ID NO: 19 | atgtggaary tnytnccngc ngcnggnccn gcnggnggng arccntaymg nytnytnacn | 60 |
| | ggngtngart aygtngtngg nmgnaaraay tgygcnathy tnathgaraa ygaycarwsn | 120 |
| | athwsnmgna aycaygcngt nytnacngcn aayttywsng tnacnaayyt nwsncaracn | 180 |
| | gaygarathc cngtnytnac nytnaargay aaywsnaart ayggnacntt ygtnaaygar | 240 |
| | garaaratgc araayggntt ywsnmgnacn ytnaarwsng gngayggnat hacnttyggn | 300 |
| | gtnttyggnw snaarttymg nathgartay garccnytng tngcntgyws nwsntgyytn | 360 |
| | gaygtnwsng gnaaracngc nytnaaycar gcnathytnc arytnggngg nttyacngtn | 420 |
| | aayaaytgga cngargartg yacncayytn gtnatggtnw sngtnaargt nacnathaar | 480 |
| | acnathtgyg cnytnathtg yggnmgnccn athgtnaarc cngartaytt yacngartty | 540 |
| | ytnaargcng tngarwsnaa raarcarccn ccncarathg arwsnttyta yccnccnytn | 600 |
| | gaygarccnw snathggnws naaraaygtn gayytnwsng gnmgncarga rmgnaarcar | 660 |
| | athttyaarg gnaaracntt yathttyytn aaygcnaarc arcayaaraa rytnwsnwsn | 720 |
| | gcngtngtnt tyggnggngg ngargcnmgn ytnathacng argaraayga rgargarcay | 780 |
| | aayttyttyy tngcnccngg nacntgygtn gtngayacng gnathacnaa ywsncaracn | 840 |
| | ytnathccng aytgycaraa raartggath carwsnatha tggayatgyt ncarmgncar | 900 |
| | ggnytnmgnc cnathccnga rgcngarath ggnytngcng tnathttyat gacnacnaar | 960 |
| | aaytaytgyg ayccncargg ncayccnwsn acnggnytna aracnacnac nccnggnccn | 1020 |
| | wsnytnwsnc arggngtnws ngtngaygar aarytnatgc cnwsngcncc ngtnaayacn | 1080 |
| | acnacntayg tngcngayac ngarwsngar cargcngaya cntgggayyt nwsngarmgn | 1140 |
| | ccnaargara thaargtnws naaratggar caraarttym gnatgytnws ncargaygcn | 1200 |
| | ccnacngtna argarwsntg yaaracnwsn wsnaayaaya aywsnatggt nwsnaayacn | 1260 |
| | ytngcnaara tgmgnathcc naaytaycar ytnwsnccna cnaarytncc nwsnathaay | 1320 |
| | aarwsnaarg aymgngcnws ncarcarcar caracnaayw snathmgnaa ytayttycar | 1380 |
| | ccnwsnacna araarmgnga rmgngaygar garaaycarg aratgwsnws ntgyaarwsn | 1440 |
| | gcnmgnathg aracnwsntg ywsnytnytn garcaracnc arccngcnac nccnwsnytn | 1500 |
| | tggaaraaya argarcarca yytnwsngar aaygarccng tngayacnaa ywsngayaay | 1560 |
| | aayytnttya cngayacnga yytnaarwsn athgtnaara aywsngcnws naarwsncay | 1620 |
| | gcngcngara arytnmgnws naayaaraar mgngaratgg aygaygtngc nathgargay | 1680 |
| | gargtnytng arcarytntt yaargayacn aarccngary tngarathga ygtnaargtn | 1740 |
| | caraarcarg argargaygt naaygtnmgn aarmgnccnm gnatggayat hgaracnaay | 1800 |
| | gayacnttyw sngaygargc ngtnccngar wsnwsnaara thwsncarga raaygarath | 1860 |
| | ggnaaraarm gngarytnaa rgargaywsn ytntggwsng cnaargarat hwsnaayaay | 1920 |
| | gayaarytnc argaygayws ngaratgytn ccnaaraary tnytnytnac ngarttymgn | 1980 |
| | wsnytngtna thaaraayws nacnwsnmgn aayccnwsng gnathaayga ygaytayggn | 2040 |
| | carytnaara ayttyaaraa rttyaaraar gtnacntayc cnggngcngg naarytnccn | 2100 |
| | cayathathg gnggnwsnga yytnathgcn caycaygcnm gnaaraayac ngarytngar | 2160 |
| | gartggytnm gncargarat ggargtncar aaycarcayg cnaargarga rwsnytngcn | 2220 |
| | gaygayytnt tymgntayaa yccntayytn aarmgnmgnm gn | 2262 |
| SEQ ID NO: 20 | atgtggaary tnytnccngc ngcnggnccn gcnggnggng arccntaymg nytnytnacn | 60 |
| | ggngtngart aygtngtngg nmgnaaraay tgygcnathy tnathgaraa ygaycarwsn | 120 |
| | athwsnmgna aycaygcngt nytnacngcn aayttywsng tnacnaayyt nwsncaracn | 180 |
| | gaygarathc cngtnytnac nytnaargay aaywsnaart ayggnacntt ygtnaaygar | 240 |
| | garaaratgc araayggntt ywsnmgnacn ytnaarwsng gngayggnat hacnttyggn | 300 |
| | gtnttyggnw snaarttymg nathgartay garccnytng tngcntgyws nwsntgyytn | 360 |
| | gaygtnwsng gnaaracngc nytnaaycar gcnathytnc arytnggngg nttyacngtn | 420 |
| | aayaaytgga cngargartg yacncayytn gtnatggtnw sngtnaargt nacnathaar | 480 |
| | acnathtgyg cnytnathtg yggnmgnccn athgtnaarc cngartaytt yacngartty | 540 |
| | ytnaargcng tngarwsnaa raarcarccn ccncarathg arwsnttyta yccnccnytn | 600 |
| | gaygarccnw snathggnws naaraaygtn gayytnwsng gnmgncarga rmgnaarcar | 660 |
| | athttyaarg gnaaracntt yathttyytn aaygcnaarc arcayaaraa rytnwsnwsn | 720 |
| | gcngtngtnt tyggnggngg ngargcnmgn ytnathacng argaraayga rgargarcay | 780 |
| | aayttyttyy tngcnccngg nacntgygtn gtngayacng gnathacnaa ywsncaracn | 840 |
| | ytnathccng aytgycaraa raartggath carwsnatha tggayatgyt ncarmgncar | 900 |
| | ggnytnmgnc cnathccnga rgcngarath ggnytngcng tnathttyat gacnacnaar | 960 |
| | aaytaytgyg ayccncargg ncayccnwsn acnggnytna aracnacnac nccnggnccn | 1020 |
| | wsnytnwsnc arggngtnws ngtngaygar aarytnatgc cnwsngcncc ngtnaayacn | 1080 |
| | acnacntayg tngcngayac ngarwsngar cargcngaya cntgggayyt nwsngarmgn | 1140 |
| | ccnaargara thaargtnws naaratggar caraarttym gnatgytnws ncargaygcn | 1200 |
| | ccnacngtna argarwsntg yaaracnwsn wsnaayaaya aywsnatggt nwsnaayacn | 1260 |
| | ytngcnaara tgmgnathcc naaytaycar ytnwsnccna cnaarytncc nwsnathaay | 1320 |
| | aarwsnaarg aymgngcnws ncarcarcar caracnaayw snathmgnaa ytayttycar | 1380 |
| | ccnwsnacna araarmgnga rmgngaygar garaaycarg aratgwsnws ntgyaarwsn | 1440 |
| | gcnmgnathg aracnwsntg ywsnytnytn garcaracnc arccngcnac nccnwsnytn | 1500 |
| | tggaaraaya argarcarca yytnwsngar aaygarccng tngayacnaa ywsngayaay | 1560 |
| | aayytnttya cngayacnga yytnaarwsn athgtnaara aywsngcnws naarwsncay | 1620 |
| | gcngcngara arytnmgnws naayaaraar mgngaratgg aygaygtngc nathgargay | 1680 |
| | gargtnytng arcarytntt yaargayacn aarccngary tngarathga ygtnaargtn | 1740 |
| | caraarcarg argargaygt naaygtnmgn aarmgnccnm gnatggayat hgaracnaay | 1800 |
| | gayacnttyw sngaygargc ngtnccngar wsnwsnaara thwsncarga raaygarath | 1860 |
| | ggnaaraarm gngarytnaa rgargaywsn ytntggwsng cnaargarat hwsnaayaay | 1920 |
| | gayaarytnc argaygayws ngaratgytn ccnaaraary tnytnytnac ngarttymgn | 1980 |
| | wsnytngtna thaaraayws nacnwsnmgn aayccnwsng gnathaayga ygaytayggn | 2040 |
| | carytnaara ayttyaaraa rttyaaraar gtnacntayc cnggngcngg naarytnccn | 2100 |
| | cayathathg gnggnwsnga yytnathgcn caycaygcnm gnaaraayac ngarytngar | 2160 |
| | gartggytnm gncargarat ggargtncar aaycarcayg cnaargarga rwsnytngcn | 2220 |
| | gaygayytnt tymgntayaa yccntayytn aarmgnmgnm gn | 2262 |

### Methods for the Treatment of Cancers Associated with Reduced MRN Complex Formation and Functionality

The present disclosure provides therapies that involve administering a composition comprising one or more cytotoxic compounds to a human patient for treating a cancer that is associated with reduced MRN complex formation and/or functionality. Cancers that may be treated by the methods disclosed herein include breast cancers, such as hormone-negative breast cancers (HNBCs) and triple-negative breast cancers (TNBCs); and other cancers, such as colorectal cancers and urethecal cancers, which exhibit reduced MRN complex formation and/or functionality.

The term clastogenic therapy refers to well-known cancer therapies which cause cell cycle blockage and/or arrest, and/or apoptosis by damaging DNA. Clastogenic therapies include radiation therapy (e.g., radiotherapy) and certain chemotherapies. Examples of DNA damaging chemotherapy agents that can be used in the methods of the present disclosure include alkylating agents such as nitrogen mustards (e.g., cyclophosphamide), nitrosoureas, alkyl sulfonates, triazines, ethylenimines, and platinum coordination complexes; anti-metabolites such as pyrimidine and purine compounds as well as folate antagonists. Additional chemotherapy agents that do not cause DNA damage can also be employed such as those that block the cell cycle or otherwise prevent mitosis and/or promote apoptosis (e.g., kinase inhibitors and vinca alkaloids), and anti-metabolites such as methotrexate and 5-FU. In one embodiment the chemotherapy agent comprises cyclophosphamide and an anti-metabolite such as methotrexate and 5-FU. In another embodiment the chemotherapy agent comprises a platinum coordination complex, such as cisplatin or carboplatin. In a further embodiment the chemotherapy agent comprises epirubicin, cyclophosphamide, and 5-FU.

The clastogenic therapy (e.g., chemotherapy agent and/or radiation therapy) can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

The amount of a cytotoxic compound that will be effective in the treatment, inhibition, and/or prevention of a cancer associated with reduced MRN complex formation and/or functionality can be determined by standard clinical techniques. In vitro assays may optionally be employed to help identify optimal dosage ranges. The precise dose to be employed in the formulation will also depend on the route of administration, and the seriousness of the disease or disorder. Effective doses may be extrapolated from dose-response curves derived from in vitro or animal model test systems.

The compounds or compositions of the present disclosure can be tested in vitro, and then in vivo for the desired therapeutic or prophylactic activity, prior to use in humans. For example, in vitro assays to demonstrate the therapeutic or prophylactic utility of a cytotoxic compound or composition include the effect of a cytotoxic compound on a cell line or a patient tissue sample. The effect of the cytotoxic compound or composition on the cell line and/or tissue sample can be determined utilizing techniques known to those of skill in the art including, but not limited to growth and survival assays. In accordance with the present disclosure, in vitro assays that can be used to determine whether administration of a specific cytotoxic compound is indicated, include in vitro cell culture assays in which a patient tissue sample is grown in culture, and exposed to or otherwise administered a cytotoxic compound, and the effect of such cytotoxic compound upon the tissue sample is observed.

The present disclosure provides methods of treatment and inhibition by administration to a patient of an effective amount of a cytotoxic compound or composition as described herein. In one aspect, the cytotoxic compound is substantially purified such that the compound is substantially free from substances that limit its effect or produce undesired side-effects.

Various delivery systems are known and can be used to administer a composition of the present disclosure, for example, encapsulation in liposomes, microparticles, microcapsules, receptor-mediated endocytosis (see, e.g., Wu and Wu, J Biol. Chem. 262:4429-4432 (1987)), and the like as will be known by one of skill in the art.

Methods of administration include, but are not limited to, intradermal, intramuscular, intraperitoneal, intravenous, subcutaneous, intranasal, epidural, and oral routes. The cytotoxic compounds or compositions may be administered by any convenient route, for example by infusion or bolus injection, by absorption through epithelial or mucocutaneous linings (e.g., oral mucosa, rectal and intestinal mucosa, etc.) and may be administered together with other therapeutically effective compounds, such as Adriamycin and/or Taxol. Administration can be systemic or local. In addition, it may be desirable to introduce the clastogenic compounds or compositions into the central nervous system by any suitable route, including intraventricular and intrathecal injection. Intraventricular injection may be facilitated by an intraventricular catheter, for example, attached to a reservoir, such as an Ommaya reservoir. Pulmonary administration can also be employed, for example, by use of an inhaler or nebulizer, and formulation with an aerosolizing agent.

It may be desirable to administer the clastogenic compounds or compositions of locally to the area in need of treatment; this may be achieved by, for example, local infusion during surgery, topical application, by injection, by means of a catheter, by means of a suppository, or by means of an implant, said implant being of a porous, non-porous, or gelatinous material, including membranes, such as sialastic membranes, or fibers.

The clastogenic compound can be delivered in a vesicle, such as a liposome (Langer, Science 249:1527-1533 (1990)) or in a controlled release system. A controlled release system can be placed in proximity of the therapeutic target, thus requiring only a fraction of the systemic dose (see, e.g., Goodson, in Medical Applications of Controlled Release, Vol. 2, pp. 115-138 (1984)).

Intravenous infusion of a compositions comprising a clastogenic compound may be continuous for a duration of at least about one day, or at least about three days, or at least about seven days, or at least about 14 days, or at least about 21 days, or at least about 28 days, or at least about 42 days, or at least about 56 days, or at least about 84 days, or at least about 112 days.

Continuous intravenous infusion of a composition comprising a clastogenic compound may be for a specified duration, followed by a rest period of another duration. For example, a continuous infusion duration may be from about 1 day, to about 7 days, to about 14 days, to about 21 days, to about 28 days, to about 42 days, to about 56 days, to about 84 days, or to about 112 days. The continuous infusion may then be followed by a rest period of from about 1 day, to about 2 days to about 3 days, to about 7 days, to about 14 days, or to about 28 days. Continuous infusion may then be repeated, as above, and followed by another rest period.

Regardless of the precise continuous infusion protocol adopted, it will be understood that continuous infusion of a composition comprising a clastogenic compound will continue until either desired efficacy is achieved or an unacceptable level of toxicity becomes evident.

In another aspect of the present disclosure where it has been determined that the cancer has enhanced susceptibility to DNA damaging chemotherapy agents, the cancer may also be identified as being susceptible to reduced (less intense) chemotherapy regimen. The term reduced chemotherapy regimen is herein understood to mean a reduced dosage of chemotherapy agents and/or a reduced schedule of administration of the chemotherapy agents as compared to a normal regimen that would be selected for a given patient by a physician. Reducing the dosage and/or schedule of a chemotherapy regimen given to a patient will reduce the harmful side effects of the drugs, thereby improving their quality of life during and post treatment. It will be appreciated that where the cancer is identified as being treated by a reduced chemotherapy regimen, that the ultimate determination of the treatment protocol for an individual patient will incorporate a medical provider's understanding of other factors including, among others, family history, patient age, and overall health and fitness.

It will be understood that, unless indicated to the contrary, terms intended to be “open” (e.g., the term “including” should be interpreted as “including but not limited to,” the term “having” should be interpreted as “having at least,” the term “includes” should be interpreted as “includes but is not limited to,” etc.). Phrases such as “at least one,” and “one or more,” and terms such as “a” or “an” include both the singular and the plural.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

All references cited herein, whether supra or infra, including, but not limited to, patents, patent applications, and patent publications, whether U.S., PCT, or non-U.S. foreign, and all technical and/or scientific publications are hereby incorporated by reference in their entirety.

While various embodiments have been disclosed herein, other embodiments will be apparent to those skilled in the art. The various embodiments disclosed herein are for purposes of illustration and are not intended to be limiting, with the true scope and spirit being indicated by the claims.

The present disclosure will be further described with reference to the following non-limiting examples. The teaching of all patents, patent applications and all other publications cited herein are incorporated by reference in their entirety.

### EXAMPLES

### Example 1

### Triple-Negative Breast Cancers Exhibiting Reduced MRN Complex Levels

This Example demonstrates that hormone negative breast cancers (e.g., triple negative breast cancers) can contain a somatic mutation in the gene(s) encoding the MRN complex and/or its component proteins which lead to a detectable decrease in MRN complex expression levels in the tumor.

There is growing appreciation that triple negative (ER−/PR−/Her2 non-amplified) breast cancer is a heterogeneous entity, particularly in regards to the response to DNA damaging chemotherapeutic agents. Differences in response rates to neoadjuvant chemotherapy are clinically significant, as patients who achieve a complete pathological response have a markedly improved disease-free and overall survival compared to those patients who do not achieve a complete response.

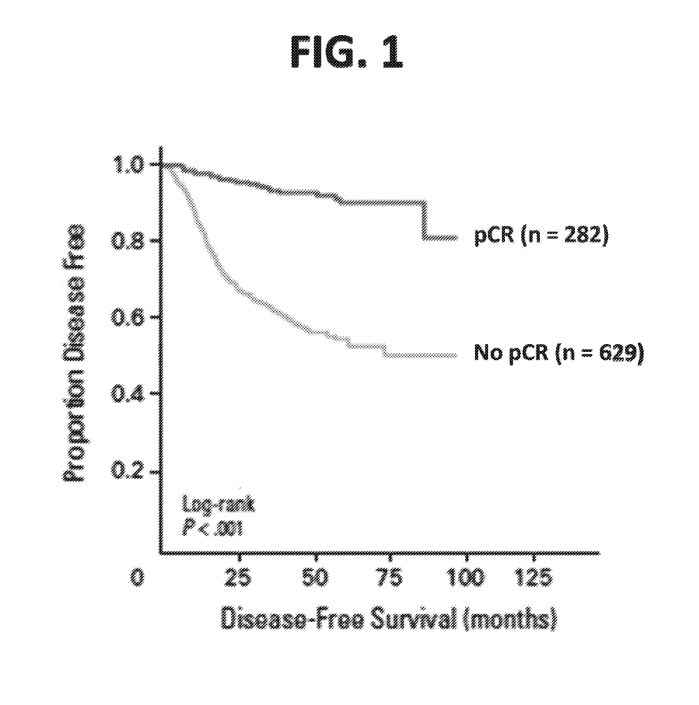

FIG. 1 is a graph showing disease-free survival of triple negative breast cancer (TNBC) patients treated with neoadjuvant chemotherapy who either achieve a pathological complete response (pCR) or do not achieve a pathological complete response (no pCR). Taken from von Minckwitz et al., JCO 30:1796-1804 (2013).

Prior to the discoveries leading to the present disclosure, there were no molecular markers that were able to predict chemotherapeutic response in triple negative breast cancer (TNBC). Such markers would be particularly useful in the majority of TNBC patients who receive chemotherapy after surgery, for whom there was no effective predictor of response to chemotherapy. Recent work has attempted to sub-classify TNBC into distinct genetic subtypes, but emerging data indicates that these genetic differences do not completely explain the heterogeneity in chemotherapy response (Lehmann et al., JCI 121:2750-67 (2011), and Masuda et al., CCR 19:5533-40 (2013)).

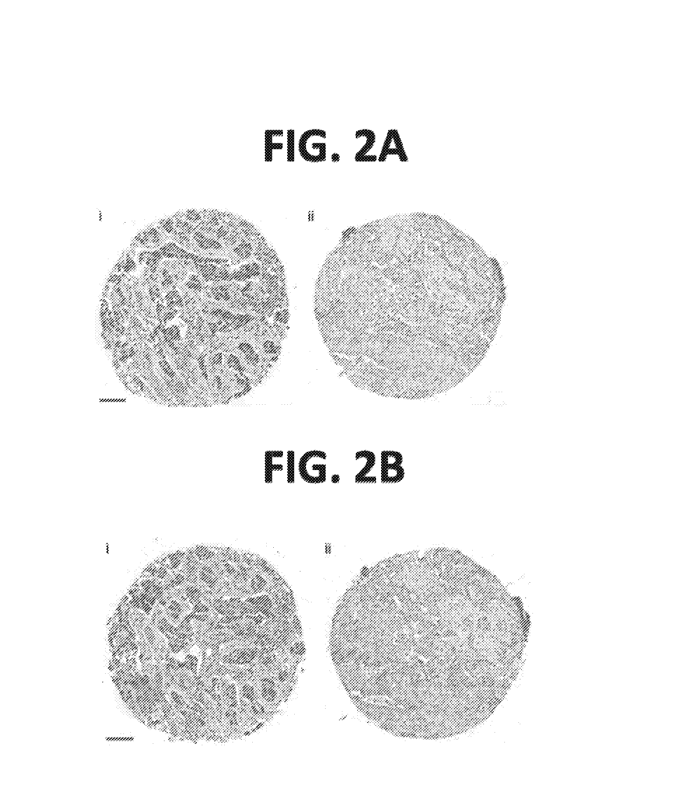

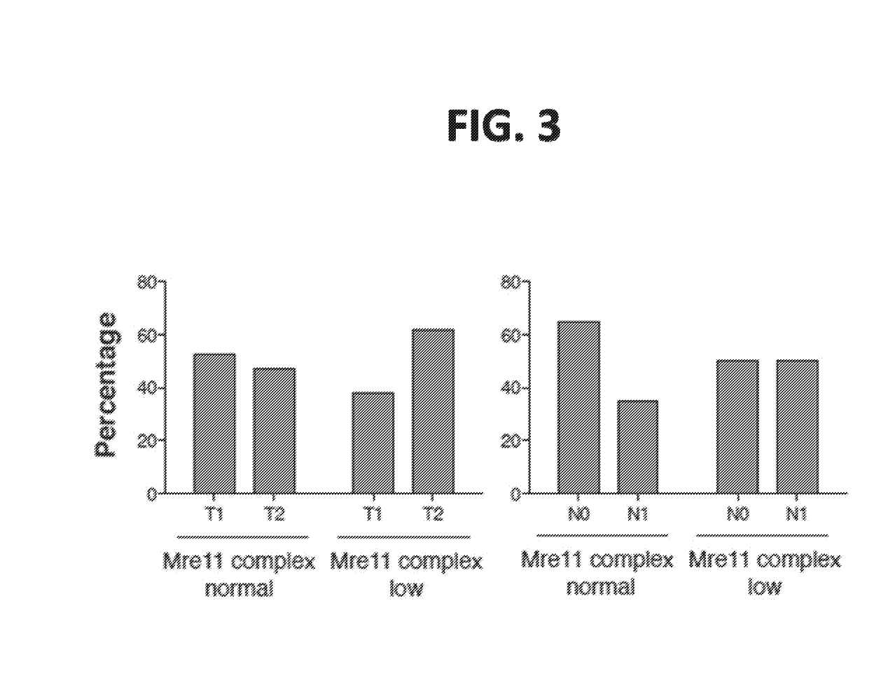

As described herein, it was discovered that the MRN complex represents an informative predictor of chemotherapy response in this heterogeneous breast cancer subtype. MRE11 immunohistochemistry (IHC) analyses of triple-negative breast cancer (TNBC) tissue microarrays with a representative breast tumor having a normal level of the MRE11 complex or an abnormally low level of the MRE11 complex demonstrated that low MRE11 staining (FIG. 2A) and low NBS1 staining (FIG. 2B) correlate with improved overall survival in TNBC. The distribution of tumor stage and nodal stage in breast tumors expressing normal levels of the MRE11 complex vs. low levels of the MRE11 complex is shown in FIG. 3.

The clinical utility of using MRN as a predictor of chemotherapy response in TNBC include: (a) prognostic information when chemotherapy is given in the adjuvant setting; (b) chemotherapy regimen modification to reduce toxicity and increase tumor response based on the particular sensitivity of Mre11-deficient breast cancers; and (c) enrollment on clinical trials for patients who are Mre11+, as these patients are expected to have worse outcomes than Mre11^{low} patients.

While chemotherapy has proven to be therapeutically beneficial in treatment of hormone negative breast cancers in general, treatment of patients with hormone negative breast tumors which have reduced MRN complex expression is particularly successful (e.g., they exhibit superior survival rates after these therapies).

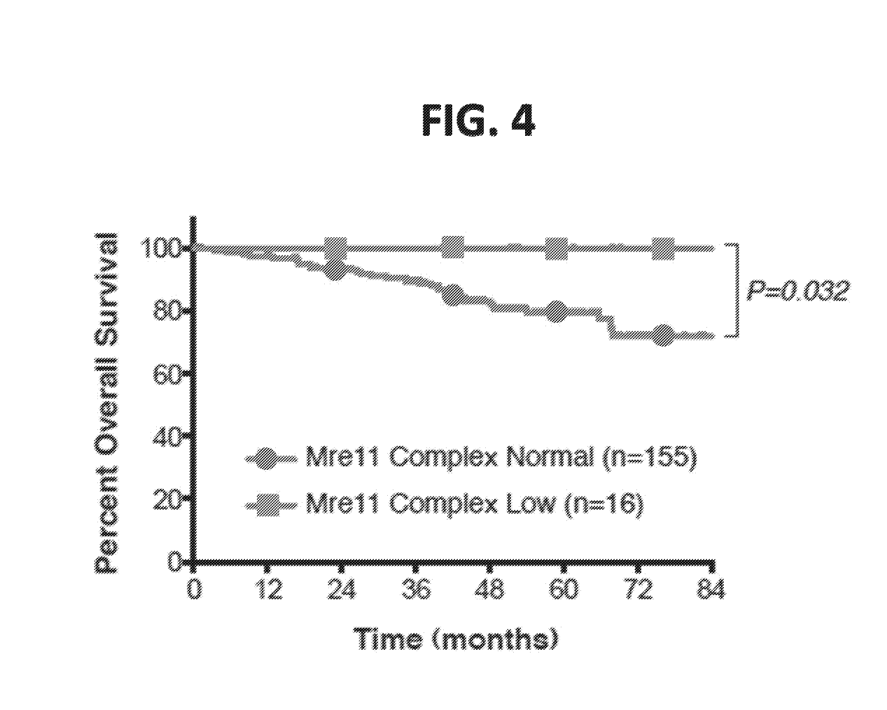

A tissue microarray (TMA) of 155 hormone negative breast cancer tumors (e.g., triple negative breast cancer (TNBC) tumors) from patients treated at Memorial Sloan-Kettering Cancer Center (MSKCC), with annotated clinical follow-up was assembled. Immunohistochemical analysis of the MRN complex protein levels, MRE11 and NBS1, in the TNBC TMA identified a subset of tumors (16/155; 10.3%) with unexpectedly low expression of both. RAD50, the third protein member of the MRN complex was not examined, but the coincident reduction in MRE11 and NBS1 levels suggests overall destabilization of the MRN complex in the affected tumors and likewise a corresponding decrease in the content of the component proteins. FIG. 4.

MRN complex reduction correlated strongly with the enhanced susceptibility of the cancer to clastogenic therapy. The majority of patients represented on the TMA received adjuvant chemotherapy and/or radiation therapy as part of their breast cancer therapy (MRN complex low: 15/16, 94%; MRN complex normal: 156/175, 89%). Patients with the MRN complex-low tumors exhibited improved overall survival and distant metastasis-free survival relative to patients with the MRN normal tumors.

These data suggest that epigenetic changes and/or somatic mutations (e.g., non-germline mutations) in the gene(s) encoding the MRN complex and/or its components proteins leads to MRN complex hypomorphism which can promote tumorigenesis. It also suggests that the same epigenetic changes and/or somatic mutations renders the resulting tumors more susceptible to therapies with one or more cytotoxic agent.

### Example 2

### Mre11 Immunohistochemistry as a Predictor of Chemotherapy Response in ER⁻/PR⁻/HER2⁻ Breast Cancer

This Example demonstrates that cells having epigenetic changes and/or somatic mutations causing reduced expression of the MRN complex, and/or its component proteins, exhibit enhanced susceptibility to some, but not all, clastogenic or other cytoxic agents.

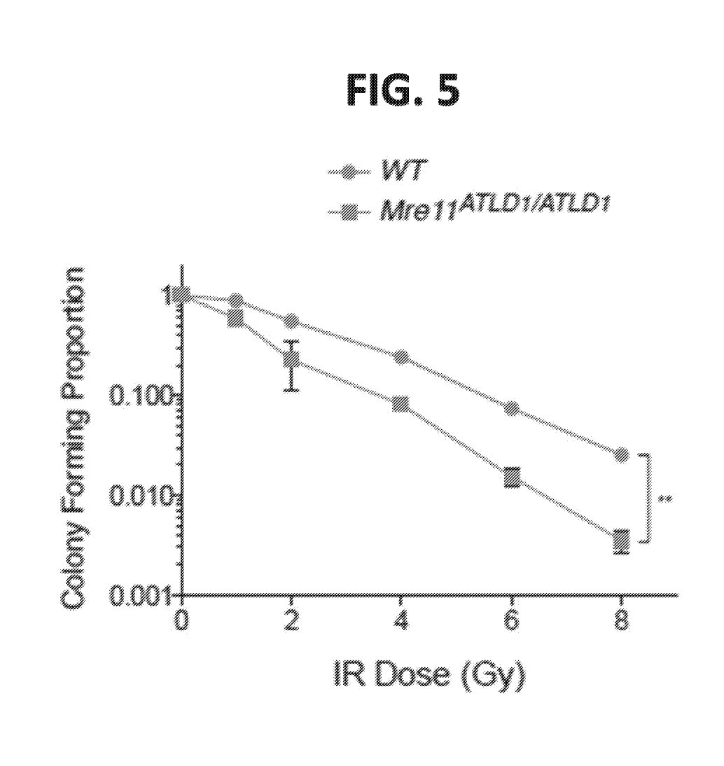

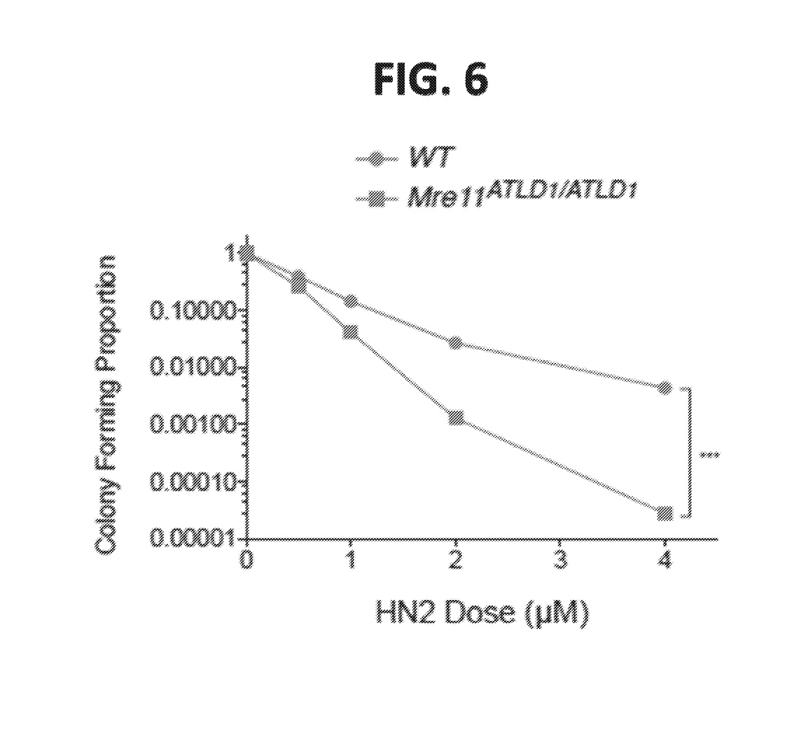

Murine embryonic fibroblast cells that express a low level of Mre11 (Mre11-impaired; Mre11^{ATLD1/ATLD1}) exhibited enhanced susceptibility to IR exposure and to the DNA damaging agent Mechlorethamine (H2N) as compared to murine embryonic fibroblast cells that express a normal level of Mre11 (WT). FIG. 5 and FIG. 6, respectively.

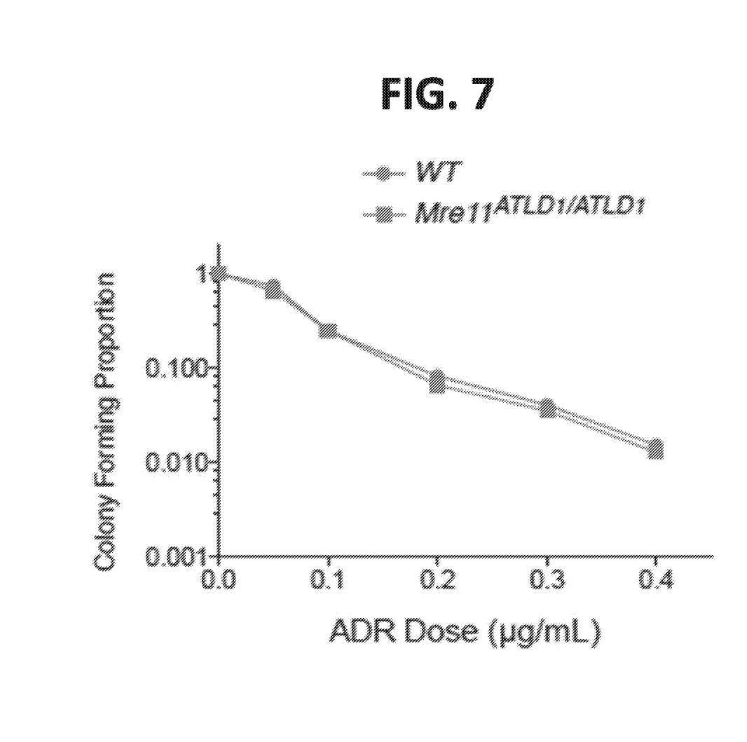

In contrast, murine embryonic fibroblast cells that express a low level of Mre11 (Mre11-impaired; Mre11^{ATLD1/ATLD1}) exhibit an equivalent response (i.e., no difference in susceptibility) to Adriamycin (ADR) as compared to murine embryonic fibroblast cells that express a normal level of Mre11 (WT). FIG. 7.

What is claimed is:

1. A method for identifying a cancer cell that is susceptible to growth or survival inhibition by a cytotoxic agent, said method comprising:
a. detecting a level of MRN complex formation in a cancer cell,
b. detecting a level of MRN complex formation in a non-cancer cell, and
c. comparing said level of MRN complex formation in said cancer cell and said level of MRN complex formation in said non-cancer cell;
wherein a reduced MRN complex formation in said cancer cell as compared to said non-cancer cell indicates that said cancer cell is susceptible to growth or survival inhibition by said cytotoxic agent.

2. The method of claim 1 wherein said detecting of MRN complex formation comprises contacting said cancer cell and said non-cancer cell with an antibody that binds to a human MRE11 protein, a human RAD50 protein, or a human NBS1 protein.

3. The method of claim 2 wherein said antibody comprises a fluorescent label.

4. The method of claim 3 wherein said detecting further comprises detecting said fluorescent label wherein one or more fluorescent foci within in a nucleus of said cell indicates the formation of an MRN complex formation in said cell.

5. The method of claim 4 wherein a decreased number of foci in said cancer cell as compared to said normal cell indicates the susceptibility of said cancer cell to growth or survival inhibition by said cytotoxic agent.

6. A method for identifying a cancer cell that is susceptible to growth or survival inhibition by a cytotoxic agent, said method comprising:
a. detecting a level of MRN complex formation and/or functionality in said cancer cell,
b. detecting a level of MRN complex formation and/or functionality in a non-cancer cell, and
c. comparing the level of MRN complex formation and/or functionality in said cancer cell and the level of MRN complex formation and/or functionality in said non-cancer cell;
wherein a reduced level of MRN complex formation and/or functionality in said cancer cell as compared to said non-cancer cell indicates that said cancer cell is susceptible to growth or survival inhibition by said cytotoxic agent.

7. The method of claim 6, said detecting comprising determining the level of expression of a gene selected from the group consisting of an Mre11 gene, a Rad50 gene, and an Nbs1 gene in said cancer cell and in said non-cancer cell.

8. The method of claim 7 wherein said level of gene expression is determined by a step of hybridizing a primer to a nucleotide sequence encoded by said Mre11, Rad50, and/or Nbs1 gene.

9. The method of claim 8 wherein said Mre11 gene encodes an MRE11 protein comprising an amino sequence selected from the group consisting of SEQ ID NO: 1, SEQ ID NO: 2, SEQ ID NO: 3, and SEQ ID NO: 4.

10. The method of claim 8 wherein said Rad50 gene encodes a RAD50 protein comprising an amino sequence selected from the group consisting of SEQ ID NO: 5, SEQ ID NO: 6, and SEQ ID NO: 7.

11. The method of claim 8 wherein said Nbs1 gene encodes an NBS1 protein comprising an amino sequence selected from the group consisting of SEQ ID NO: 8, SEQ ID NO: 9, and SEQ ID NO: 10.

12. The method of claim 6 wherein said reduced MRN complex formation or functionality results from a reduced cellular level of a protein selected from the group consisting of an MRE11 protein, a RAD50 protein, and an NBS1 protein. in said cancer cell as compared to said protein in a non-cancer cell.

13. The method of claim 12 wherein said cellular level of said protein is determined by a step if binding an antibody to said protein.

14. The method of claim 13 wherein said antibody binds to an MRE11 protein comprising an amino sequence selected from the group consisting of SEQ ID NO: 1, SEQ ID NO: 2, SEQ ID NO: 3, and SEQ ID NO: 4.

15. The method of claim 13 wherein said antibody binds to a RAD50 protein comprising an amino sequence selected from the group consisting of SEQ ID NO: 5, SEQ ID NO: 6, and SEQ ID NO: 7.

16. The method of claim 13 wherein said antibody binds to an NBS1 protein comprising an amino sequence selected from the group consisting of SEQ ID NO: 8, SEQ ID NO: 9, and SEQ ID NO: 10.

17. The method of claim 6 wherein said reduced MRN complex formation and/or functionality results from a mutation, an insertion, and/or a deletion in a gene in said cancer cell as compared to said gene in said non-cancer cell, wherein said gene is selected from the group consisting of an Mre11 gene, a Rad50 gene, and an Nbs1 gene.

18. The method of claim 17 wherein said mutation, insertion, and/or deletion in said gene reduces or eliminates a function of a protein selected from the group consisting of an MRE11, RAD50, and NBS1 in said cancer cell as compared to said protein in said non-cancer cell.

19. A method for identifying in a patient having a cancer the susceptibility of said cancer to a cytotoxic agent, said method comprising:
a. detecting a level of MRN complex functionality in a cell from said cancer,
b. detecting a level of MRN complex functionality in a non-cancer cell, and
c. comparing the level of MRN complex functionality in said cancer cell and the level of MRN complex functionality in said non-cancer cell;
wherein a reduced level of MRN complex functionality in said cancer cell as compared to said non-cancer cell indicates that said cancer cell is susceptible to growth or survival inhibition by said cytotoxic agent.

20. A method for inhibiting the growth and/or survival of a cancer cell that exhibits reduced MRN complex formation and/or functionality, said method comprising: contacting said cancer cell with a cytotoxic agent, wherein said reduced MRN complex formation and/or functionality renders said cancer cell susceptible to growth and/or survival inhibition by said cytotoxic agent.

21. The method of claim 20 wherein said cytotoxic agent is a clastogenic agent.

22. The method of claim 21 wherein said clastogenic agent is a clastogenic compound or a source of ionizing radiation.

23. The method of claim 22 wherein said clastogenic compound is selected from the group consisting of an alkylating agent, a topoisomerase I inhibitor, and a crosslinking agent.

24. The method of claim 23 wherein said alkylating agent is selected from the group consisting of cyclophosphamide (CP), mechlorethamine, chlorambucil, methyl methanesulfonate (MMS), and melphalan.

25. The method of claim 23 wherein said topoisomerase I inhibitor is selected from the group consisting of irinotecan, topotecan, camptothecin, and lamellarin D.

26. The method of claim 23 wherein said crosslinking agent is selected from the group consisting of carboplatin, cisplatin, oxaliplatin, and mitomycin C (MMC).

27. The method of claim 20, further comprising contacting said cell with a second cytotoxic agent.

28. The method of claim 27 wherein said second cytotoxic agent is a second clastogenic agent selected from the group consisting of an alkylating agent, a topoisomerase I inhibitor, and a cross-linking agent.

29. The method of claim 27 wherein said second cytotoxic agent is selected from the group consisting of a PARP inhibitor, an ATM inhibitor, an ATR inhibitor, a DNA-PK inhibitor, a Chk1 inhibitor, and a homologous recombination inhibitor.

30. A method for treating a cancer patient, said method comprising: administering to said cancer patient a cytotoxic agent or a composition comprising a cytotoxic agent, wherein said cancer exhibits reduced MRN complex formation and/or functionality as compared to a non-cancer.

31. The method of claim 30 wherein said cytotoxic agent is a clastogenic agent.

32. The method of claim 31 wherein said clastogenic agent is a clastogenic compound or a source of ionizing radiation.

33. The method of claim 32 wherein said clastogenic compound is selected from the group consisting of an alkylating agent, a topoisomerase I inhibitor, and a crosslinking agent.

34. The method of claim 33 wherein said alkylating agent is selected from the group consisting of cyclophosphamide (CP), mechlorethamine, chlorambucil, methyl methanesulfonate (MMS), and melphalan.

35. The method of claim 34 wherein said topoisomerase I inhibitor is selected from the group consisting of irinotecan, topotecan, camptothecin, and lamellarin D.

36. The method of claim 34 wherein said crosslinking agent is selected from the group consisting of carboplatin, cisplatin, oxaliplatin, and mitomycin C (MMC).

37. The method of claim 30, further comprising contacting said cell with a second cytotoxic agent.

38. The method of claim 37 wherein said second cytotoxic agent is a second clastogenic agent selected from the group consisting of an alkylating agent, a topoisomerase I inhibitor, and a cross-linking agent.

39. The method of claim 37 wherein said second cytotoxic agent is selected from the group consisting of a PARP inhibitor, an ATM inhibitor, an ATR inhibitor, a DNA-PK inhibitor, a Chk1 inhibitor, and a homologous recombination inhibitor.

40. A method for treating a cancer patient, said method comprising: administering to said cancer patient a cytotoxic agent or a composition comprising a cytotoxic agent, wherein said cancer exhibits reduced MRN complex formation and/or functionality as compared to a non-cancer.

41. The method of claim 40 wherein said cytotoxic agent is a clastogenic agent.

42. The method of claim 41 wherein said clastogenic agent is a clastogenic compound or a source of ionizing radiation.

43. The method of claim 42 wherein said clastogenic compound is selected from the group consisting of an alkylating agent, a topoisomerase I inhibitor, and a crosslinking agent.

44. The method of claim 43 wherein said alkylating agent is selected from the group consisting of cyclophosphamide (CP), mechlorethamine, chlorambucil, methyl methanesulfonate (MMS), and melphalan.

45. The method of claim 43 wherein said topoisomerase I inhibitor is selected from the group consisting of irinotecan, topotecan, camptothecin, and lamellarin D.

46. The method of claim 43 wherein said crosslinking agent is selected from the group consisting of carboplatin, cisplatin, oxaliplatin, and mitomycin C (MMC).

47. The method of claim 40, further comprising contacting said cell with a second cytotoxic agent.

48. The method of claim 47 wherein said second cytotoxic agent is a second clastogenic agent selected from the group consisting of an alkylating agent, a topoisomerase I inhibitor, and a cross-linking agent.

49. The method of claim 47 wherein said second cytotoxic agent is selected from the group consisting of a PARP inhibitor, an ATM inhibitor, an ATR inhibitor, a DNA-PK inhibitor, a Chk1 inhibitor, and a homologous recombination inhibitor.

50. A method for identifying a cytotoxic compound to which a cancer cell exhibiting reduced MRN complex formation and/or functionality has enhanced sensitivity compared to a cancer cell of the same type not exhibiting reduced MRN complex formation and/or functionality, said method comprising contacting said cancer cell with said cytotoxic compound and assessing one or more of colony formation, level of 53BP1 foci formed, induction of chromosome aberrations, and micronucleus formation.

51. A composition, comprising:
(a) a first clastogenic cancer therapeutic compound,
(b) a second clastogenic cancer therapeutic compound, and
(c) a first non-clastogenic cancer therapeutic compound.

52. The composition of claim 51 wherein said first clastogenic compound and said second clastogenic compound are independently selected from the group consisting of an alkylating agent, a topoisomerase I inhibitor, and a DNA cross-linking agent.

53. The composition of claim 51 wherein said first clastogenic compound is an alkylating agent and wherein said second clastogenic agent is a topoisomerase I inhibitor.

54. The composition of claim 51 wherein said first clastogenic compound is an alkylating agent and wherein said second clastogenic agent is a cross-linking agent.

55. The composition of claim 51 wherein said first clastogenic compound is an topoisomerase I inhibitor and wherein said second clastogenic agent is a cross-linking agent.

56. The composition of claim 51 wherein said first non-clastogenic compound is selected from the group consisting of a PARP inhibitor, an ATM inhibitor, an ATR inhibitor, a DNA-PK inhibitor, a Chk1 inhibitor, and a homologous recombination inhibitor.

57. The composition of claim 51 wherein said first clastogenic compound is cyclophosphamide (CP).

58. The composition of claim 57 wherein said second clastogenic compound is mechlorethamine, chlorambucil, methyl methanesulfonate (MMS), and melphalan.

59. The composition of claim 51 wherein said topoisomerase I inhibitor is selected from the group consisting of irinotecan, topotecan, camptothecin, and lamellarin D.

60. The composition of claim 51 wherein said crosslinking agent is selected from the group consisting of carboplatin, cisplatin, oxaliplatin, and mitomycin C (MMC).

61. A composition, comprising:
(a) a first clastogenic compound,
(b) a first non-clastogenic compound, and
(c) a second non-clastogenic compound.

62. The composition of claim 61 wherein said first clastogenic compound is selected from the group consisting of an alkylating agent, a topoisomerase I inhibitor, and a cross-linking agent.

63. The composition of claim 62 wherein said first clastogenic compound is an alkylating agent selected from the group consisting of cyclophosphamide (CP), mechlorethamine, chlorambucil, methyl methanesulfonate (MMS), and melphalan.

64. The composition of claim 61 wherein said first and said second non-clastogenic compounds are each nucleotide analogs independently selected from the group consisting of azacitidine, azathioprine, capecitabine, cytarabine, doxifluridine, fluorouracil, gemcitabine, hydroxyurea, mercaptopurine, methotrexate, and thioguanine.

65. The composition of claim 61 wherein said first clastogenic compound is cyclophosphamide (CP), wherein said first non-clastogenic compound is methotrexate, and wherein said second non-clastogenic compound is fluorouracil.

66. The composition of claim 61 wherein said first clastogenic compound is cyclophosphamide (CP), wherein said first non-clastogenic compound is an anthracycline, and wherein said second non-clastogenic compound is a nucleotide analog.

67. The composition of claim 66 wherein said anthracycline is selected from the group consisting of daunorubicin, doxorubicin, epirubicin, idarubicin, mitoxantrone, and valrubicin and wherein said nucleotide analog is selected from the group consisting of azacitidine, azathioprine, capecitabine, cytarabine, doxifluridine, fluorouracil, gemcitabine, hydroxyurea, mercaptopurine, methotrexate, and thioguanine.

68. The composition of claim 67 wherein said anthracycline is epirubicin and wherein said nucleotide analog is fluorouracil.

69. A composition, comprising: one or more cytotoxic agents selected from the group consisting of an alkylating agent, a topoisomerase I inhibitor, a cross-linking agent, a nucleotide and precursor analogs, and a DNA damage response (DDR) signaling and repair inhibitor, wherein each of said cytotoxic agents enhances growth and/or survival inhibition in a cell having a reduced level of MRN complex formation and/or functionality as compared to a cell having a normal or wild-type level of MRN complex formation and/or functionality.

70. The composition of claim 69 wherein one or more of said cytotoxic agents is an alkylating agent selected from the group consisting of cyclophosphamide, mechlorethamine, chlorambucil, methyl methanesulfonate (MMS), and melphalan

71. The composition of claim 69 wherein one or more of said cytotoxic agents is a topoisomerase I inhibitor selected from the group consisting of irnotecan, topotecan, camptothecin, and lamellarin D.

72. The composition of claim 69 wherein one or more of said cytotoxic agents is a cross-linking agent selected from the group consisting of cisplatin, carboplatin, oxalplatin, and mitomycin C.

73. The composition of claim 69 wherein one or more of said cytotoxic agents is a nucleotide or precursor analog selected from the group consisting of azacitidine, azathioprine, capecitabine, cytarabine, doxifluriding, fluorouracil (5-FU), gemcitabine, hydroxyurea, mercaptopurine, methotrexate, and thioguanine.

74. The composition of claim 69 wherein one or more of said cytotoxic agents is a DNA damage response (DDR) signaling or repair inhibitor selected from the group consisting of a PARP inhibitor, an ATM inhibitor, an ATR inhibitor, a DNA-PK inhibitor, a Chk1 inhibitor, and a homologous recombination inhibitor.

75. The composition of claim 69 wherein said composition does not comprise any one or more of the cytotoxic agents selected from the group consisting anthracyclines, cytoskeletal disrupters, epothilones, and vinca alkaloids and derivatives, wherein said one or more cytotoxic agents do not provide substantially advantageous growth and/or survival inhibition in a cell having a reduced level of MRN complex formation and/or functionality as compared to a cell having a normal or wild-type level of MRN complex formation and/or functionality.

76. A method for the treatment of a cancer in a patient, said method comprising administering to said patient a composition comprising one or more cytotoxic agents selected from the group consisting of an alkylating agent, a topoisomerase I inhibitor, a cross-linking agent, a nucleotide and precursor analogs, and a DNA damage response (DDR) signaling and repair inhibitor, wherein said cancer comprises a cell exhibiting reduced MRN complex formation and/or functionality as compared to a cell having a normal or wild-type level of MRN complex formation and/or functionality and wherein one or more of said cytotoxic agents exhibits enhanced growth and/or survival inhibition in said cancer cell as compared to a cell exhibiting normal or wild-type MRN complex formation and/or functionality.

77. The method of claim 76 wherein one or more of said cytotoxic agents is an alkylating agent selected from the group consisting of cyclophosphamide, mechlorethamine, chlorambucil, methyl methanesulfonate (MMS), and melphalan.

78. The method of claim 76 wherein one or more of said cytotoxic agents is a topoisomerase I inhibitor selected from the group consisting of irnotecan, topotecan, camptothecin, and lamellarin D.

79. The method of claim 76 wherein one or more of said cytotoxic agents is a cross-linking agent selected from the group consisting of cisplatin, carboplatin, oxalplatin, and mitomycin C.

80. The method of claim 76 wherein one or more of said cytotoxic agents is a nucleotide or precursor analog selected from the group consisting of azacitidine, azathioprine, capecitabine, cytarabine, doxifluriding, fluorouracil (5-FU), gemcitabine, hydroxyurea, mercaptopurine, methotrexate, and thioguanine.

81. The method of claim 76 wherein one or more of said cytotoxic agents is a DNA damage response (DDR) signaling or repair inhibitor selected from the group consisting of a PARP inhibitor, an ATM inhibitor, an ATR inhibitor, a DNA-PK inhibitor, a Chk1 inhibitor, and a homologous recombination inhibitor.

82. The method of claim 76 wherein said composition does not comprise any one or more of the cytotoxic agents selected from the group consisting anthracyclines, cytoskeletal disrupters, epothilones, and vinca alkaloids and derivatives, wherein said one or more cytotoxic agents do not provide substantially advantageous growth and/or survival inhibition in a cell having a reduced level of MRN complex formation and/or functionality as compared to a cell having a normal or wild-type level of MRN complex formation and/or functionality.
